(19) United States
(12) Patent Application Publication
(10) Pub. No.: US 2023/0050179 A1
(43) Pub. Date: Feb. 16, 2023

(54) BIOLOGICAL MEASUREMENT DEVICE, PULSE WAVE SENSOR, SPHYGMOMANOMETER, AND MEETING SUPPORT SYSTEM

(71) Applicants: Toshihiro ISHII, Miyagi (JP); Ikuo KATOH, Kanagawa (JP); Mitsuru SUZUKI, Miyagi (JP)

(72) Inventors: Toshihiro ISHII, Miyagi (JP); Ikuo KATOH, Kanagawa (JP); Mitsuru SUZUKI, Miyagi (JP)

(73) Assignee: Ricoh Company, Ltd., Tokyo (JP)

(21) Appl. No.: 17/816,069

(22) Filed: Jul. 29, 2022

(30) Foreign Application Priority Data
Jul. 30, 2021 (JP) 2021-126082
Jun. 1, 2022 (JP) 2022-089891

(51) Int. Cl. A61B 5/00; A61B 5/021

(52) U.S. Cl. CPC A61B 5/6824; A61B 5/02125; A61B 5/02416

(57) ABSTRACT

A biological-measurement device includes a light-emitting unit configured to emit light on a body of a test-subject, a light-detecting unit configured to detect light reflected in the body of the test-subject, a control-unit configured to calculate information regarding a pulse-wave of the body of the test-subject based on the light detected by the light-detecting unit, a circuit-board that is flexible and has a first-surface on which the light-emitting unit and the light-detecting unit are provided, the circuit-board further having wiring connecting the light-emitting unit and the control-unit together and connecting the light-detecting unit and the control-unit together, a shielding-unit that is provided on the first-surface, the shielding-unit being situated between the light-emitting unit and the light-detecting unit and configured to protrude beyond the light-emitting unit and the light-detecting unit in a direction perpendicular to the first-surface, and an adhesive-part for firmly contacting with the body of the test-subject.

### CROSS-REFERENCE TO RELATED APPLICATION

The present application is based on and claims priority under 35 U.S.C. § 119 to Japanese Patent Application No. 2021-126082, filed on Jul. 30, 2021, and Japanese Patent Application No. 2022-089891, filed on Jun. 1, 2022. The contents of Japanese Patent Application No. 2021-126082 and Japanese Patent Application No. 2022-089891 are incorporated herein by reference in their entirety.

### BACKGROUND OF THE INVENTION

### 1. Field of the Invention

Embodiments of the present disclosure relate to a biological measurement device, a pulse wave sensor, a sphygmomanometer, and a meeting support system.

### SUMMARY OF THE INVENTION

In one embodiment of the present disclosure, there is provided a biological measurement device that includes:

a light emitting unit configured to emit light on a body of a test subject;

a light detecting unit configured to detect light reflected in the body of the test subject;

a control unit configured to calculate information regarding a pulse wave of the body of the test subject based on the light detected by the light detecting unit;

a circuit board that is flexible and has a first surface on which the light emitting unit and the light detecting unit are provided, the circuit board further having wiring connecting the light emitting unit and the control unit together and connecting the light detecting unit and the control unit together;

a shielding unit that is provided on the first surface, the shielding unit being situated between the light emitting unit and the light detecting unit and configured to protrude beyond the light emitting unit and the light detecting unit in a direction perpendicular to the first surface; and

an adhesive part for firmly contacting with the body of the test subject.

### BRIEF DESCRIPTION OF THE DRAWINGS

FIG. 1 is a diagram illustrating a block configuration of a sticker-type pulse wave sensor according to a first embodiment;

FIG. 2 is cross-sectional view illustrating an optical system of the sticker-type pulse wave sensor according to the first embodiment;

FIG. 3 is a front view illustrating the optical system of the sticker-type pulse wave sensor according to the first embodiment as viewed from a surface side that is to be firmly contacted with a test subject;

FIG. 4 is a diagram representing an example of a light propagation path of the sticker-type pulse wave sensor according to the first embodiment;

FIG. 5 is a diagram representing an example of a light propagation path of the sticker-type pulse wave sensor according to the first embodiment;

FIG. 6 is a block diagram illustrating a configuration by which to be executed by a control device according to the first embodiment;

FIG. 7 is a cross-sectional diagram illustrating an optical system of a sticker-type pulse wave sensor according to a second embodiment;

FIG. 8 is a front view illustrating the optical system of the sticker-type pulse wave sensor according to the second embodiment as viewed from a surface side that is to be firmly contacted with a test subject;

FIG. 9 is a diagram illustrating an example of a light propagation path of the sticker-type pulse wave sensor according to the second embodiment;

FIG. 10 is a diagram illustrating an example of a light propagation path of the sticker-type pulse wave sensor according to the second embodiment;

FIG. 11 is a diagram illustrating an example of a light propagation path of the sticker-type pulse wave sensor according to the second embodiment;

FIG. 12 is a diagram illustrating an example in which a sticker-type pulse wave sensor according to a third embodiment is affixed to an upper arm portion of the test subject;

FIG. 13 is a diagram illustrating an example in which the sticker-type pulse wave sensor according to the third embodiment is affixed to a clavicle portion area of the test subject;

FIG. 14 is a block diagram illustrating a configuration to be executed by a control device included in the sticker-type pulse wave sensor according to the third embodiment;

FIG. 15 is a conceptual diagram of a case in which a sticker-type pulse wave sensor according to a fifth embodiment is affixed to an upper arm portion.

FIG. 16 is a conceptual diagram of a case in which the sticker-type pulse wave sensor according to the fifth embodiment is affixed to a leg.

FIG. 17 is a conceptual diagram of a case in which multiple sticker-type pulse wave sensors according to a sixth embodiment are affixed to an upper arm;

FIG. 18 is a conceptual diagram of a case in which a sticker-type pulse wave sensor according and electrocardiogram-use electrodes according to a seventh embodiment are affixed to a soles of feet;

FIG. 19 is a block diagram illustrating a configuration to be executed by a control device included in the sticker-type pulse wave sensor according to the seventh embodiment;

FIG. 20 is a diagram illustrating an example of a procedure of a diagnostic service according to an eighth embodiment;

FIG. 21 is a diagram illustrating an example of a diagnostic system to be used with the diagnostic service according to the eighth embodiment;

FIG. 22 is a sequence diagram illustrating processing to be performed by the diagnostic system according to the eighth embodiment;

FIG. 23 is a cross-sectional view illustrating an optical system of a sticker-type pulse wave sensor according to a ninth embodiment;

FIG. 24 is a diagram illustrating a diagnostic system to be used with a diagnostic service according to the ninth embodiment;

FIG. 25 is a cross-sectional view illustrating an optical system of a sticker-type pulse wave sensor according to a tenth embodiment;

FIG. 26 is a diagram illustrating a block configuration of a sticker-type pulse wave sensor according to the tenth embodiment;

FIG. 27 is a diagram illustrating time-series information of blood pressure measured by the sticker-type pulse wave sensor according to the tenth embodiment;

FIG. 28 is a diagram illustrating a classification result by pattern-matching with respect to the time-series information of blood pressure according to the tenth embodiment;

FIG. 29 is a cross-sectional view illustrating an optical system of a sticker-type pulse wave sensor according to an eleventh embodiment;

FIG. 30 is a diagram illustrating an example in which the sticker-type pulse wave sensor according to the eleventh embodiment is affixed to the head of the test subject at a position behind the ear;

FIG. 31 is a diagram illustrating an example in which the sticker-type pulse wave sensor according to the eleventh embodiment is affixed to the left temple of the test subject;

FIG. 32 is a diagram illustrating an example in which the sticker-type pulse wave sensor according to the eleventh embodiment is affixed between the eyebrows of the test subject;

FIG. 33 is a diagram illustrating a result of measuring changes in blood pressure of a test subject and an example of an events that occurred with respect to the test subject;

FIG. 34 is a diagram illustrating an example in which only the events where the test subject talked are extracted by a cloud server according to the eleventh embodiment;

FIG. 35 is a diagram illustrating a personality model of a test subject;

FIG. 36 is a flowchart illustrating a process performed by a diagnostic system according to the eleventh embodiment;

FIG. 37 is a diagram illustrating a diagnostic system to be used with a diagnostic service according to a twelfth embodiment;

FIG. 38 is a flowchart illustrating processing performed by the diagnostic system according to the twelfth embodiment;

FIG. 39 is a flowchart illustrating processing regarding an alarm function of the diagnostic system according to the twelfth embodiment;

FIG. 40 is a diagram illustrating a configuration example of a project support system according to a thirteenth embodiment;

FIG. 41 is a sequence diagram illustrating processing performed in the project support system according to the thirteenth embodiment;

FIG. 42 is a flowchart illustrating a method of calculating an evaluation value for each participant in a participant evaluation server according to the thirteenth embodiment;

FIG. 43 is a diagram illustrating changes in blood pressure at a predetermined measurement part during a meeting of two participants in time series;

FIG. 44 is a diagram for describing a two-participant correlation coefficient calculation method performed by by a correlation calculation unit of the participant evaluation server according to the thirteenth embodiment;

FIG. 45 is a diagram illustrating matrixes of two-participant correlation coefficients for each measurement part calculated by the correlation calculation unit of the participant evaluation server according to the thirteenth embodiment;

FIG. 46 is a diagram illustrating a screen example illustrating evaluation values for each participant displayed by the evaluation value calculation unit of the participant evaluation server according to the thirteenth embodiment;

FIG. 47 is a flowchart illustrating a calculation technique of calculating a project evaluation value in the project evaluation server according to the thirteenth embodiment;

FIG. 48 is a diagram illustrating evaluation values for each meeting calculated by a calculation unit of the project evaluation server according to the thirteenth embodiment;

FIG. 49 is a diagram illustrating evaluation values of past projects stored in the project evaluation server according to the thirteenth embodiment in a matrix form; and

FIG. 50 is a diagram illustrating a screen example of advice for improvements output by the project evaluation server according to the thirteenth embodiment.

### DESCRIPTION OF THE EMBODIMENTS

It is desired to improve the detection accuracy of biological information such as a pulse wave, blood pressure, and the like of a body of a test subject.

According to the embodiments of the present disclosure, the accuracy in detecting biological information such as the pulse wave and the like can be enhanced because the shielding unit is provided between the light emitting unit and the light detecting unit on the circuit board that is flexible.

Herein, embodiments of a pulse wave sensor, a sphygmomanometer, a diagnostic system, a diagnostic method, a recording medium, and a meeting support system are described in detail with reference to the appended drawings.

### First Embodiment

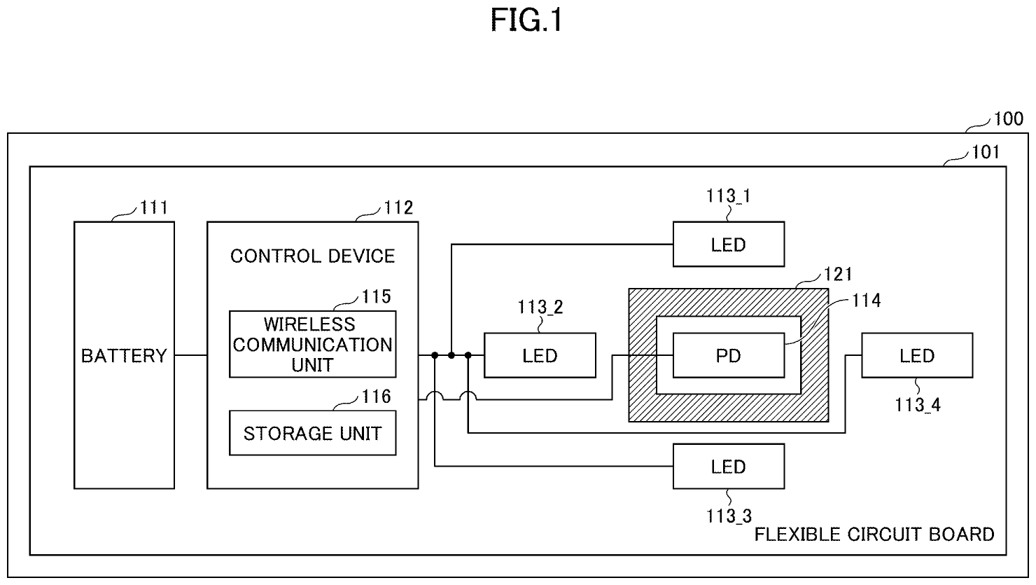

FIG. 1 is a diagram illustrating a block configuration of a sticker-type pulse wave sensor according to the first embodiment. As illustrated in FIG. 1, various components are mounted on a flexible printed circuit board 101 of a sticker-type pulse wave sensor 100. A battery 111, a control device 112, four LEDs, i.e., 113_1 to 113_4, and a PD 114 are mounted on the flexible printed circuit board 101 according to the present embodiment.

The sticker-type pulse wave sensor 100 according to the present embodiment is used for measuring pulse waves of a test subject (a person, for example). The sticker-type pulse wave sensor 100 according to the present embodiment includes a (non-illustrated) adhesive layer. Further, the person serving as the test subject removes the sticker-type pulse wave sensor 100 from (non-illustrated) release liner and affixes the sticker-type pulse wave sensor 100 to a point for measuring pulse waves.

As long as the aforementioned components can be mounted on the sticker-type pulse wave sensor 100, the sticker-type pulse wave sensor 100 may be any size. For example, the sticker-type pulse wave sensor 100 may have a rectangular shape with a height of 3 cm and a width of 5 cm. It is to be noted that the size of the sticker-type pulse wave sensor 100 is by no means limited to this shape, and may be of a size and shape suitable for the location to which the sticker-type pulse wave sensor 100 is to be affixed.

The sticker-type pulse wave sensor 100 is assumed to be disposable after the pulse wave measurement is performed (what is known as a single-use disposable). That is, after the person serving as the test subject affixes the sticker-type pulse wave sensor 100 to a part and performs a measurement for a predetermined period of time, the person can remove and dispose the sticker-type pulse wave sensor 100.

Consideration need not be paid to repeated use of the sticker-type pulse wave sensor 100 according to the present embodiment, and thus it is sufficient as long as the sticker-type pulse wave sensor 100 has durability sufficient for performing a pulse wave measurement once. Therefore, the sticker-type pulse wave sensor 100 is constituted by a thin film.

The flexible printed circuit board (FPC) 101 is a type of printed circuit board and includes a flexible cable (example of wiring) connecting the control device 112 and the LEDs together 113_1 to 113_4 together and includes a flexible cable (example of wiring) connecting the control device 112 and the PD 114 together.

The flexible printed circuit board (FPC) 101 is a flexible circuit board that can change shape in accordance with changes in movement on a surface of a test subject due to body movement and the like and can maintain electrical characteristics even when the shape changes.

The battery 111 is a power source that provides power to the control device 112, the LEDs 113_1 to 113_4, the PD 114, and the like while a pulse wave is being measured, and is for example a button battery. The sticker-type pulse wave sensor 100 according to the present embodiment conceivably measures one day's worth of pulse waves of a test subject, for example. In such a case, the battery 111 according to the present embodiment is sufficient as long as the battery 111 can provide power to the control device 112, the LEDs 113_1 to 113_4, the PD 114, and the like for a day or longer.

Also, the sticker-type pulse wave sensor 100 is single-use disposable, and thus the battery 111 does not need to be rechargeable. Therefore, the sticker-type pulse wave sensor 100 need not have an external port for charging and can be a sealed configuration owing to a resin thereof that is water resistant.

The control device 112 includes a wireless communication unit 115 and a storage unit 116 and controls the entirety of the sticker-type pulse wave sensor 100. For example, the control device 112 individually controls the emitting of the four LEDs 113_1 to 113_4.

The wireless communication unit 115 is a configuration for performing wireless communication with an external device. As a method of wireless communication of the wireless communication unit 115 according to the present embodiment, it is conceivable to use a wireless communication scheme such as Wi-Fi (registered trademark), Bluetooth (registered trademark, and the like. Is should be noted that the external device with which the wireless communication unit 115 communicates may be a communication device or the like that is owned by the test subject.

The storage unit 116 is used for storing detection information indicating the detection results transmitted from the PD 114 as well for storing the programs that is executed by the control device 112. It is sufficient as long as the storage unit 116 is a non-volatile recording medium capable of reading and writing.

The four light emitting diodes (LEDs) 113_1 to 113_4 emit light with a wavelength of close to 520 nm and a luminous intensity of close to 100 cd in accordance with a control from the control device 112. It should be noted that the wavelength and the luminous intensity at which the LEDs 113_1 to 113_4 emit light is an example and is by no means limited to the aforementioned wavelength and luminous intensity. It is sufficient as long as the wavelength and the luminous intensity are such that measurement of the pulse wave or the like of the test subject is measurable. In the present embodiment, although an example in which LEDs are used as an example of the light emitting unit is described, the LEDs are by no means a limitation, and any configuration may be used as long as the configuration is configured to emit light.

The PD 114 (example of the light detecting unit) detects light reflected (propagated) in the test subject. The PD 114 according to the present embodiment uses a component in which the IC for control of the preamplifier, A/D converter, storage unit, and the like are built on the same circuit board. This is because when there is wiring between the photodiode and the amplifier, there is a possibility that the detection accuracy of the pulse waves will decrease due to noise generated by the wiring. As such, in the present embodiment, the PD including the IC for control are referred to as the PD 114.

The PD 114 according to the present embodiment transmits a detection value indicative of the detection result to the control device 112 as a digital signal. Although any technique may be used as the transmission technique, it is conceivable to perform transmission based on a standard such as I2C or the like.

The control device 112 also functions as an LED driver by performing control such that the four LEDs 113_1 to 113_4 are individually caused to emit light periodically in time division by power provided from the battery 111.

The light emitted from each of the LEDs 113_1 to 113_4 incidents a measurement part of the to-be-detected body and then, after reflecting and scattering repeatedly in the measurement part, the light is transmitted towards the sticker-type pulse wave sensor 100. Then, the PD 114 measures the transmitted light and outputs a signal indicating the detection result to the control device 112.

Incidentally, oxyhemoglobin exists in the blood of the artery of the test subject and the oxyhemoglobin has a characteristic of absorbing incident light. Therefore, the control device 112 according to the present embodiment measures the pulse wave signal based on light detected by the PD 114, by measuring in time series, the blood flow amount blood flow amount that changes in accordance with heart pulsations (volume change of artery).

As described above, since it is necessary for the PD 114 to detect light reflected inside the test subject, a shielding layer 121 is provided between the PD 114 and the LEDs 113_1 to 113_4 in the present embodiment.

The shielding layer 121 is a shielding member formed such that light is blocked in order to inhibit light emitted from the LEDs 113_1 to 113_4 from directly incidenting on the PD 114. A conceivable example of the shielding member is a silicone resin in which black carbon is mixed. Next, an optical system of the sticker-type pulse wave sensor 100 is described.

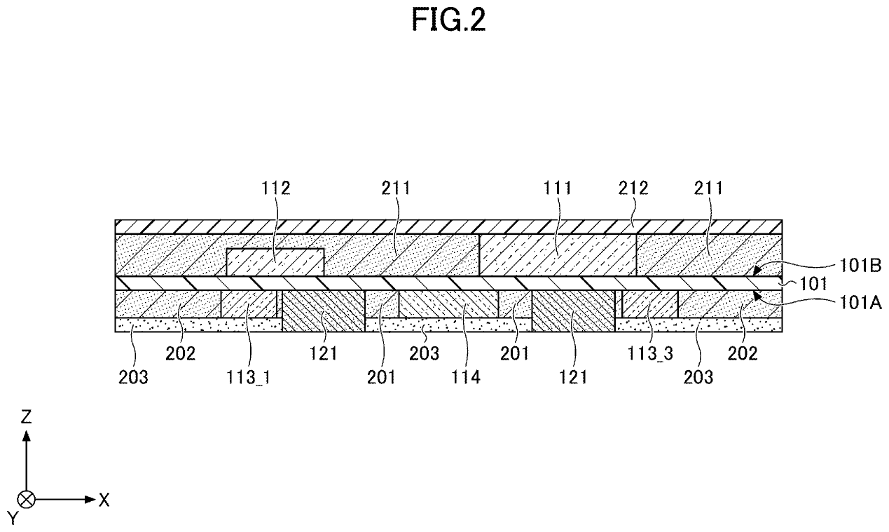

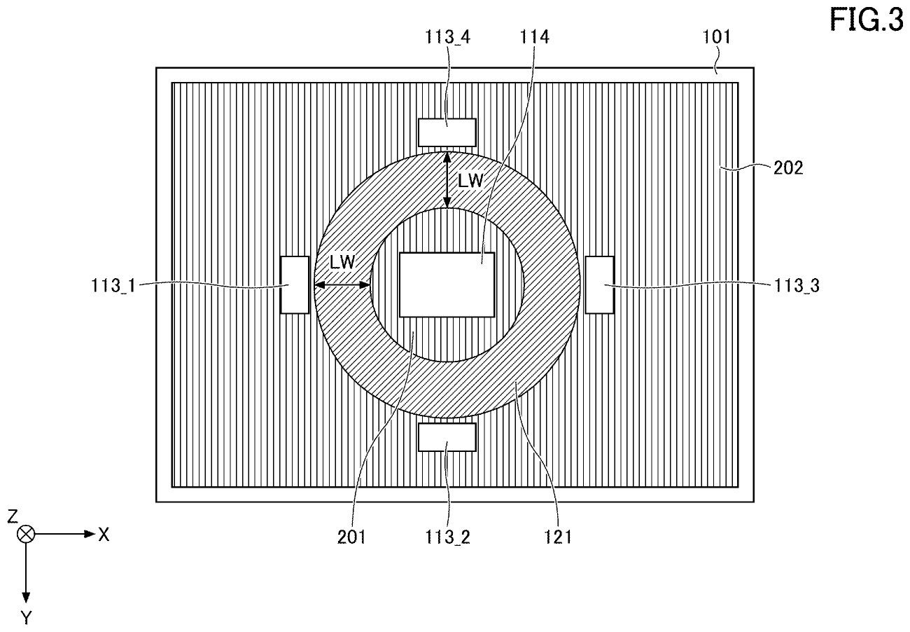

FIG. 2 is a cross-sectional view illustrating an optical system of the sticker-type pulse wave sensor 100 according to the present embodiment. FIG. 3 is a front view illustrating the optical system of the sticker-type pulse wave sensor 100 according to the present embodiment as viewed from a surface side that is to be firmly contacted with a test subject.

As illustrated in FIG. 2, devices are provided on a first surface 101A and on a second surface 101B of the flexible printed circuit board 101 of the sticker-type pulse wave sensor 100. The first surface 101A is on the side (on the negative Z-axis side) of the flexible printed circuit board 101 of the sticker-type pulse wave sensor 100 to be firmly contacted with the test subject, whereas the second surface 101B (on the positive Z-axis side) is facing away from that side to be firmly contacted with the test subject.

The battery 111 and the control device 112 are provided on the second surface 101B, i.e., on the surface facing away from the side to be firmly contacted with the test subject (on the positive Z-axis side). Further, in order to flatten any irregularities caused by the battery 111 and the control device 112, a silicone resin layer 211 is provided on the second surface 101B.

Furthermore, the outer side of the silicone resin layer 211 is provided with a cover layer 212. The cover layer 212 is a flexible member that inhibits light from entering inside the sticker-type pulse wave sensor 100. Examples of the cover layer 212 include an aluminum metalized film and the like.

The first surface 101A is a surface on the side to be firmly contacted with the test subject (on the negative Z-axis side). The PD 114 is provided at the center of the first surface 101A and other components are around the PD 114.

As illustrated in FIGS. 2 and 3, a PD-use attachment 201 is provided such that the PD-use attachment is in contact with the PD 114. The shielding layer 121 is provided on the outer side of the PD-use attachment 201. Also, the LEDs 113_1, 113_2, 113_3, and 113_4 are arranged on the outer side of the shielding layer 121.

In the example illustrated in FIG. 3, each of the LEDs 113_1 to 113_4 is placed at a distance of, for example, 10 mm from the PD 114. It is to be noted that the distance between the PD 114 and LEDs 113_1 to 113_4 is merely given as an example, and the distance may be made different in accordance with measurement part or the like of the test subject.

Furthermore, an attachment 202 is provided on the outer side of the LEDs 113_1 and 113_2.

Furthermore, the PD-use attachment 201 and the attachment 202 are provided in order to flatten any irregularities formed by the placement of the PD 114 and the LEDs 113_1 and 113_2 on the first surface 101A. It is sufficient as long as the PD-use attachment 201 and the attachment 202 are flexible members. It is conceivable to use a silicone resin or the like as the PD-use attachment 201 and the attachment 202. As the silicone resin, it is possible to consider applying a commonly-used cloudy material to the PD-use attachment 201 and the attachment 202. By doing so, the absorption of light can be inhibited as much as possible.

Furthermore, both surfaces of the PD-use attachment 201 are treated such that both surfaces function as mirrors. As the treating method, the film forming of a vapor-deposited aluminum film is conceivable. Aluminum film formation is not limited to vapor deposition, and a low cost manufacturing technique such as plating may be used.

In the present embodiment, by imparting the PD-use attachment 201 with a mirror function (light can be reflected), light arriving at the PD-use attachment 201 can be reflected again toward the test subject without any absorption by the PD-use attachment 201. The light incident on the test subject is reflected again inside the test subject. In other words, the incidenting of light on the test subject and the reflecting by the PD-use attachment 201 leading up to the light entering the PD 114 are repeated. That is, the amount of light arriving at the PD 114 can be increased. By doing so, the amount of light detected by the PD 114 can be increased, and thus the accuracy in measuring the pulse wave can be increased.

By imparting the PD-use attachment 201 according to the present embodiment with a mirror function, the amount of light incident on the PD 114 can be increased, and thus the electrical amplifier settings of the PD 114 can be made smaller. By doing so, the noise generated by detection of the signal of the PD 114 can be reduced.

The mirror function of the PD-use attachment 201 according to the present embodiment is merely an example, and thus the PD-use attachment 201 need not necessarily have a mirror function.

Also, an adhesive layer 203 may be provided on the test subject side such that the PD 114, the LEDs 113_1 and 113_2, the PD-use attachment 201, and the attachment 202 arranged on the first surface 101A are covered.

The adhesive layer 203 serves as a member for firmly contacting the sticker-type pulse wave sensor 100 with the test subject. An acrylic adhesive is conceivable as the material of the adhesive layer 203. Given that the adhesive function is to be firmly contacted with skin, it is also necessary to have a function that does not damage the skin. A material that has good breathability and next to no irritation on the skin such as sticking plaster is selected as the adhesive layer 203. Although an adhesive layer with a thickness of 10 μm exhibits functionality, the adhesive layer 203 is set to have a thickness of approximately 100 μm in order to deal with irregularities on the surface or the like.

The adhesive layer 203 is a member that is transparent to light emitted from the LEDs 113_1 and 113_2. The adhesive layer 203 is not limited to a transparent member, and although a cloudy member may be used, it is preferably to keep the light attenuation coefficient low as much as possible. By doing so, the sticker-type pulse wave sensor 100 according to the present embodiment can suppress a reduction in the amount of light that is incident on the PD 114, and thus the accuracy in detecting the pulse wave can be enhanced.

The shielding layer 121 is provided on the first surface 101A and is situated between the PD 114 and the LEDs 113_1 to 113_4 such that the shielding layer 121 protrudes beyond the PD 114 and the LED 113_1 to 113_4 in a direction (Z-axis direction perpendicular to the first surface 101A. Specifically, the length of the shielding layer 121 in the Z-axis direction is substantially the same as a length obtained by adding thickness of the adhesive layer 203 to the length of the PD 114 and the LEDs 113_1 to 113_4 in the Z-axis direction. By doing so, the light emitted from the LEDs 113_1 to 113_4 can be suppressed from incidenting on the PD 114 via the adhesive layer 203 without crossing into the test subject.

Also, the shielding layer 121 has a width in the X-axis direction and the Y-axis direction to an extent that the light incident from the LEDs 113_1 to 113_4 does not reach the PD 114. For example, the width LW of the shielding layer 121 may be from 2 mm to 3 mm.

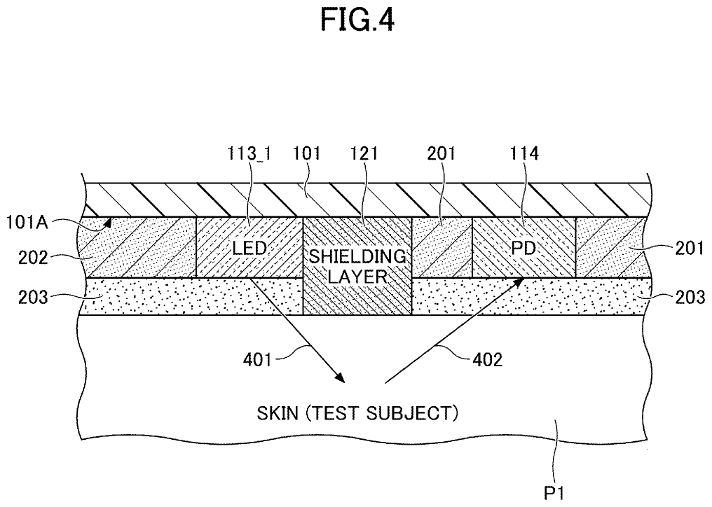

FIG. 4 is a diagram representing an example of a light propagation path of the sticker-type pulse wave sensor 100 according to the present embodiment. In the example illustrated in FIG. 4, a case is illustrated in which the sticker-type pulse wave sensor 100 is firmly contacted with the skin (measurement part P1) of the test subject, without there being any change in the shape of the skin (measurement part P1) due to body movement or the like from the time when the sticker-type pulse wave sensor 100 was affixed.

As illustrated in FIG. 4, in a case where the sticker-type pulse wave sensor 100 is firmly contacting the skin, direct light can be suppressed from propagating from the LED 113_1 to the PD 114 because the shielding layer 121 has the aforementioned configuration.

Therefore, the light emitted from the LED 113_1 travels along a path 401 and incidents on a measurement part P1 of the test subject. The incident light is reflected inside the measurement part P1 of the test subject. Then, the PD 114 receives the light traveling along a path 402 after having been reflected inside the measurement part P1 of the test subject.

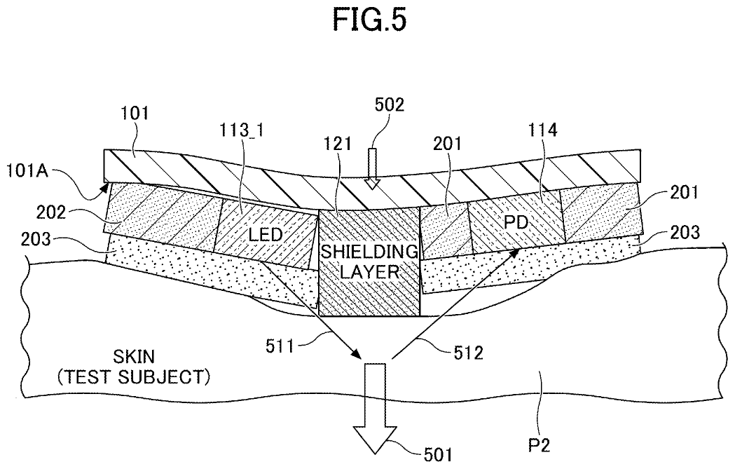

FIG. 5 is a diagram representing an example of a light propagation path of the sticker-type pulse wave sensor 100 according to the present embodiment. In the example illustrated in FIG. 5, a situation is depicted in which the skin (measurement part P2) of the test subject changed shape due to body movement or the like from the time when the sticker-type pulse wave sensor 100 was affixed.

As described above, the flexible printed circuit board 101, the PD-use attachment 201, the attachment 202, and the adhesive layer 203 of the sticker-type pulse wave sensor 100 are flexible. Also, the adhesive layer 203 of the sticker-type pulse wave sensor 100 is affixed to an entire surface of the sticker-type pulse wave sensor 100 except for where the shielding layer 121 is. Therefore, as illustrated in FIG. 5, the shape of the sticker-type pulse wave sensor 100 changes such that the shape follows body movement of the skin (measurement part P2) of the test subject whose shape changed.

FIG. 5 is an example in which the skin (measurement part P2) of the test subject moves in the direction of arrow 501. In such a case, if the pulse wave sensor is a conventional type which is not flexible, a void forms between the skin and the pulse wave sensor. Then, a situation occurs where direct light incidents on the PD from the LED via the void.

In order to address this, the sticker-type pulse wave sensor 100 according to the present embodiment changes to a shape protruding in the direction of arrow 502 such that the sticker-type pulse wave sensor 100 follows along the skin (measurement part P2) of the test so as not to form a void between the shielding layer 121 and the skin (measurement part P2) of the test subject. By doing so, direct light can be suppressed from propagating from the LED 113_1 to the PD 114.

Therefore, the light emitted from the LED 113_1 travels along a path 511 and incidents on a measurement part P2 of the test subject. The incident light is reflected inside the measurement part P2 of the test subject. Then, the PD 114 receives the light traveling along a path 512 after having been reflected inside the measurement part P2 of the test subject.

By providing the sticker-type pulse wave sensor 100 according to the present embodiment with the aforementioned configuration, the light emitted from the LED 113_1 gets reflected by the measurement part of the test subject, and thus the PD 114 can receive this reflected light. Then the control device 112 measures the pulse wave based on the received light.

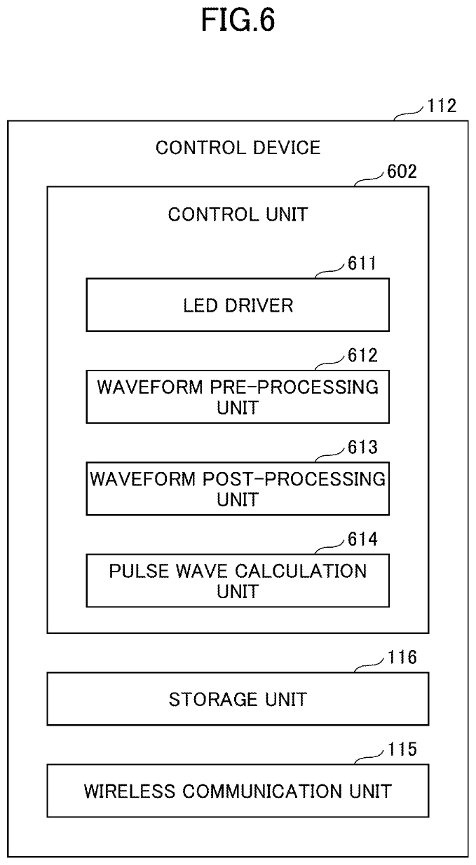

FIG. 6 is a block diagram illustrating a configuration to be executed by the control device 112. As illustrated in FIG. 6, the control device 112 includes the wireless communication unit 115, the storage unit 116, and a control unit 602.

The storage unit 116 stores therein a program to be executed by the control unit 602. The control unit 602 implements the various configurations by executing programs and the like stored in the storage unit 116.

The control unit 602 serves as an LED driver 611, a waveform pre-processing unit 612, a waveform post-processing unit 613, and a pulse wave calculation unit 614 by executing programs stored in the storage unit 116.

The LED driver 611 is a driver for controlling the LEDs 113_1 to 113_4. The LED driver 611 performs control such that the four LEDs 113_1 to 113_4 are individually caused to emit light periodically in time division.

The LED driver 611 repeats light emission and light non-emission at a timing of approximately 1 kHz as the light emission timing of the LEDs 113_1 to 113_4.

Also, a method for obtaining a detection value difference at a timing synchronized with that repetition, what otherwise known as a method of a lock-in amplifier, is adopted by the amplifier of the PD 114.

The waveform pre-processing unit 612 generates signals to control the LEDs 113_1 to 113_4 by using the LED driver 611 and performs pre-processing with respect to the generated signals. As this pre-processing, smoothing, filtering such as noise reduction or the like, and so on is performed for example.

The waveform post-processing unit 613 performs post-processing with respect to detection information input from the PD 114, and after doing so, stores the detection information into the storage unit 116. As this post-processing, smoothing, filtering such as noise reduction or the like, and so on is performed for example.

The pulse wave calculation unit 614 calculates, based on detection information stored in the storage unit 116, a pulse wave (an example of information regarding the pulse wave) by detecting a volume of the artery of the test subject which changes in accordance with pulsations.

Although an example is given in which the sticker-type pulse wave sensor 100 calculates a pulse wave in the present embodiment. The pulse wave may be calculated by an external device that is connected to the sticker-type pulse wave sensor 100. In this case, features (an example of information regarding a pulse wave) that is necessary for calculation of the pulse wave by the external device may be extracted by the sticker-type pulse wave sensor 100. Then, the sticker-type pulse wave sensor 100 may transmit the feature to the external device (for example, a mobile terminal of the test subject). In this case, the program for calculating biological information such as the pulse wave of the test subject, based on this feature, is stored in the external device.

By providing the sticker-type pulse wave sensor 100 according to the present embodiment with the aforementioned configuration, the light emitted from the LED 113_1 to 113_4 can be suppressed from directly incidenting on the PD 114, and thus the accuracy in measuring the pulse wave can be enhanced.

Since the flexible printed circuit board 101 and the like of the sticker-type pulse wave sensor 100 according to the present embodiment are flexible, the optical system (for example, the PD 114 and the LEDs 113_1 to 113_4) can always maintain firm contact with the skin via the adhesive layer 203. By doing so, the sticker-type pulse wave sensor 100 can suppress the effect of fluctuating factors caused at the interface of the skin and air. Therefore, the sticker-type pulse wave sensor 100 can enhance the accuracy in detecting pulse waves can be enhanced.

Since the light that is detected by the sticker-type pulse wave sensor 100 according to the present embodiment is only light that propagated in the body of the test subject, external environmental effects can be suppressed, and thus minute changes in the detected light can be detected. Therefore, the light that is detected by the sticker-type pulse wave sensor 100 enables highly accurate detection of the waveform of pulse waves.

It is envisaged that the sticker-type pulse wave sensor 100 according to the present embodiment will perform continuous measurements over a long-period of time such as 24 hours. The shielding layer 121 of the sticker-type pulse wave sensor 100 is placed such that the shielding layer 121 surrounds the PD 114. The shielding layer 121 is thicker than the flexible printed circuit board 101 and the elastic modulus is high. Furthermore, the shielding layer 121 is elastic to the extent that the shielding layer 121 can conform with the shape of the specimen. The sticker-type pulse wave sensor 100 according to the present embodiment can maintain a state where the shielding layer 121 is abutted against the skin even when a depression forms due to body movement of the test subject. With this, highly accurate measurement of pulse waves can be maintained even when body movement occurs.

In the sticker-type pulse wave sensor 100 according to the present embodiment, the shielding layer 121 is provided between the LEDs 113_1 to 113_4 and the PD 114, and is on the same plane as the surface contacting with the test subject. Also, the adhesive layer 203 is provided in a region other than were the shielding layer 121 is. Therefore, peeling of the sticker-type pulse wave sensor 100 from the skin of the test subject can be suppressed.

Also, since the sticker-type pulse wave sensor 100 according to the present embodiment is provided with the aforementioned configuration, use as a disposable is also possible. In other words, although the durability of the sticker-type pulse wave sensor 100 is low, an extremely soft material can be adopted. In doing so, the sticker-type pulse wave sensor 100 can conform with the shape of the test subject, and thus the test subject is unlikely experience any discomfort.

With the sticker-type pulse wave sensor 100, it is assumed that the adhesive layer 203 too will only be used once, and thus a member having an adhesive force strong enough so that the adhesive surface can withstand skin cells, detritus, and the like of the test subject.

### Second Embodiment

In the first embodiment, an example is described in which an adhesive layer 203 having the smallest possible light attenuation coefficient is used. However, the adhesive layer may have a shielding property. Thus, in the second embodiment, an example is described in which the shielding layer also functions as an adhesive layer.

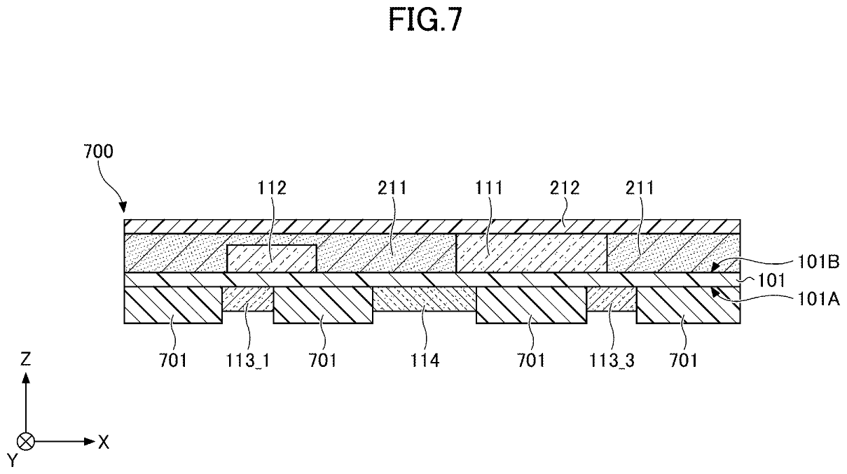

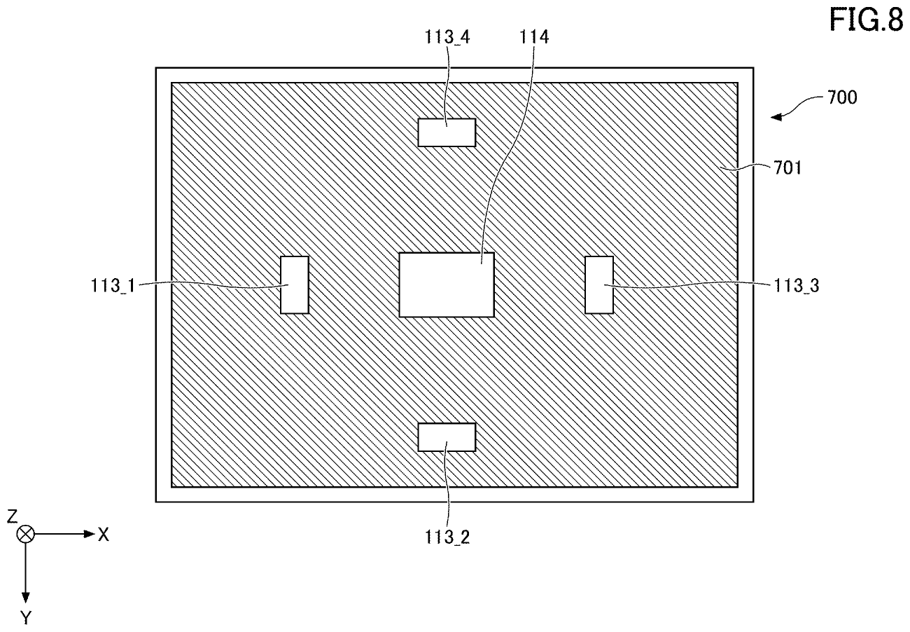

FIG. 7 is a cross-sectional diagram illustrating an optical system of a sticker-type pulse wave sensor 700 according to the present embodiment. FIG. 8 is a front view illustrating the optical system of the sticker-type pulse wave sensor 700 according to the present embodiment as viewed from a surface side that is to be firmly contacted with the test subject. The components that are the same as those of the first embodiment are denoted by the same reference numerals and descriptions are omitted.

The PD 114 is provided at the center of the first surface 101A and other components are arranged around the PD 114.

As illustrated in FIGS. 7 and 8, a shielding layer 701 is provided around the PD 114. Also, voids are provided at four locations in the shielding layer 701. The LEDs 113_1, 113_2, 113_3, and 113_4 are arranged in the voids at these four locations.

The shielding layer 701 is a shielding member formed such that light is blocked in order to inhibit light emitted from the LEDs 113_1 to 113_4 from directly incidenting on the PD 114. A conceivable example of the shielding member is a silicone resin in which black carbon is mixed. Furthermore, an end surface, of the shielding layer 701, firmly contacting with the test subject is formed as an adhesive part. In the present embodiment, the entire surface of the shielding layer 701 of FIG. 8 functions as the adhesive part. By doing so, the sticker-type pulse wave sensor 700 can be affixed to the test subject.

In the present embodiment, although a case is described in which the end surface of the shielding layer 701 firmly contacting with the test subject is formed as the adhesive part, an adhesive layer may be provided on the end surface of the shielding layer 701. In this case, a black carbon or the like is mixed in the adhesive layer, and by doing so, the adhesive layer can be formed such that light can be blocked, thereby imparting a light blocking function. In other words, the adhesive layer also functions as a shielding layer.

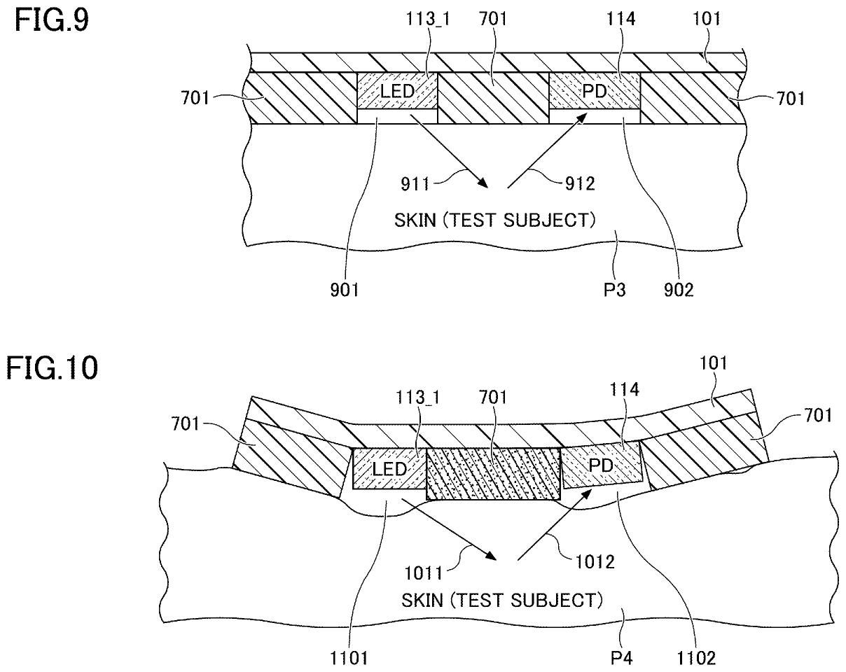

FIG. 9 is a diagram illustrating an example of a light propagation path of the sticker-type pulse wave sensor 700 according to the present embodiment. In the example illustrated in FIG. 9, the sticker-type pulse wave sensor 700 is firmly contacted with the skin (measurement part P3) without there being any change in shape of the skin (measurement part P3) due to body movement or the like from the time when the sticker-type pulse wave sensor 700 was affixed.

As illustrated in FIG. 9, in a case where the sticker-type pulse wave sensor 100 is firmly contacting with the skin, each of the shielding layers 701 is firmly contacted with the skin (measurement part P3), and thus a void 901 exists on the surface of the LED 113_1 and a void 902 also exists on the surface of the PD 114. As illustrated in FIG. 9, since the shielding layers 701 are firmly contacted with the skin, the configuration is such that direct light from the LED 113_1 does not reach the PD 114.

Therefore, light emitted from the LED 113_1 incidents on a measurement part P3 of the test subject along a path 911. The incident light is reflected inside the measurement part P3 of the test subject. Then, the PD 114 receives the light traveling along a path 912 after having been reflected inside the measurement part P3 of the test subject.

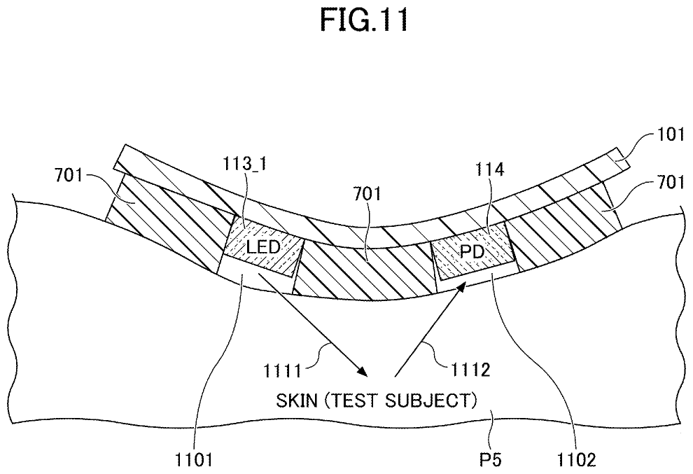

FIG. 10 is a diagram illustrating an example of a light propagation path of the sticker-type pulse wave sensor 700 according to the present embodiment. In the example of FIG. 10, a situation is depicted in which the skin (measurement part P4) of the test subject changed due to body movement or the like from the time when the sticker-type pulse wave sensor 700 was affixed.

As described above, the flexible printed circuit board 101 and the shielding layer 701 of the sticker-type pulse wave sensor 700 are flexible. Also, the shielding layer 701 of the sticker-type pulse wave sensor 700 is affixed to an entire surface of the sticker-type pulse wave sensor 700 except for where the PD 114 and the LEDs 113_1 to 113_4 are. Therefore, as illustrated in FIG. 10, the shape of the sticker-type pulse wave sensor 700 changes such that the shape conforms with the body movement of the skin (measurement part P4) of the test subject whose shape changed. A void 1101 on the surface of the LED 113_1 and a void 1102 on the surface of the PD 114 are substantially the same as those in FIG. 9.

The sticker-type pulse wave sensor 700 according to the present embodiment changes shape such that the sticker-type pulse wave sensor 700 follows the body movement of the skin (measurement part P4) of the test subject so as not to form a void between the shielding layer 701 and the skin (measurement part P4) of the test subject. By doing so, direct light can be suppressed from propagating from the LED 113_1 to the PD 114.

Therefore, the light emitted from the LED 113_1 travels along a path 1011 and incidents on a measurement part P4 of the test subject. The incident light is reflected inside the measurement part P4 of the test subject. Then, the PD 114 receives the light traveling along a path 1011 after having been reflected inside the measurement part P4 of the test subject.

FIG. 11 is a diagram illustrating an example a light propagation path of the sticker-type pulse wave sensor 700 according to the present embodiment. The example illustrated in FIG. 11 depicts a situation in which the sticker-type pulse wave sensor 700 is affixed to skin (measurement part P5) of the test subject having a recessed shape. The portion of the test subject that has a recessed shape is, for example, in vicinity of the clavicle.

As described above, the flexible printed circuit board 101 and the shielding layer 701 of the sticker-type pulse wave sensor 700 are flexible. Also, the shielding layer 701 of the sticker-type pulse wave sensor 700 is affixed to an entire surface of the sticker-type pulse wave sensor 700 except for where the PD 114 and the LEDs 113_1 to 113_4 are. Therefore, as illustrated in FIG. 11, even when the skin (measurement part P5) of the test subject is a recessed shape, the sticker-type pulse wave sensor 700 can be affixed such that the sticker-type pulse wave sensor 700 conforms with that shape. The void 1101 on the surface of the LED 113_1 and the void 1102 of the surface of the PD 114 are substantially the same as those in FIG. 9.

The sticker-type pulse wave sensor 700 according to the present embodiment changes shape such that the sticker-type pulse wave sensor 700 follows the body movement of the skin (measurement part P5) of the test subject so as not to form a void between the shielding layer 701 and the skin (measurement part P5) of the test subject. By doing so, direct light can be suppressed from propagating from the LED 113_1 to PD 114.

Therefore, the light emitted from the LED 113_1 travels along path 1111 and incidents on a measurement part P5 of the test subject. The incident light is reflected inside the measurement part P5 of the test subject. Then, the PD 114 receives the light traveling along a path 1112 after having been reflected inside the measurement part P5 of the test subject.

In the present embodiment, by providing the aforementioned configuration, the shielding unit also firmly contacts with the skin the test subject, and substantially the same effects as in the first embodiment can be obtained. Therefore, even when the measurement part is in a recessed shape, the sticker-type pulse wave sensor 700 can perform highly-accurate measurements of pulse waves.

### Third Embodiment

In the aforementioned embodiments, a case is described where a pulse wave is measured in the sticker-type pulse wave sensor. However, embodiments described above by no means limit the technique to a technique of measuring only pulse waves. As such, in the third embodiment, a case is described in which blood pressure is measured based on the pulse waves.

A sticker-type pulse wave sensor according to the third embodiment has substantially the same shape and substantially the same optical system as the sticker-type pulse wave sensor according to the first embodiment and the second embodiment, and thus such descriptions are omitted. The sticker-type pulse wave sensor according to the third embodiment differs from the sticker-type pulse wave sensor according to the first embodiment and the second embodiment in that the sticker-type pulse wave sensor of the first embodiment is a program that is executed in the control device.

Incidentally, nowadays, wristwatch-type biological measurement devices are prevalent in use. Even in these biological measurement devices, there is a function that estimates blood pressure. In these biological measurement devices, it is often the case that blood pressure estimation involving the use of a pulse wave propagation time method is used. Since these biological measurement devices are worn around the wrist, the relative height relationship with the heart is not stable. Consequently, the blood pressure measurements performed by these biological measurement devices are prone to error because the height relationship with the heart is not stable. In order to inhibit such errors, it is often the case that a measurement technique is used in which the arm, i.e. the measurement part, on which the biological measurement device is worn, is raised to the height of the chest, to perform measurement and the like, for example.

In contrast to this, the sticker-type pulse wave sensor according to the present embodiment involves a technique in which the sticker-type pulse wave sensor is attached to the measurement part of the test subject. Therefore, it is easy to attach the sticker-type pulse wave sensor according to a location that is substantially the same height as the heart in order to perform the blood pressure measurement by the sticker-type pulse wave sensor of the present embodiment. As such, an example of the attachment position of the sticker-type pulse wave sensor according to the third embodiment is described.

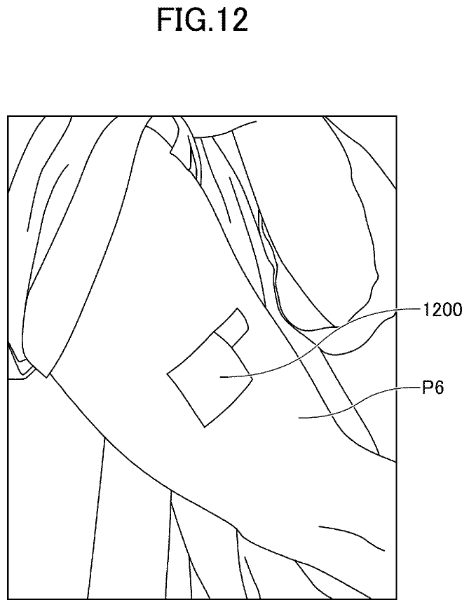

FIG. 12 is a diagram illustrating an example in which a sticker-type pulse wave sensor 1200 according to the present embodiment is affixed to an upper arm portion. As illustrated in FIG. 12, the sticker-type pulse wave sensor 1200 is attached an upper arm portion P6 of the test subject. Since the height of the upper arm and the height of the heart portion are substantially the same, the sticker-type pulse wave sensor 1200 according to the present embodiment is capable of blood pressure measurements with little error of estimation without any constraints imposed on the test subject with respect to movement. In other words, the sticker-type pulse wave sensor 1200 functions as a sphygmomanometer.

Furthermore, the affixing position when blood pressure measurement is to be performed in the sticker-type pulse wave sensor 1200 according to the present embodiment is by no means limited to the upper arm portion, and thus other body portions may be used.

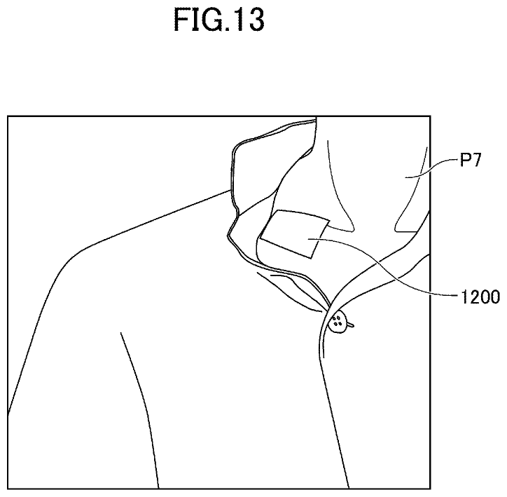

FIG. 13 is a diagram illustrating an example in which the sticker-type pulse wave sensor 1200 according to the present embodiment is affixed a clavicle portion area of the test subject. As illustrated in FIG. 13, the sticker-type pulse wave sensor 1200 is affixed to a clavicle portion area P7. The sticker-type pulse wave sensor 1200 is formed in a size (3 cm×5 cm, for example) such that the sticker-type pulse wave sensor 1200 is settable in vicinity of the subclavian artery of a test subject (person).

Since the height of the clavicle portion area and the height of the heart portion are substantially the same, the sticker-type pulse wave sensor 1200 according to the present embodiment is capable of blood pressure measurements with little error of estimation and without any constraints imposed on the test subject with respect to movement.

Incidentally, the subclavian artery is at the upper portion of the clavicle. The subclavian artery is an artery extending from the heart, which is beneath the ribs, and is located closer to the skin surface than the ribs are. Therefore, the pulse wave of the artery appears clearly. Moreover, since the movement of the legs and arms has little effect on pulse waves, the pulse waves are unlikely to be affected by body movements. Therefore, by affixing the sticker-type pulse wave sensor 1200 to the surface of the clavicle, pulse waves with little noise can be detected, and thus accuracy of blood pressure estimation can be increased.

Since the sticker-type pulse wave sensor 1200 according to the present embodiment is flexible, even when the portion of the test subject such as the clavicle portion area has a recessed shape, the sticker-type pulse wave sensor 1200 can be affixed such that the sticker-type pulse wave sensor 1200 conforms to the shape. Since the clavicle portion area is unlikely to move due to body movements, a signal with little noise can be detected with high accuracy, and thus pulse waves and blood pressure estimations can be performed with high accuracy.

Next, a configuration for measuring blood pressure and the like by the sticker-type pulse wave sensor 1200 according to the present embodiment is described.

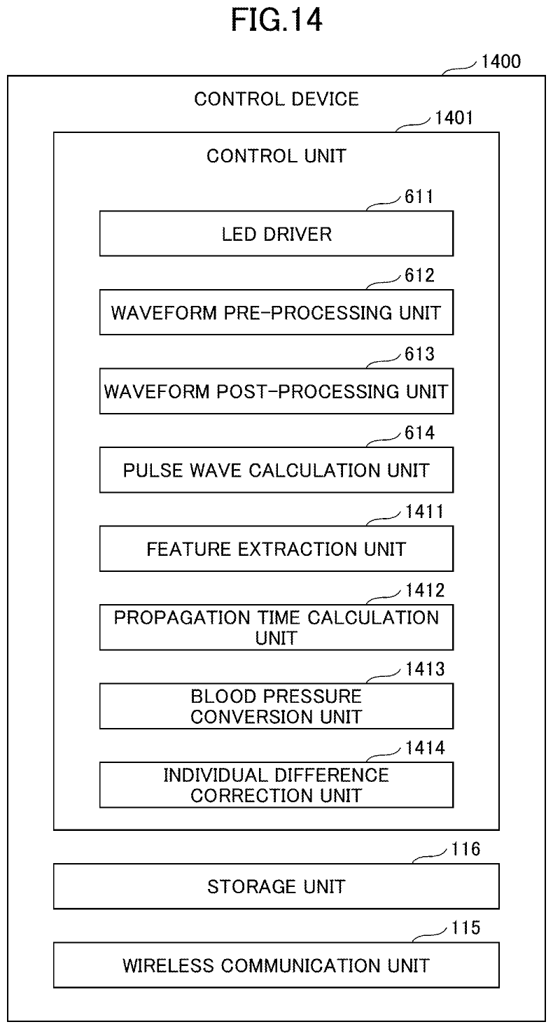

FIG. 14 is a block diagram illustrating a configuration to be executed by a control device 1400 included in the sticker-type pulse wave sensor 1200 according to the present embodiment. The sticker-type pulse wave sensor 1200 according to the present embodiment is an example in which the sticker-type pulse wave sensor is equipped with the control device 1400 instead of the control device 112 according to the aforementioned embodiments. The other components are substantially the same as the aforementioned embodiments so descriptions are omitted. Also, the components that are the same as those of the aforementioned embodiments are denoted by the same reference numbers and descriptions are omitted.

The control device 1400 differs from the control device 112 according to the aforementioned embodiments in that the control device 1400 includes a control unit 1401 with processing different from that of the control unit 602.

The control unit 1401 includes a feature extraction unit 1411, a propagation time calculation unit 1412, a blood pressure conversion unit 1413, and an individual difference correction unit 1414, in addition to including the same components as those in the control unit 602.

The feature extraction unit 1411 extracts, from a pulse wave, a feature for estimating blood pressure. Peaks of a percussion wave (PW) and a tidal wave (TW) exist in the pulse wave. The PW is depicted as a peak of the wave caused by the beat of the heart. Also, the TW is depicted as a beat reflected by a peripheral blood vessel of the leg. Furthermore, characteristic rises and falls, such as a dip (ND) that occurs when the aortic valve closes, are present in the pulse wave. Therefore, the feature extraction unit 1411 extracts peaks such as PW and TW and characteristic rises and falls, such as a dip, as features.

The propagation time calculation unit 1412 calculates a pulse wave propagation time based on the features of the pulse wave extracted by the feature extraction unit 1411. The pulse wave propagation time is a time necessary for pulse pressure waveform to propagate the length of the arterial tree.

The blood pressure conversion unit 1413 converts the calculated pulse wave propagation time to blood pressure. There is a correlative relationship between pulse wave propagation time and blood pressure. Therefore, the blood pressure conversion unit 1413 according to the present embodiment converts the pulse wave propagation time to blood pressure.

Any method may be used as the blood pressure conversion technique, one example being the technique introduced in the publication: Satomi Suzuki and Koji Oguri, “Cuffless Blood Pressure Estimation with Photoplethysmograph Signal by Classifying on Account of Cardiovascular Characteristics of Old Aged Patients”, The Transactions on Electrical and Electronic Engineering. C, A Publication of Electronics, Information and System Society, Vol. 130, Issue 2, pp. 261 to 266, 2010.

The individual difference correction unit 1414 performs correction based on individual differences with respect to the blood pressure converted by the blood pressure conversion unit 1413. Any techniques can be used for the correction techniques, one example being a correction that his performed by using an AI learning model trained based on parameters depicting an individual (for example, age, height, weight, and so on).

### Variation of Third Embodiment

In the third embodiment, an example is described in which individual difference-based corrections are performed. However, blood pressure measurements are not limited to only to individual-based corrections, and thus corrections based on other factors may be performed. As an example, a sticker-type pulse wave sensor according to the present variation has a built-in acceleration sensor.

The acceleration sensor included in the sticker-type pulse wave sensor according to present variation transmits a measurement result to the control device 1400. Also, the control device 1400 calculates a relative positional relationship between the position of the sticker-type pulse wave sensor and the heart based on the measurement result of the acceleration sensor and corrects the blood pressure based on the positional relationship. It is to be noted that the blood correction technique performed based on the relative positional relationship is not limited to known techniques, and thus any technique may be used.

It is conceivable that as the relative positional relationship of the sticker-type pulse wave sensor and the heart changes, an error occurs in the blood pressure measurement. To address this, the control device 1400 of the present variation calculates the relative positional relationship and performs blood pressure correction based on the positional relationship, and thus the accuracy of blood pressure estimations can be increased.

The technique involving the use of the acceleration sensor in the present variation is by no means a limitation and thus another sensor such as a level that detects angular deviations with the direction of gravity may be used to detect a relative position of an arm or the like to which the sticker-type pulse wave sensor is affixed. Also, it is conceivable to use an acceleration sensor of Micro Electro Mechanical Systems (MEMS) that are mass-produced and are low in cost due to smartphones.

Also, initial settings and the like regarding the relative positional relationship between the sticker-type pulse wave sensor and the heart may be set by the test subject via a mobile terminal that can communicate with the sticker-type pulse wave sensor.

As a setting technique, it is conceivable to, for example, instruct the sticker-type pulse wave sensor via the mobile terminal to set, as an initial state, a state in which the arm has been lowered vertically.

The technique for calculating the relative position of an arm or the like is described. For example, the sticker-type pulse wave sensor adds up the degree of acceleration received from the acceleration sensor with respect to each of the three axes of the acceleration sensor. By converting the sum totals of each of the three axes to movement distances of each of the three axes, the extent to which the part, to which the acceleration sensor is affixed, such as the arm, moves, can be calculated.

In other words, although the upper arm portion and the like of the test subject can move at various angles, the three-axes acceleration sensor is used in the present variation. By doing so, the body actions such as the raising and lower of an arm and body actions such as walking, standing, and the like can be differentiated. Therefore, the control device of the sticker-type pulse wave sensor according to the present variation can calculate the raising and lowering of the arm based on signals from the acceleration sensor and can detect a difference in height with heart to make corrections.

### Fourth Embodiment

An example is described in LEDs are arranged around the PD 114 of the sticker-type pulse wave sensor according to the aforementioned embodiments in four directions. However, such an arrangement example is by no means a limitation. As such, in the fourth embodiment, an example is described in which the PD 114 (light detector) and the LED 113 are arranged in one-to-one correspondence. The sticker-type pulse wave sensor according to the present embodiment is affixed such that a line segment connecting the PD 114 and the LED unit 113 is perpendicular with respect to the artery running direction of the test subject.

Incidentally, in a typical pulse wave measurement, the main purpose is to provide information such as the oxygen saturation level and the number of pulse waves, for example. With respect to this, when a blood pressure estimation is to be made from the pulse wave sensor, there is demand for extremely high-accuracy waveform measurement of a pulse wave on the order of 1 msec. If an error as high as 10 msec occurs with respect to the relative position characteristic peak of a pulse wave, an error of ±10 mmHg will, in turn, occur with respect to the blood pressure estimation. As such, there is demand for extremely high-accuracy waveform measurement of a pulse wave by sphygmomanometer using a pulse wave sensor.

The pulse wave propagates along an artery. When the PD (detecting device) and the LED (light emitting device) are arrayed in parallel to the traveling direction, the light propagation path also becomes parallel, and thus the position near the heart and the position far from the heart are both encompassed. A deviation between the time at which the pulse wave propagates to the portion close heart and the time at which the pulse wave propagates to the portion far from the heart occurs equal to the speed of propagation. The deviation is approximately a pulse wave propagation time of 1 msec with respect to a 10 mm length of artery, and when this deviation occurs, a blood pressure estimation error will be approximately several mmHg for high accuracy pulse wave time measurement.

In order to address this, in the sticker-type pulse wave sensor according to the present embodiment, the PD (detecting device) 114 and the LED (light emitting device) 113 are arrayed such that the propagation path is perpendicular to the traveling direction of the artery. In other words, since the light propagation path and the traveling direction of the artery are perpendicular to each other, the PD (detecting device) 114 and the LED (light emitting device) 113 are at substantially the same distance with respect to the heart, and thus error can be suppressed.

It is considered that the sticker-type pulse wave sensor according to the present embodiment is affixed to the upper arm, for example. The sticker-type pulse wave sensor can be fixed for one day or longer (may be even two to three days, for example) depending on the adhesive layer, as in the aforementioned embodiments. For the upper arm as well, the inner side is preferable because the travelling position of the artery is close and there are fewer muscle artifacts.

### Fifth Embodiment

For the sticker-type pulse wave sensor according to the aforementioned embodiments, a technique is described in which there is one PD and either a pulse wave or blood pressure is measured. However, measurement may be performed using multiple PDs. As such, in the fifth embodiment, an example is described in which multiple PDs are used.

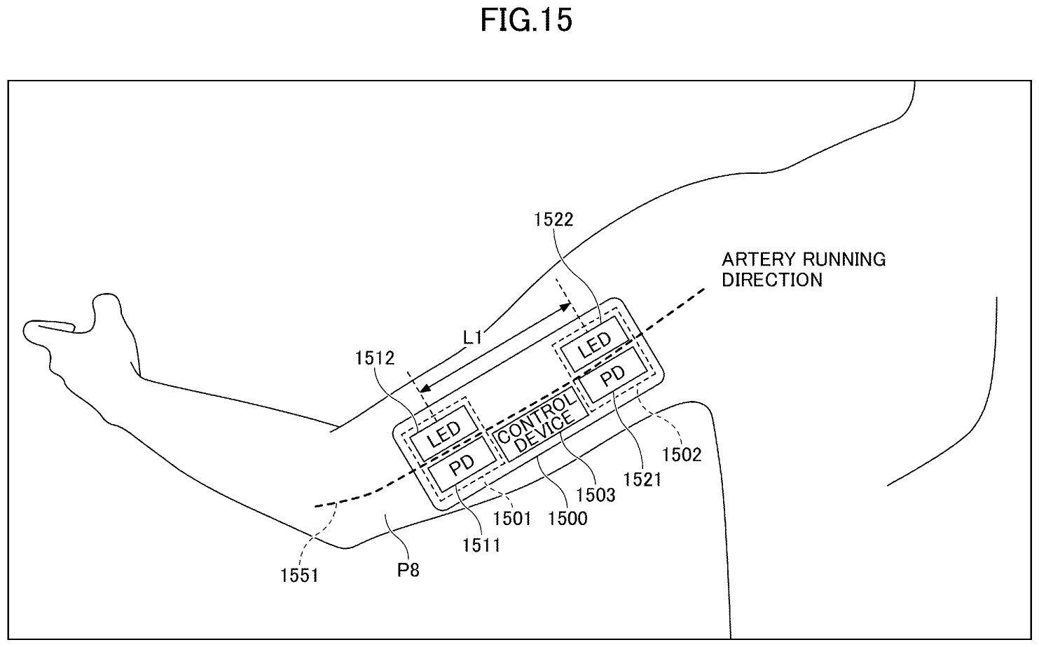

FIG. 15 is a conceptual diagram of a case in which a sticker-type pulse wave sensor 1500 according to the present embodiment (also functions as a sphygmomanometer) is affixed to an upper arm portion. The sticker-type pulse wave sensor 1500 illustrated in FIG. 15 is provided with two PD-LED units 1501 and 1502 in which a PD and an LED are provided in combination.

The PD-LED unit 1501 is a combination of a PD 1511 and an LED 1512, and the PD 1511 detects light output from the LED 1512. In the present embodiment as well, a shielding layer (not illustrated) is provided between the PD 1511 and the LED 1512 as is the case for those in the previously-described embodiments.

The PD-LED unit 1502 is a combination of a PD 1521 and an LED 1522, and the PD 1521 detects light output from the LED 1522. In the present embodiment as well, a shielding layer (not illustrated) is provided between the PD 1521 and the LED 1522 as is the case for those in the previously-described embodiments.

Similarly to that in the fourth embodiment, each of the PD-LED units 1501 and 1502 of the sticker-type pulse wave sensor 1500 are configured such that line segments connecting the PD and LED of the corresponding PD-LED units 1501 and 1502 are perpendicular to the artery running direction of test subject P8.

Furthermore, in the present embodiment, the PD-LED unit 1501 and the PD-LED unit 1502 are separated from each other by a predetermined distance L1. The distance L1 may be adjusted to suit the embodiment, and the distance L1 may be 10 cm, for example.

A control device 1503 measures a pulse wave and blood pressure based on signals from the PD-LED unit 1501 and 1502.

At such timing, the control device 1503 according to the present embodiment measures the pulse wave and the like by using the multiple PDs, that is, the PD 1511 and the PD 1521. The control device 1503 detects, from the multiple PDs 1511 and 1521, a common pulse wave having a phase shift corresponding to the time equivalent to the distance L1 between the PDs 1511 and 1521.

In other words, the control device 1503 calculates the pulse wave propagation time by taking into consideration the quantified phase shift and the feature of the pulse wave calculated from each of the PDs 1511 and 1521. In the present embodiment, the calculation of a more accurate pulse wave propagation time is achieved by taking into consideration the measurement results of the multiple PDs 1511 and 1521 and the distance between the PDs 1511 and 1521.

Furthermore, the control device 1503 performs a conversion into blood pressure based on the calculated pulse wave propagation time. By doing so, highly accurate blood pressure measurement can be achieved.

The present embodiment is by no means limited to the affixing of the sticker-type pulse wave sensor 1500 to the upper arm portion.

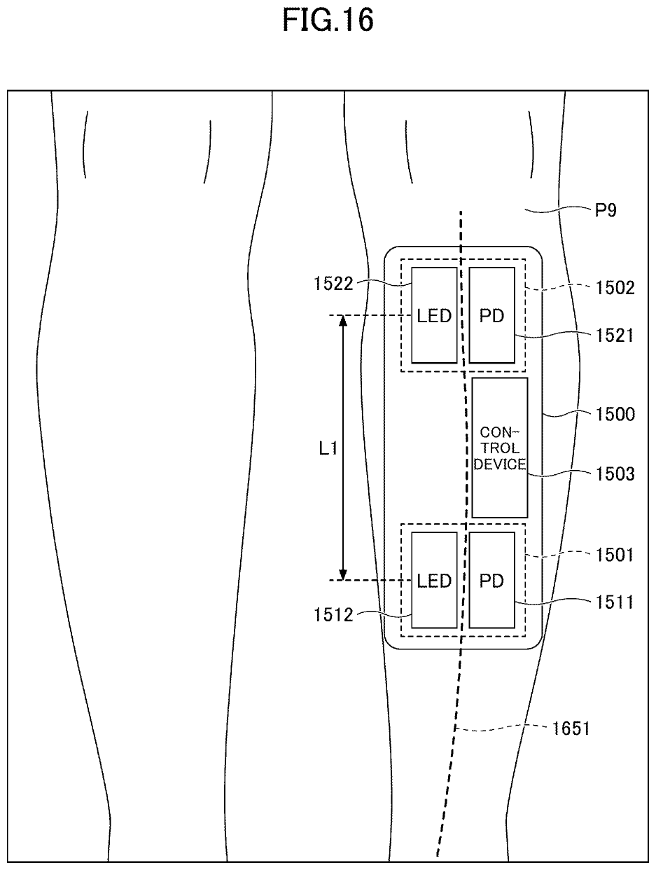

FIG. 16 is a conceptual diagram of a case in which the sticker-type pulse wave sensor 1500 according to the present embodiment is affixed to a leg. As illustrated in FIG. 16, the sticker-type pulse wave sensor 1500 may be affixed to any body portion as long as the sticker-type pulse wave sensor 1500 is affixed along the artery running direction.

### Sixth Embodiment

The multiple PD-LED units are not limited to a form in which the multiple PD-LED units are provided on a single flat component as is the case for the sticker-type pulse wave sensor 1500 according to the fifth embodiment. As such, in the sixth embodiment, a case in which two sticker-type pulse wave sensors are used is described.

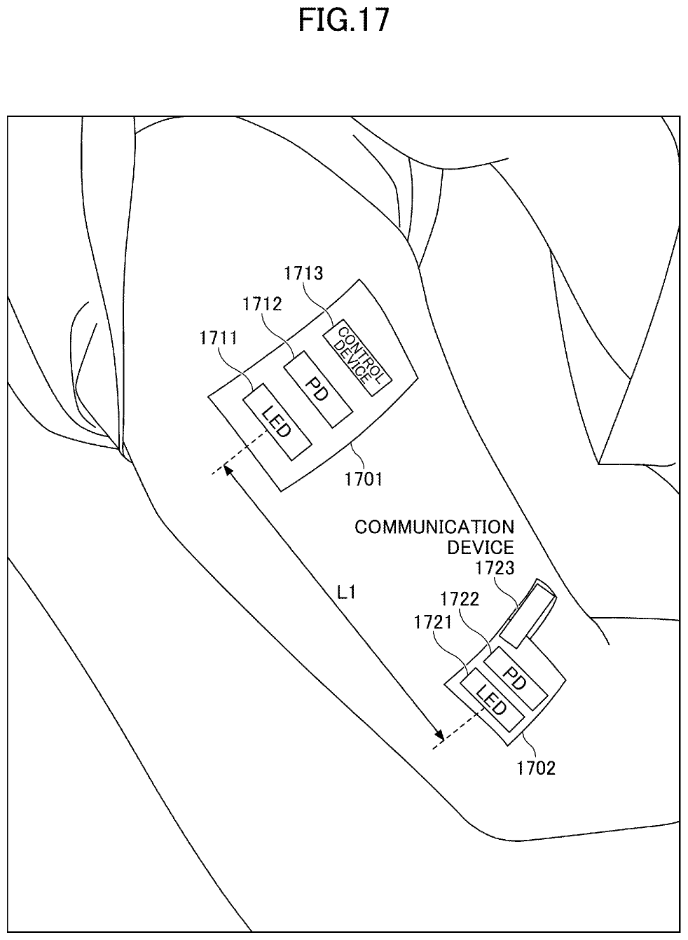

FIG. 17 is a conceptual diagram of a case in which multiple sticker-type pulse wave sensors 1701 and 1702 according to the present embodiment are affixed to an upper arm. FIG. 17 is an example in which the multiple sticker-type pulse wave sensors 1701 and 1702 are affixed along the artery running direction. Also, the distance L1 between the multiple sticker-type pulse wave sensors 1701 and 1702 is also set in advance to a distance as in the fifth embodiment.

In other words, in the present embodiment, the two sticker-type pulse wave sensors 1701 and 1702 are respectively affixed to the upper portion and to the lower portion of the upper arm. Since the distance between the two sticker-type pulse wave sensors 1701 and 1702 is set, a more accurate pulse wave propagation time can be calculated based on the phase shift between the two sticker-type pulse wave sensors 1701 and 1702.

The sticker-type pulse wave sensor 1702 includes an LED 1721, a PD 1722, and a communication device 1723. The PD 1722 detects light output from the LED 1721. In the present embodiment as well, a shielding layer (not illustrated) is provided between the LED 1721 and the PD 1722 as is the case for those in the previously-described embodiments. Also, the communication device 1723 transmits the detection result of the PD 1722 to a control device 1713.

The sticker-type pulse wave sensor 1701 includes an LED 1711, a PD 1712, and the control device 1713. The PD 1712 detects light output form the LED 1711. In the present embodiment as well, a shielding layer (not illustrated) is provided between the PD 1712 and the LED 1711 as is the case for those in the previously-described embodiment. Also, the control device 1713 performs a blood pressure measurement based on the detection result received from the communication device 1723 and a detection result of the PD 1712. The blood pressure measurement technique is omitted as the technique is the same as that of the fifth embodiment.

### Seventh Embodiment

In the aforementioned embodiments, a technique is described in which blood pressure is measured based on a pulse wave. However, the aforementioned embodiments are not limited to a technique using only a pulse wave when measuring blood pressure. Therefore, in the seventh embodiment, a case is described in which blood pressure is measured using a pulse wave and an electrocardiogram.

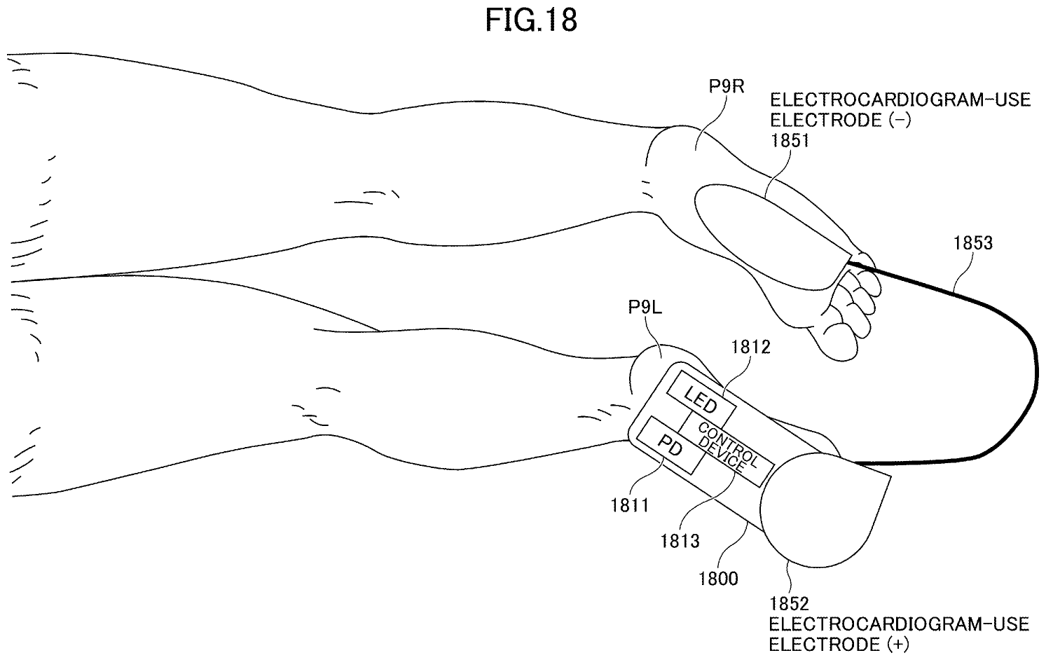

FIG. 18 is a conceptual diagram of a case in which a sticker-type pulse wave sensor 1800 according to the present embodiment and electrocardiogram-use electrodes 1851 and 1852 are affixed to soles of feet. FIG. 18 is an example in which the electrocardiogram-use electrode (−) 1851 is affixed to the sole of a right foot P9R and an electrocardiogram-use electrode (+) 1852 is affixed to the sole of a left foot P9L. The electrocardiogram-use electrode (−) 1851 and the electrocardiogram-use electrode (+) 1852 are connected by wiring 1853. Furthermore, the electrocardiogram-use electrode (+) 1852 is connected to the sticker-type pulse wave sensor 1800 so as to be capable of transmitting signals.

The sticker-type pulse wave sensor 1800 includes a PD 1811, an LED 1812, and a control device 1813. The PD 1811 detects light output from the LED 1812. In the present embodiment as well, a shielding layer (not illustrated) is provided between the PD 1811 and the LED 1812 as is the case for those in the previously-described embodiments.

Also, the control device 1813 of the sticker-type pulse wave sensor 1800 receives the detection results of the electrocardiogram-use electrode (−) 1851 and the electrocardiogram-use electrode (+) 1852. Then, the control device 1813 performs a blood pressure measurement based on the PD 1811 the detection results from electrocardiogram-use electrode (−) 1851 and the electrocardiogram-use electrode (+) 1852. Thus, the sticker-type pulse wave sensor 1800 also functions as a sphygmomanometer using a pulse wave and an electrocardiogram.

Incidentally, typical electrocardiogram involves a technique in which an electrical signal generated in conjunction with a muscle contraction of the heart. The electrodes of the electrocardiogram are detected by the potential difference between the positive and negative electrodes. Generally, the potential can be detected by attaching electrodes to the left and right of the heart.

Therefore, in the present embodiment, electrodes are fixedly placed on the right foot and the left foot. By fixedly placing the electrodes, the heart can be crossed as a current path, and thus the electrocardiogram (electrocardiogram waveform) can be detected with high accuracy.

Furthermore, in order to enhance the accuracy of a blood pressure measurement, it is necessary to accurately detect the pulse wave propagation time. The pulse wave propagation time is the time it takes for the pulse wave generated by a pulsation of the heart to propagate. Therefore, in the present embodiment, the pulse wave propagation time is calculated by combining the electrocardiogram for detecting the pulsation of the heart and the pulse wave.

Also, the sticker-type pulse wave sensor 1800 may be affixed to a heel of a foot, for example. Since peripheral nerves are concentrated in the foot (the heel, for example), the measurement accuracy can be enhanced.

For example, for a test subject in an intensive care unit or the like, the foot can be easily accessed because there are no other sensors fixedly placed there. Since such a test subject in unlikely to stand up much, the sticker-type pulse wave sensor 1800 can be provided on the sole of the foot or the like. For a test subject such as this who is often sleeping, the measurement can be easily performed because the location is easily accessible by a medical practitioner. Therefore, blood pressure can be easily measured continuously over a period of 24 hours.

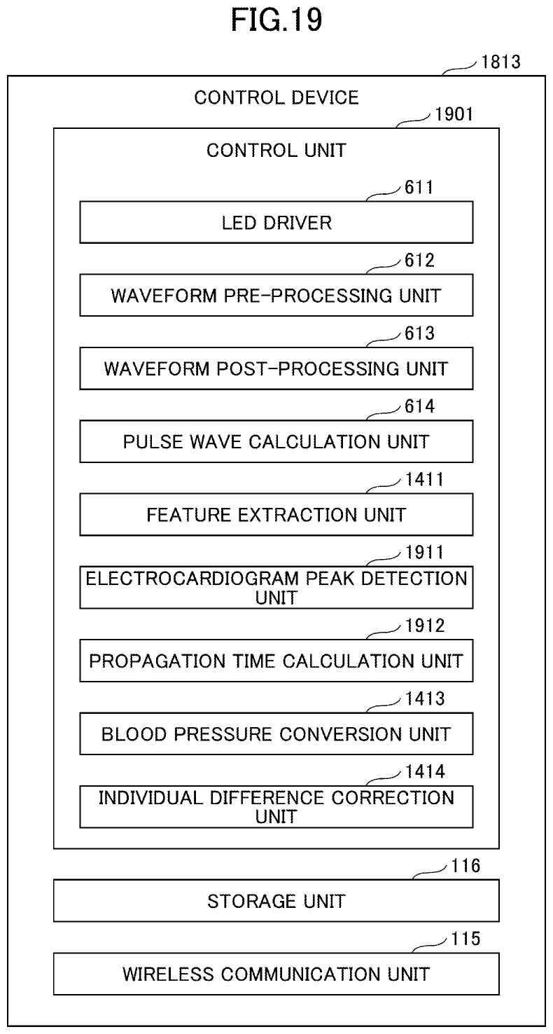

FIG. 19 is a block diagram illustrating a configuration to be executed by the control device 1813. The present embodiment is an example in which the control device 1813 is included instead of the control device 1400 according to the previously-described embodiments. Other components are substantially the same as those of the above-described embodiments and will not be described. The components that are the same as those of those of the previously-described embodiments are denoted by the same reference numerals and descriptions are omitted.

The control device 1813 is different from the control device 1400 according to the above-described embodiment in that the control device 1813 includes a control unit 1901 whose processing is different from that of the control unit 1401.

In the control unit 1901, compared to the control unit 1401, an electrocardiogram peak detection unit 1911 is added, and processing of a propagation time calculation unit 1912 is changed.

The electrocardiogram peak detection unit 1911 measures the electrocardiogram from the potential difference between the right foot and the left foot, which is the detection result of the electrocardiogram-use electrode (−) 1851 and the electrocardiogram-use electrode (+) 1852. Furthermore, the electrocardiogram peak detection unit 1911 detects, from the electrocardiogram, an R peak which is a pulsation of the heart.

The propagation time calculation unit 1412 calculates a pulse wave propagation time based on the feature of the pulse wave extracted by the feature extraction unit 1411 and the R peak detected by the electrocardiogram peak detection unit 1911. Specifically, the propagation time calculation unit 1412 calculates the pulse wave propagation time by measuring the time delay between the R peak and the rising position of the pulse wave. Subsequent processing is substantially the same as those in the previous-described embodiment and thus description is omitted.

In the present embodiment, by measuring biological information such as blood pressure in combination with an electrocardiogram, influences such as noise due to body movements can be reduced, and thus highly accurate blood pressure estimation can be achieved.

### Eighth Embodiment

In the above-described embodiments, application examples of the sticker-type pulse wave sensor were described. In contrast to these, in the present embodiment, a diagnostic service involving use of the sticker-type pulse wave sensor is described. The sticker-type pulse wave sensor also functions as a sphygmomanometer as described above. Therefore, this can also be referred as a diagnostic service involving use of a sphygmomanometer.

Conventionally, when 24-hour continuous measurement is to be performed, a cuff-type sphygmomanometer is fixedly placed by hand by a nurse, and therefore, a visit to the home of the patient by the nurse or an outpatient visit is necessary. Moreover, the measurement-based diagnosis cannot be carried out until the 24-hours continuous measurement is performed, the patient revisits the doctor, and the doctor reviews the measurement results. If the cuff comes off or an measurement error occurs during measurement, it is difficult to make corrections midway through the procedure.

By performing the steps described below in the diagnostic procedure according to the present embodiment, more a simplified long-term measurement can be achieved.

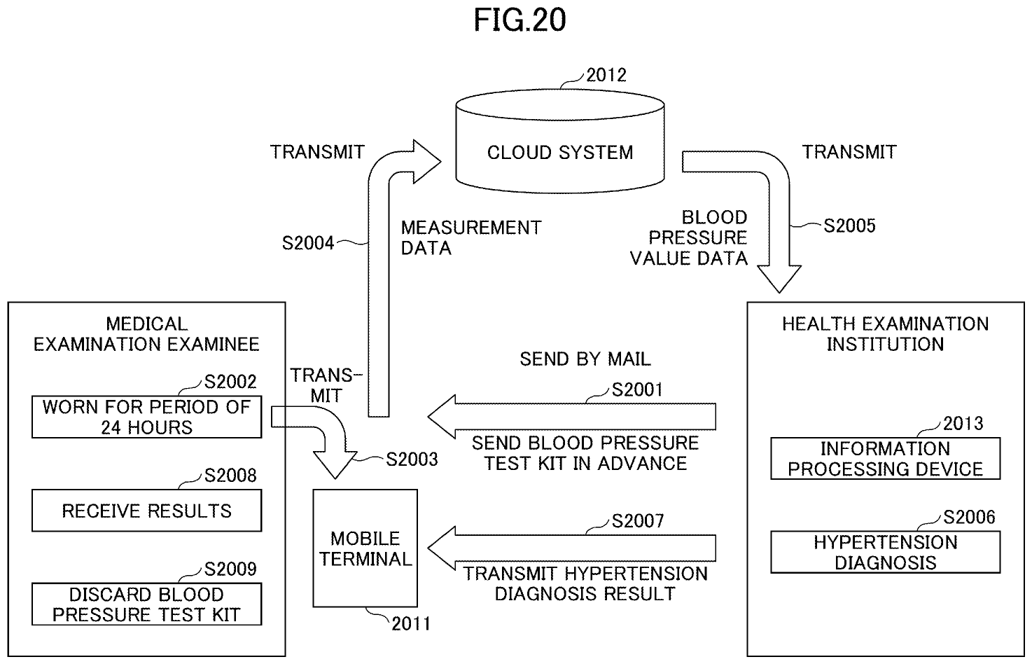

FIG. 20 is a diagram illustrating an example of a procedure of a diagnostic service according to the present embodiment. A person undergoing diagnosis (test subject) as illustrated in FIG. 20 has a mobile terminal 2011. A cloud system 2012 manages information of the diagnostic service. A health examination institution is provided with an information processing device 2013 for displaying information of the cloud system 2012. Next, the procedure is described in detail.

A health examination institution sends a blood pressure examination kit including a sticker-type pulse wave sensor in advance to the person who is to undergo a medical examination, i.e., the medical examination examinee. (S2001).

The medical examination examinee wears the received sticker-type pulse wave sensor (which also functions as a sphygmomanometer) for 24 hours by affixing it the part of the body to be diagnosed (S2002) At such timing, the medical examination examinee sets the sticker-type pulse wave sensor and the mobile terminal 2011 to be able to communicate.

The sticker-type pulse wave sensor (an example of the first transmitting unit) transmits the blood pressure estimation result and the detection result (an example of information determined based on the pulse wave of the test subject) of the PD 2112 to the mobile terminal 2011 (an example of the first communication device) (S2003: an example of the first transmitting step). Then, the mobile terminal 2011 (an example of the second transmitting unit) transmits measurement data (including blood pressure estimation results and PD 2112 detection results) indicating the measurement results of the information regarding blood pressure to the cloud system 2012 (an example of the second communication apparatus) via the public network (S2004: an example of the second transmission step).

The cloud system 2012 manages measurement data indicating the estimation results of the blood pressure and the detection results of the PD 2112. The cloud system 2012 regularly confirms malfunction of the sticker-type pulse wave sensor and erroneous detection of the sticker-type pulse wave sensor based on the received measurement data, and estimates the blood pressure value of the medical examination examinee based on the received measurement data. Since the estimation takes into account various parameters, highly-accurate estimation, estimation can be performed with higher accuracy than with the estimation results of the sticker-type pulse wave sensor.

The cloud system 2012 transmits information regarding the estimated blood pressure value to the information processing device 2013 of the health examination institution (S2005). The information processing device 2013 (an example of the display unit) of the health examination institution displays information regarding the estimated blood pressure value, and the physician makes a diagnosis of the medical examination examinee, for example, a diagnosis of hypertension, based on the information regarding the estimated blood pressure value (S2006: an example of the display step).

The information processing device 2013 (an example of the third transmitting unit) transmits the diagnostic result of hypertension or the like to the mobile terminal 2011 of the medical examination examinee (S2007: an example of the third transmission step) by operation performed by the physician.

The medical examination examinee confirms the diagnostic result of hypertension or the like on the mobile terminal 2011 (S2008). After doing so, the medical examination examinee discards the blood pressure test kid including the sticker-type pulse wave sensor (S2009).

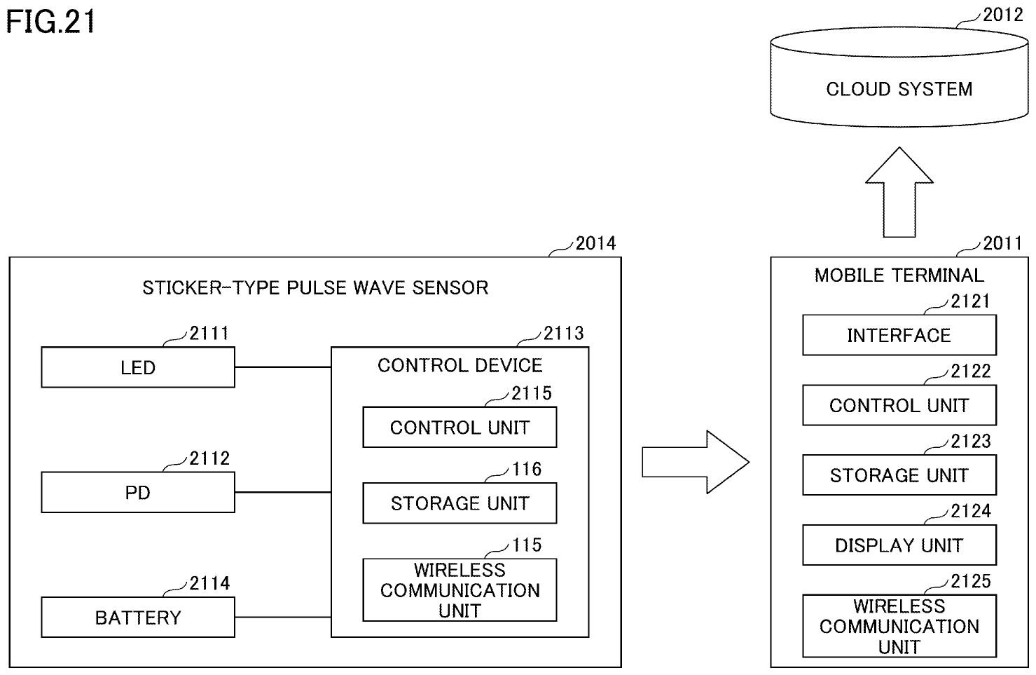

FIG. 21 is a diagram illustrating an example of a diagnostic system to be used with the diagnostic service according to the present embodiment. As shown in FIG. 21, the diagnostic system includes at least the mobile terminal 2011, the cloud system 2012, and the sticker-type pulse wave sensor 2014.

The sticker-type pulse wave sensor 2014 includes an LED 2111, the PD 2112, a control device 2113, and a battery 2114. The sticker-type pulse wave sensor 2014 is driven by power supplied from the battery 2114, and the PD 2112 detects light reflected by a body part of the medical examination examinee among the light emitted from the LED 2111. The sticker-type pulse wave sensor 2014 according to the present embodiment also includes a shielding layer as in the previously-described embodiments. The sticker-type pulse wave sensor 2014 is driven by power supplied from the battery 2114 and the PD 2112 detects light reflected by a body part of the medical examination examinee among the light emitted from the LED 2111. The control unit 2015 of the control device 2113 measures blood pressure or the like from the detection result of the PD 2112, and transmits the measurement result, the detection result of the PD 2112, or the like to the mobile terminal 2011 by using a wireless communication unit 115. The blood pressure estimation technique is substantially the same as same omitted as in the above-described embodiment.

The mobile terminal 2011 includes an interface 2121, a control unit 2122, storage unit 2123, a display unit 2124, and a wireless communication unit 2125. A program that performs the processing below is executed in the control unit 2122.

The wireless communication unit 2125 stores the detection result or the like received from the sticker-type pulse wave sensor 2014 into the storage unit 2123 and transmits the detection result or the like to the cloud system 2012.

Further, in the mobile terminal 2011, the display unit 2124 displays the result of blood pressure estimation, which is estimated based on the detection result, in accordance with the operation received by the interface 2121 from the medical examination examinee.

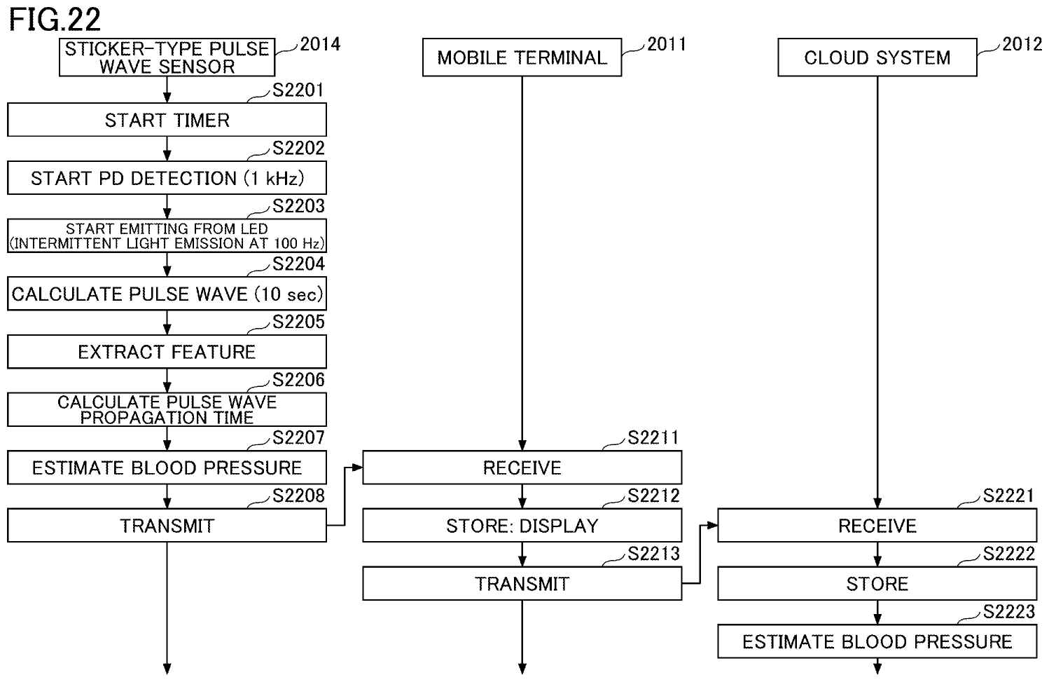

FIG. 22 is a sequence diagram illustrating processing to be performed by the diagnostic system according to the present embodiment.

The sticker-type pulse wave sensor 2014 initiates the start of the timer after being affixed to a body part of the medical examination examinee (S2201).

The PD 2112 of the sticker-type pulse wave sensor 2014 starts detection (S2202). The detection interval of the PD 2112 is 1 kHz, for example.

The LED 2111 of the sticker-type pulse wave sensor 2014 starts detection (S2203). The light emission interval of the LED 2111 is 100 Hz, for example.

The pulse wave calculation unit 614 calculates a pulse wave (an example of information related to the pulse wave) based on the detection information that is the detection result of the PD 2112 (S2204).

The feature extraction unit 1411 extracts a feature (for example, peaks of the PW and the TW, and so on) (S2205)

The propagation time calculation unit 1412 calculates the pulse wave propagation time based on the feature of the pulse wave (S2206).

The blood pressure conversion unit 1413 estimates the blood pressure based on the calculated pulse wave propagation time (S2207). At such timing, a correction may be made based on individual differences or the like.

The wireless communication unit 115 transmits both the estimation result of the blood pressure estimated based on the pulse wave and the detection result of the PD 2112 (an example of information obtained based on the pulse wave of the test subject) to the mobile terminal 2011 (an example of the first communication device) (S2208).

A wireless communication unit 2125 of the mobile terminal 2011 receives the estimation result of the blood pressure and the detection result of the PD 2112 (S2211).

The control unit 2122 of the mobile terminal 2011 stores the received blood pressure estimation result and the detection result of the PD 2112 into the storage unit 2123, and displays the blood pressure estimation result on the display unit 2124 in response to an operation from the user (S2212).

The wireless communication unit 2125 of the mobile terminal 2011 transmits the blood pressure estimation result and the detection result of the PD 2112 to the cloud system 2012 (an example of the second communication apparatus) via a public network (S2213).

The cloud system 2012 receives the estimation of the blood pressure and the detection result of the PD 2112 from the mobile terminal 2011 (S2221).

The cloud system 2012 accumulates information indicating the estimation result of the blood pressure and the detection result of the PD 2112 (S2222).

The cloud system 2012 estimates blood pressure based on the accumulated information (S2223). Since various parameters are stored in the cloud system 2012, blood pressure estimation can be performed with higher accuracy than with the sticker-type pulse wave sensor 2014.

In the diagnosis system according to the present embodiment, since the above-described configuration is provided, the diagnostic result of the physician can be obtained easily without any need for the medical examination examinee to go to the hospital or for a medical practitioner to visit the home of the medical examination examinee.

In the diagnostic system according to the present embodiment, a determination can be made as to whether or not the cloud system 2012 is operating normally based on information received in real-time from the sticker-type pulse wave sensor 2014. Furthermore, since a third party such as a medical practitioner can confirm the measurement result, a blood pressure estimation error due to malfunction can be suppressed.

In recent years, the cost of general purpose semiconductors has been decreasing owing to the advancement of IoT. Therefore, even when the sticker-type pulse wave sensor is used only once and disposed thereafter, this sticker-type pulse wave sensor can be realized at a low cost. Disposability provides various advantages, such as eliminating the need for reuse of the adhesive layer, achieving a simple waterproof package that does not require any charging, saving one the trouble of having to send the sensor back by mail, and reducing sanitary considerations.

In the sticker-type pulse wave sensor, the blood pressure can be estimated based on the waveform of the detected pulse wave, and the change in the blood pressure value can be recorded in the cloud system 2012 or the like. For example, variations in blood pressure during the day and night are important information, and in this embodiment, such information can be accumulated in the cloud system 2012 with the above-described configuration. Furthermore, a medical practitioner can confirm such information. This makes it easier to determine subtypes of hypertension such as masked hypertension and white-coat hypertension. Furthermore, the medical examination examinee can easily receive the determination result.

### Ninth Embodiment

The configuration of the sticker-type pulse wave sensor is not limited to the above-described embodiments, and various aspects can be considered. Therefore, in the ninth embodiment, another aspect of the sticker-type pulse wave sensor is described together with processing involving use of the sticker-type pulse wave sensor.

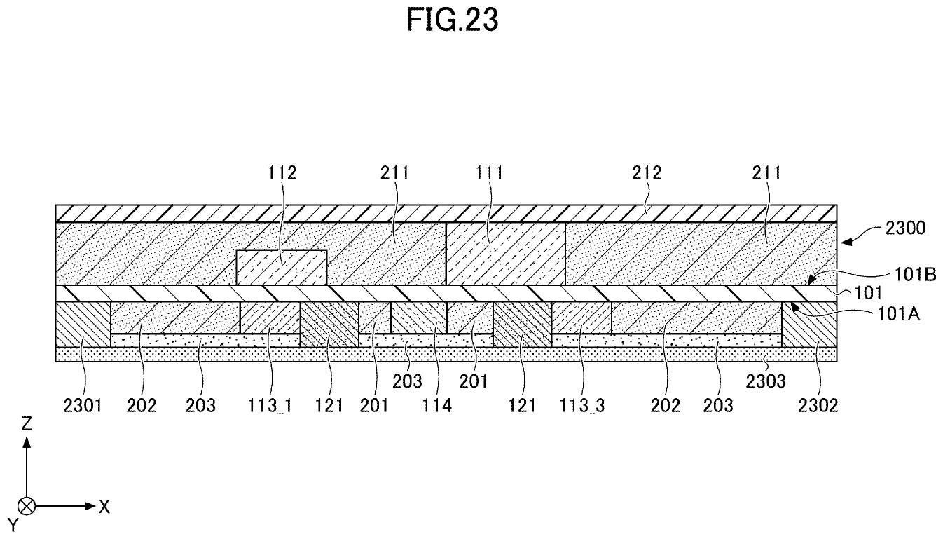

FIG. 23 is a cross-sectional view illustrating an optical system of a sticker-type pulse wave sensor 2300 according to the present embodiment. The components that are the same as those of the aforementioned sticker-type pulse wave sensor 100 are denoted by the same reference numbers and descriptions are omitted.

The sticker-type pulse wave sensor 2300 according to the present embodiment further includes a first electrode 2301 and a second electrode 2302 compared to the sticker-type pulse wave sensor 100. Before the sticker-type pulse wave sensor 2300 is fixedly placed on the test subject, a protective sheet 2303 affixed to the adhesive layer 203.

The protective sheet 2303 is manufactured such that the protective sheet 2303 contains, for example, carbon black at a predetermined ratio or more. Therefore, the protective sheet 2303 has conductivity.

In other words, the electrical conduction between the first electrode 2301 and the second electrode 2302 changes depending on the presence or absence of the protective sheet 2303. A signal indicating a change in electrical conduction between the first electrode 2301 and the second electrode 2302 is output to the control device 112. Thus, the control device 112 can recognize that the protective sheet 2303 has been peeled off. Upon confirming that the protective sheet 2303 has been peeled off, the control device 112 can start measuring the biological information about the test subject.

A sensor ID for identifying the sticker-type pulse wave sensor 2300 may be stored in the storage unit 116 of the control device 112. The sensor ID may be identification information for a disposable.

The sticker-type pulse wave sensor 2300 according to the present embodiment is used, for example, when the diagnostic system according to the present embodiment is to determine a subtype of hypertension such as masked hypertension or white-coat hypertension.

For example, the sticker-type pulse wave sensor 2300 may be a disposable measuring device as those in above-described embodiments. Therefore, the sticker-type pulse wave sensor 2300 may be delivered to the home of the test subject. Thus, the sticker-type pulse wave sensor 2300 can enable easy measurement at the home of the test subject. Moreover, the diagnostic system according to the present embodiment can obtain the determination of the medical institution based on the measurement result of the sticker-type pulse wave sensor 2300.

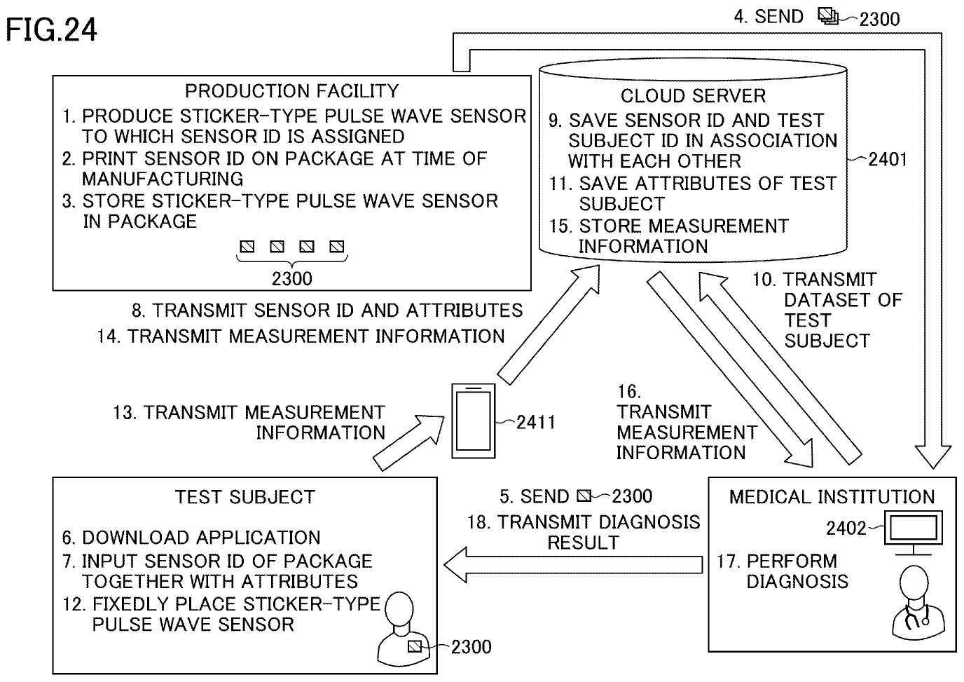

With the diagnostic service according to the present embodiment, a diagnosis can be made for each test subject. FIG. 24 is a diagram illustrating a diagnostic system to be used with the diagnostic service according to the present embodiment. In the diagnostic system according to the present embodiment, a (disposable) sensor ID uniquely allocated to each disposable sticker-type pulse wave sensors 2300 described above is used.

As shown in FIG. 24, the diagnostic system includes a production facility, a medical institution, a test subject, and a cloud server 2401.

The production facility according to the present embodiment produces a sticker-type pulse wave sensor 2300 to which a sensor ID is assigned. The sensor ID is registered, for example, in the storage unit 116 in the control device 112 of the sticker-type pulse wave sensor 2300.

At the production facility the sensor ID is printed on the package when the sticker-type pulse wave sensor 2300 is manufactured. At the production facility, the sticker-type pulse wave sensor 2300 is stored in a package. In the present embodiment, one sticker-type pulse wave sensor 2300 is stored in one package. The sensor ID printed on the package is a sensor ID for identifying the stored sticker-type pulse wave sensor 2300. The production facility sends the sticker-type pulse wave sensor 2300 stored in the package to the medical institution.

The medical institution is an institution for diagnosing test subjects and includes medical personnel and a terminal 2402 used by the medical personnel.

The medical institution sends the sticker-type pulse wave sensor 2300, which is sent from the production institution, to the test subject to be diagnosed. In the present embodiment, an example in which the medical institution sends the sticker-type pulse wave sensor 2300 to the test subject is described, but the method of sending the sticker-type pulse wave sensor 2300 to the test subject is by no means limited, and for example, the production facility may send the sticker-type pulse wave sensor 2300 directly to the test subject or may send the sticker-type pulse wave sensor 2300 to the test subject by way of another institution such as a pharmacist.

The test subject carries a communication terminal 2411. The communication terminal 2411 has the same configuration as the mobile terminal 2011 described above. The communication terminal 2411 can communicate with various communication devices (a cloud server 2401, for example) connected to a public network (not illustrated).

The cloud server 2401 manages information regarding the test subject necessary for the diagnosis involving use of the sticker-type pulse wave sensor 2300. In the present embodiment, the cloud server 2401 is composed of one or a plurality of communication devices (including the information processing device) terminals, and can provide various services.

The test subject uses the communication terminal 2411 to access the address provided together with the sticker-type pulse wave sensor 2300 by the medical institution. By doing so, the communication terminal 2411 downloads and installs an application for performing the measurement. Thereafter, the communication terminal 2411 executes the application to display an input screen for inputting attributes of the test subject.

The communication terminal 2411 receives the input of the sensor ID indicated on the package and information for identifying the test subject (for example, the test subject ID) via the input screen. The communication terminal 2411 transmits the received the sensor ID and the test subject ID, which were input, to the cloud server 2401.

By doing so, the cloud server 2401 registers the sensor ID and the test subject ID in association with each other. Table 1 illustrates the correspondence relationship between the sensor ID and the test subject ID registered in the cloud server 2401.

TABLE 1

| | | Sicker-type pulse |
|---|---|---|
| | Test subject ID | wave sensor ID |
| 1 | A000001 | S000001 |
| 2 | A000002 | S000002 |
| 3 | A000003 | S000003 |

Further, the communication terminal 2411 may receive the input of the attribute of the test subject through the input screen. The attributes for which inputs are received include, for example, the name of the test subject, the address of the test subject, and the telephone number (an example of identification information of the communication terminal) of the communication terminal (a smartphone, for example) used by the test subject. The attribute is by no means limited to such information, and may include various types of information such as a password, age, gender, weight, presence or absence of any underlying disease, details of the underlying disease, physical condition, body temperature, and the like. Also, the communication terminal 2411 associates the sensor ID, the test subject ID, and the attribute with one another, and transmits them to the cloud server 2401. By doing so, the cloud server 2401 can manage the test subject ID and the attribute of the test subject in association with each other.

In the present embodiment, the preparation for measurement involving use of the sticker-type pulse wave sensor 2300 of the test subject is performed by executing the processing corresponding to the above-described inputs. The measurement of the test subject is, for example, a 24-hour measurement of the blood pressure or the like.

The medical institution may also transmit information (a dataset) regarding the test subject together with the test subject ID to the cloud server 2401. By doing so, the cloud server 2401 can manage information regarding the test subject in association with the test subject ID. The information (dataset) regarding the test subject includes, for example, other measurement data such as a complete medical checkup held by the medical institution. Hence, a detailed diagnosis can be realized.

The test subject fixedly places the sticker-type pulse wave sensor 2300 on an upper arm portion, a clavicle portion, or the like in accordance with the guidance of the installed application.

At such timing, the test subject peels off the protective sheet 2303 from the sticker-type pulse wave sensor 2300. The control device 112 of the sticker-type pulse wave sensor 2300 can recognize that the protective sheet 2303 has been peeled off based on a signal indicating a change in electrical conduction between the first electrode 2301 and the second electrode 2302. Upon doing so, the control device 112 starts the control for performing measurement.

The communication terminal 2411 and the sticker-type pulse wave sensor 2300 are connected by wireless communication. Thus, the application of the communication terminal 2411 confirms that the sensor ID for which an input was received matches with the sensor ID included in the information transmitted from the sticker-type pulse wave sensor 2300. Then, application of the communication terminal 2411 transmits the confirmation result to the cloud server 2401. By doing so, the cloud server 2401 can recognize whether or not the associated information is appropriate.

Thereafter, the sticker-type pulse wave sensor 2300 performs starts measurement and then transmits information (hereinafter, referred to as measurement information) indicating the measurement result to the communication terminal 2411. Then, the communication terminal 2411 transmits the sensor ID together with the measurement information to the cloud server 2401. The cloud server 2401 measurement information stores the measurement information, which is received together with the sensor ID, in association with the test subject ID corresponding to the sensor ID.

The terminal 2402 of the medical institution acquires and displays the information of the test subject and the information of the test subject and measurement information from the cloud server 2401. The terminal 2402 of a medical institution receives an input of a diagnostic result of the test subject from a physician. Thereafter, the terminal 2402 of the medical institution transmits the diagnostic result to the communication terminal 2411 of the test subject. The measurement information used in the diagnosis includes, for example, a change in the blood pressure of the test subject over a period of 24 hours, but the diagnosis target is by no means limited to blood pressure, and as such, other information may be used.

In the present embodiment, although an example is described in which the communication terminal 2411 transmits the sensor ID and the test subject ID, the transmission of the sensor ID and the test subject ID is by no means limited to the communication terminal 2411. For example, the terminal 2402 of the medical institution may transmit, to the cloud server 2401, the sensor ID of the sticker-type pulse wave sensor 2300 and the information (including the test subject ID) of the test subject to whom the sticker-type pulse wave sensor 2300 is sent.

By managing the sensor ID of the sticker-type pulse wave sensor 2300, the cloud server 2401 can reissue the same sensor ID. Although the disposable sticker-type pulse wave sensor requires a ID countless times, because the ID is reused, there is no lack of IDs.

### <Model-Based Corrections>

In the present embodiment, the medical institution is by no means limited to a method for diagnosing the test subject by referring to the measurement information (for example, a change in blood pressure over a period of 24 hours) received from the cloud server 2401.

In other words, the cloud server 2401 according to the present embodiment may correct the measurement information to facilitate diagnosis prior to transmission to the terminal 2402 of the medical institution. In the present embodiment, the cloud server 2401 stores the information of the test subject illustrated in Table 2, for example. The information of the test subject illustrated in Table 2 is stored in association with the test subject ID (the test subject ID: A000001, for example). In order for the cloud server 2401 to accumulate the test subject information (e.g., age, gender, weight, height, blood test value (cholesterol levels), blood glucose level, and any underlying disease (by selection)) illustrated in Table 2, for example, the communication terminal 2411 may receive the input of the information on the input screen, for example. The information of the test subject to be used for correction is not limited to the information input by the test subject, and as such, may be, for example, the results of a blood test or the like performed at the same or another medical institution after obtaining the consent of the test subject.

TABLE 2

| Number | Item | Value | |
|---|---|---|---|
| 1 | Age | 49 | |
| 2 | Gender | Male | |
| 3 | Weight | 62 | kg |
| 4 | Height | 172 | cm |

| | | |
|---|---|---|
| 5 | Cholesterol level | 120 |
| 6 | Blood glucose level | 110 |
| 7 | Underlying disease | None |

Further, the cloud server 2401 extracts the feature of the test subject from the information regarding the test subject, and models the test subject based on the extracted feature, and models the test subject. After doing so, the cloud server 2401 corrects the measurement information based on the model of the test subject. Then, the cloud server 2401 transmits the corrected measurement information to the terminal 2402 of the medical institution. By performing this processing, the medical institution can make a diagnosis in consideration of various factors such as age and body shape, so that a highly accurate diagnosis can be realized.

### Tenth Embodiment

In the aforementioned embodiments, an example is described in which a determined is made based on the measurement result, such as blood pressure or the like of the test subject. However, when diagnosing a test subject, it is preferable perform the diagnosis in consideration of an event actually being performed by the test subject. Therefore, in the diagnostic system according to the tenth embodiment, a case where an event being performed by the test subject is input is described.

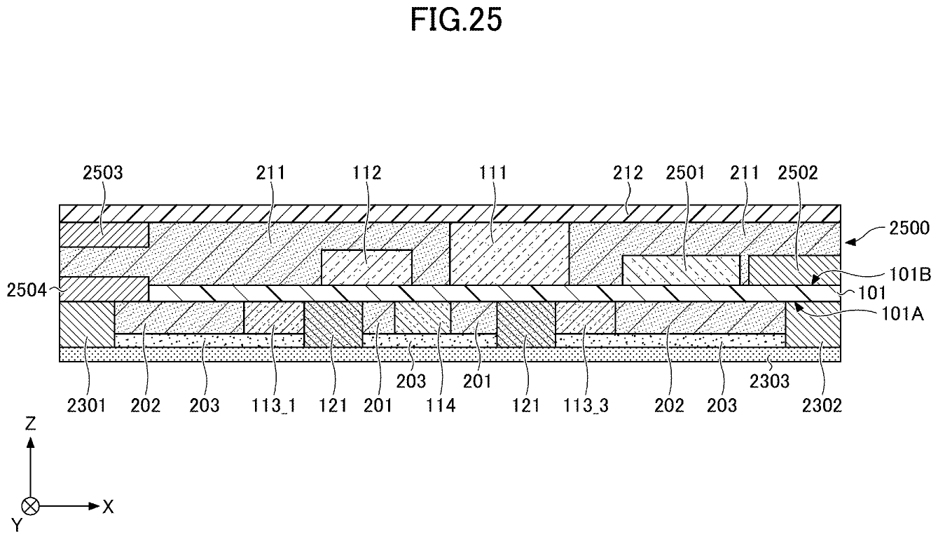

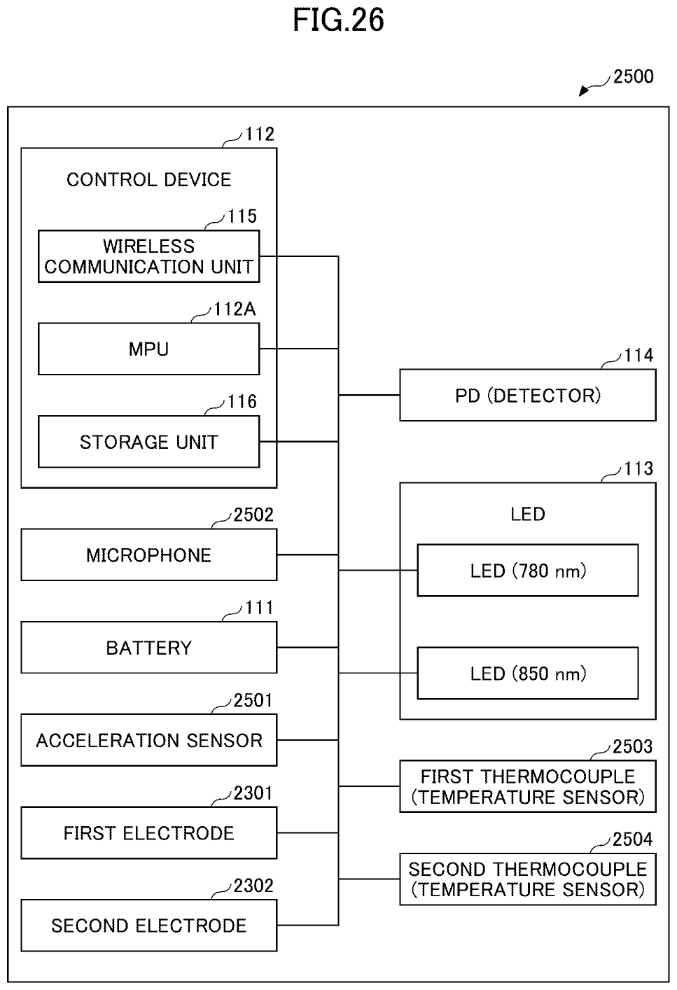

FIG. 25 is a cross-sectional view illustrating an optical system of a sticker-type pulse wave sensor 2500 according to the present embodiment. FIG. 26 is a diagram illustrating a block configuration of a sticker-type pulse wave sensor according to the present embodiment. The same reference numerals are assigned to the same configuration as that of the above-described sticker-type pulse wave sensor 2300, and description thereof will be omitted. The components of that are the same as those of the aforementioned sticker-type pulse wave sensor 2300 are denoted by the same reference numbers and descriptions are omitted.

The control device 112 according to the present embodiment may include an MPU 112A as a configuration for executing a program. The MPU 112A can measure the test subject by executing the program stored in the storage unit 116.

As illustrated in FIG. 26, the LED 113 may include an LED that outputs a wavelength of 780 nm and an LED that outputs a wavelength of 850 nm. With the sticker-type pulse wave sensor 2500 being capable of outputting two types of wavelengths, the sticker-type pulse wave sensor 2500 can calculate the oxygen saturation level of hemoglobin.

As illustrated in FIG. 25, the sticker-type pulse wave sensor 2500 according to the present embodiment further includes an acceleration sensor 2501, a microphone 2502, a first thermocouple 2503, and a second thermocouple 2504 compared to the sticker-type pulse wave sensor 2300.

The acceleration sensor 2501 detects acceleration of the test subject and outputs a detection result to a control device 112. The microphone 2502 detects sound around the test subject (including conversation and the like with the test subject) and outputs the detection result to the control device 112.

The control device 112 detects the body temperature of the test subject and the ambient air temperature (or indoor temperature) of the test subject based on signals input from the first thermocouple 2503 and the second thermocouple 2504. Although the present embodiment describes an example of detecting the body temperature of the test subject and the ambient temperature (or indoor temperature) of the test subject, one or more of the body temperature of the test subject and the ambient temperature (or room temperature) of the test subject may be detected.

### <Event Detection Function>

The control device 112 determines what state (hereinafter, referred to as event information) the test subject is in based on the acceleration sensor 2501, the microphone 2502, and the first and second thermocouples 2503 and 2504. The event information may be, for example, an item illustrated in Table 3 below.

TABLE 3

| | |
|---|---|
| 1 | Getting up after sleeping |
| 2 | Going to bed |
| 3 | Going to the bathroom |
| 4 | Bathing |
| 5 | Standing |
| 6 | Sitting |
| 7 | Being administered medicine |
| 8 | Eating |
| 9 | Drinking alcohol |
| 10 | Exercising |

In this manner, the control device 112 acquires, based on the detection result of the above-described sensor, an event number (an example of the event information) indicating whether the test subject is in a state of getting up after sleeping, going to bed, going to the bathroom, bathing, standing, sitting, being administered medicine, eating, drinking alcohol, or exercising. Table 3 illustrates an example of an event number and other events may be included. The event number is preferably information in which items that affect blood pressure are divided into categories.

Then, the control device 112 transmits the acquired event number in association with the acquired time to the communication terminal 2411.

The communication terminal 2411 can display the event number in association with blood pressure as a time series. Then, the test subject can confirm whether or not the event number displayed on the communication terminal 2411 is consistent with the actual action. If it is determined that even number is not consistent with the actual action, the test subject may perform an operation to correct the event number with respect to the communication terminal 2411.

The communication terminal 2411 transmits the time at which measurement was performed, the event number, and the measured information (including blood pressure) in association with the sensor ID in association with the measurement information (including blood pressure) to the cloud server 2401.

The cloud server 2401 receives the measurement information in time-series order. The measurement information includes information for identifying the blood pressure. In other words, the cloud server 2401 receives changes (waveform, for example) in the blood pressure representing the blood pressure of the test subject in time-series order, as acquired by the sticker-type pulse wave sensor 2500.

The cloud server 2401 receives the measurement information together with an event number indicating the event that occurred with the test subject in the time-series order.

The cloud server 2401 stores the information (measurement information and event numbers) received in time-series order in association with the test subject ID.

The acquisition of event information is not limited to the technique performed by the control device 112 of the sticker-type pulse wave sensor 2500. For example, the communication terminal 2411 of the test subject may identify the event number (example of event information) based on the information input from the sticker-type pulse wave sensor 2500. The event number may be acquired by any technique, for example, by using an AI-trained learning model trained by the events and the detection results of the sensor.

### <Event Inputting>

The present embodiment is not limited to an example in which the sticker-type pulse wave sensor 2500 acquires an event number (an example of event information). For example, the communication terminal 2411 of the test subject may have an event input function.

The communication terminal 2411 can receive inputs of event information performed by the test subject, for example. Event numbers that can be input include those that are of value to the physician's diagnosis, such as, for example, taking medicine, eating, going to the bathroom, bathing, and so on. For example, the communication terminal 2411 can receive a selection of an event number from a display screen of an application. When the input of the event number is received, a time stamp is issued and the communication terminal 2411 transmits, to the cloud server 2401, the event number in association with vital measurement information including blood pressure.

Thus, the cloud server 2401 can receive and store the measurement information and the event information in time-series order. The medical institution can evaluate drug efficacy or the like based on the information stored in the cloud server 2401.

Selectable event information may include, for example, taking medicine, eating (bread, rice, one slice of bread, two slices of bread, one serving of rice, two servings of rice, . . . ), drinking alcohol (type and amount), going to the bathroom, sleeping, and exercising (running and cycling). Further, the communication terminal 2411 may be configured such that detailed information to be conveyed to a physician, such as the contents of meals, body temperature at time of temperature check, physical condition, or mood, can be input by voice, test, or the like.

The cloud server 2401 generates, based on the accumulated information and in time-series order, a graph in which changes in blood pressure (changes in measurement information) of the test subject and events in the time-series order as they occurred during measurement of the test subject are illustrated in superimposed form. Then, the cloud server 2401 transmits, to the terminal of the medical institution, a graph in which the changes in blood pressure and the events in time-series order are superimposed onto each other.

The terminal 2402 of the medical institution displays a graph in which the blood pressure changes and the events are superimposed onto each other. The graph in which the changes in the blood pressure of the test subject and the events are superimposed onto each other in time-series order is not limited to the technique generated by the cloud server 2401, and as such, may be generated by the terminal 2402 of the medical institution. For example, the terminal 2402 of the medical institution includes an MPU as a configuration for executing a program, and the MPU executes the program stored in a storage unit (not illustrated) to generate a graph or display information necessary for diagnosis.

Thus, for example, the terminal 2402 of the medical institution can display the blood pressure of the test subject in time series based on the measurement information acquired from the sticker-type pulse wave sensor 2500.

If in the graph displayed on the terminal 2402 of the medical institution, for example, the timing of bathing coincides with the sudden rise in blood pressure and, the medical personnel belonging to the medical institution may diagnose that the sudden rise in blood pressure is not problematic. In this way, by displaying the measurement information in association with the event, the noise component displayed on the graph can be disregarded upon visual inspection, and thus the diagnostic accuracy can be improved.

### <Diagnosis>

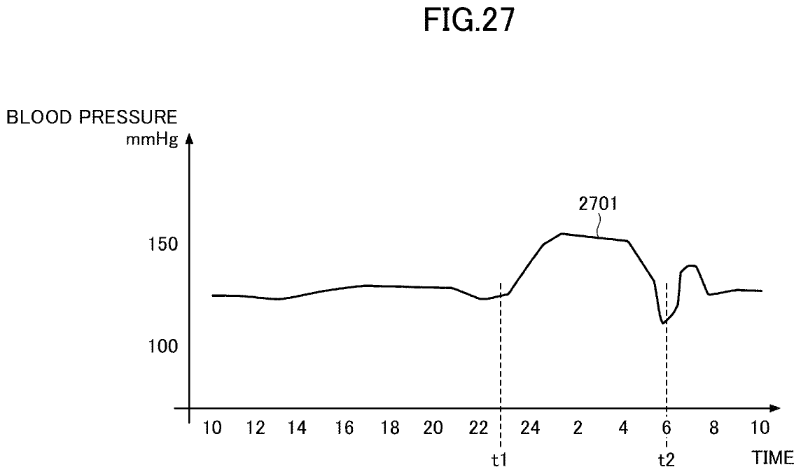

Next, the displaying at the medical institution is described. The terminal of the medical institution can display time-series information (blood pressure waveform) of blood pressure measured by the sticker-type pulse wave sensor 2500 and stored on the cloud server 2401. FIG. 27 is a diagram illustrating time-series information of blood pressure measured by the sticker-type pulse wave sensor according to the present embodiment. FIG. 27 illustrates waveform data 2701 indicating changes in blood pressure in time-series order. Further, in the example illustrated in FIG. 27, event information indicating getting up after sleeping and the starting of sleep superimposed onto the blood pressure waveform. In other words, the time-series information illustrated in FIG. 27 indicates time t1 and time t2 at which the event information is registered. The time t1 is the time at which the test subject started sleeping, and the time t2 is the time at which the test subject got up after sleeping. The duration of time from time t1 to time t2 is the sleep time.

The terminal 2402 of the medical institution can recognize blood pressure during sleep by displaying event information (icons indicating sleep and getting up after sleeping) and time-series information of continuous blood pressure (waveform data 2701) in a superimposed manner. Thus, medical personnel can easily determine whether blood pressure during sleep is higher or lower than blood pressure during the daytime. Thus, the present embodiment can improve the accuracy of the 24 hour blood pressure diagnosis by the medical personnel.

Furthermore, the diagnostic system according to the present embodiment has a function of performing pattern matching.

The cloud server 2401 according to the present embodiment stores a waveform model (an example of a change model) representing a predetermined blood pressure waveform (a time-series change in blood pressure) in a storage unit (not illustrated) for each classification representing characteristics of sleep of the test subject during sleep time.

Then, the cloud server 2401 extracts the time-series changes in the blood pressure determined based on the acquired measurement information. Then, the cloud server 2401 superimposes the events (starting of sleep and getting up after sleeping) indicated by the event information onto the time-series changes in the blood pressure, and extracts the time-series changes in the blood pressure during sleep based on the events.

Then, the cloud server 2401 identifies the classification of the test subject by performing pattern-matching between the time-series changes in the blood pressure extracted during the sleep and the time-series change model of the blood pressure. The classification of the test subject identified by the cloud server 2401 is output to the terminal 2402 of the medical institution. By doing so, the terminal 2402 of the medical institution can display a result of the pattern-matching on the cloud server 2401.

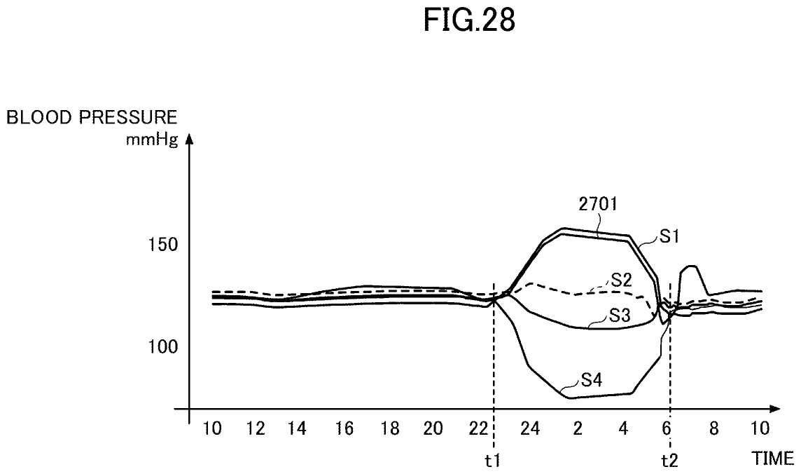

FIG. 28 is a diagram illustrating a classification result by pattern-matching with respect to the time-series information of blood pressure according to the present embodiment. The pattern-matching illustrated in FIG. 28 may be performed by the terminal 2402 of the medical institution. The result of the pattern-matching may be displayed on the terminal 2402 of the medical institution.

In the pattern-matching illustrated in FIG. 28, four types of time-series change models of blood pressure are stored. For example, the cloud server 2401 stores a Riser type change model S1, a Non-dipper type change model S2, a Dipper type change model S3, and an Extreme-dipper type change model S4. The four types of change models are illustrated as examples, and other change models may be included.

Further, the matching rate between the change in the time-series of the blood pressure of the test subject (waveform data 2701) by the cloud server 2401 and each of the four types of change models are calculated. The calculated matching rates are illustrated in Table 4.

TABLE 4

| | Subtype | Matching rates |
|---|---|---|
| S1 | Riser type | 0.8 |
| S2 | Non-dipper type | 0.5 |
| S3 | Dipper type | 0.1 |
| S4 | Extreme-dipper type | 0.01 |

Generally, a slightly lower blood pressure value during sleep is a healthy state, but a slightly higher change pattern (Riser type) or a pattern in which the blood pressure significant decreases (Extreme-dipper type) may become a health problem. The matching rates illustrated in Table 4 are calculated by the cloud server 2401, but may be calculated by another terminal such as the terminal 2402 of the medical institution. The matching rate may be calculated by any technique, for example, by using a correlation coefficient with a typical waveform.

Also, pattern-matching is not limited to changes in blood pressure in time-series during sleep. For example, in a case of early morning hypertension, the pattern-matching may be performed by patterning the timing with getting up after sleeping. When pattern-matching is performed, changes in blood pressure caused by going to the bathroom, going on a walk in the early morning, or exercising may affect the calculation of the matching rate. Therefore, when calculating the pattern-matching and the matching rates, the cloud server 2401 may exclude, the changes in the time series of the blood pressure of the test subject, the blood pressure fluctuations occurring at the same time as the event information affecting the pattern-matching, based on the event information described above from the change of the blood pressure of the test subject in the time-series.

### Eleventh Embodiment

The technique of the measurement of the blood pressure in the above-mentioned embodiment is described. However, the system described above may be used for purposes other than blood pressure measurement. Therefore, in the eleventh embodiment, for example, a state regarding stress of the test subject may be measured. For example, by holding a meeting with a plurality of subjects who are wearing the above-described sticker-type pulse wave sensors and performing measurements at the same time, stressors that cause stress can be objectively extracted, and interventions, such as the prompting of corrections, can also be implemented.

### <Measurement Technique>

A stress measurement technique is implemented by affixing a 24-hour pulse wave sensor to the test subject in the same manner as the 24-hour blood pressure measurement described above. In the measurement of stress or the like, points that differ the measurement of blood pressure in the above-described embodiment are described. The differences are measurement of cerebral blood flow, modeling of the test subject, event input function, and speaker identification function.

In a diagnostic system according to the present embodiment, measurement is basically performed while the test subject engages in his or her normal everyday-life activities. This is because the purpose of the diagnostic system according to the present embodiment is to capture, as data, a disease in which blood pressure fluctuates due to any psychological effects caused by an external stimulus, one example of such a disease being known as workplace hypertension. In the diagnostic system according to the present embodiment, the diagnosis result of the cloud server 2401 may be transmitted to a communication terminal installed at a workplace so that a type of diagnosis other than a medical diagnosis can be performed.

In the diagnostic system according to the present embodiment, the test subject is psychologically affected consequently altering the autonomic nervous system, a determination is made by using an element that changes blood pressure. With the diagnostic system according to the present embodiment, hypertension medication for the test subject can be optimized, and when the blood pressure of the test subject increases, it is possible to notify the test subject or someone else that the blood pressure has increased, and to make the test subject aware of the increased blood pressure, thereby enabling intervention in the control of the blood pressure.

### <Cerebral Blood Flow Measurement>

In this embodiment, the sticker-type pulse wave sensor 2500 is fixedly placed on the upper arm, and at the same time, another sticker-type pulse wave sensor is worn on the head.

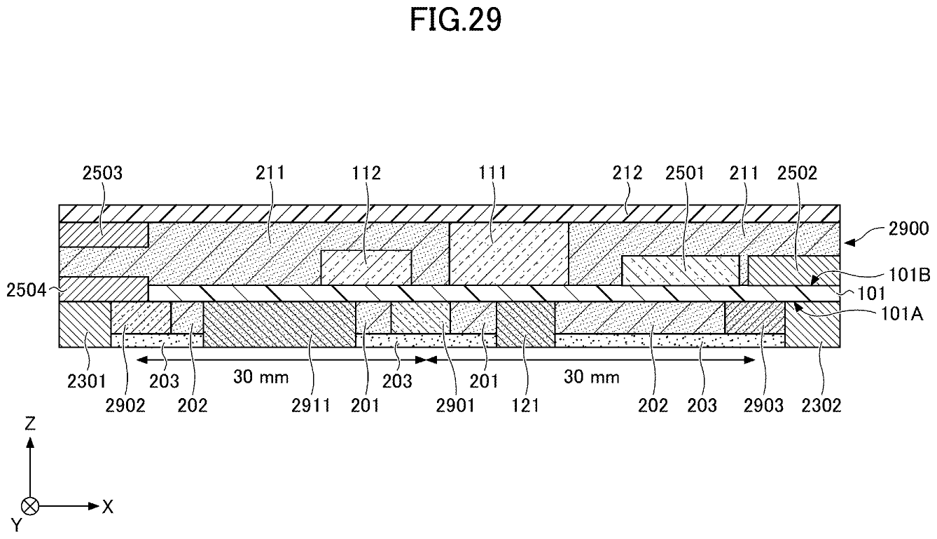

FIG. 29 is a cross-sectional view illustrating an optical system of a sticker-type pulse wave sensor 2900 according to the present embodiment. The components that are the same as those of the above-described sticker-type pulse wave sensor 2500 are denoted by the same reference numerals and descriptions are omitted. The sticker-type pulse wave sensor 2900 according to the present embodiment is intended to measure cerebral blood flow inside the skull of the head of the test subject.

The sticker-type pulse wave sensor 2900 is provided with surface-emitting lasers (VCSEL: Vertical-Cavity Surface-Emitting Laser) 2902 and 2903 in place of the LED 113. The surface-emitting lasers 2902 and 2903 output short-pulse laser beams. The sticker-type pulse wave sensor 2900 further includes a Single Photon Avalanche Diode (SPAD) 2901 as a detector. The sticker-type pulse wave sensor 2900 may have the LED 113 together with the surface-emitting lasers 2902 and 2903.

The distance from the surface-emitting lasers 2902 and 2903 to the SPAD 2901 is, for example, 30 mm. With this configuration, the light, which is output by the surface-emitting lasers 2902 and 2903, returning to the SPAD 2901 from the skin is minimized, and thus detection of cerebral blood flow occurring at the brain surface inside the skull is facilitated. Therefore, light shielding layer 2911 is provided between the surface-emitting laser 2902 and the SPAD 2901, and the shielding layer 121 is provided between the surface-emitting laser 2903 and the SPAD 2901.

The surface-emitting lasers 2902 and 2903 according to the present embodiment use, for example, a thin oxide confinement layer in order to keep a light confinement coefficient referred to as “gamma switch” low. Specifically, the surface-emitting lasers 2902 and 2903 are characterized in that a low refraction region is formed by oxide confinement, the thickness of the high refraction region that is not oxidized is 35 nm or less, and the thickness of the low refraction region at a position 3 μm from the tip of the boundary between the low refraction region and the high refraction region is 2 times or less the thickness of the high refraction region. By having this feature, the surface-emitting lasers 2902 and 2903 can stabilize sub-nanosecond short-pulse light emission. Wavelengths at which there is little optical absorption by water yet there is absorption by hemoglobin, such as 780 nm and 805 nm, are selected as the wavelengths at which light is emitted by the surface-emitting lasers 2903 and 2903. Further, as the wavelength of light emitted by the surface-emitting lasers 2902 and 2903, a wavelength of 940 nm at which the light absorption by water is small or a wavelength of 870 nm at which the detection sensitivity of the SPAD 2901 is high may be selected.

The SPAD 2901 is a light receiving unit having a sensor that is a single photon detecting element. The SPAD 2901 includes photoelectric conversion units (not illustrated) having a photoelectric conversion element for receiving light; a pulse generation unit (not illustrated) to generate a pulse signal corresponding to the amount of light received by the photoelectric conversion element; and a bit counter unit (not illustrated) to count the pulse signals. The bit counter unit is provided in each of the photoelectric conversion units in a distributed manner. By increasing the aperture ratio of the light-receiving region of each photoelectric conversion unit, the sensitivity of the SPAD 2901 is increased, and thus minute light diffused in the brain can be detected.

The sticker-type pulse wave sensor 2900 according to the present embodiment is affixed to three positions on the head of the test subject.

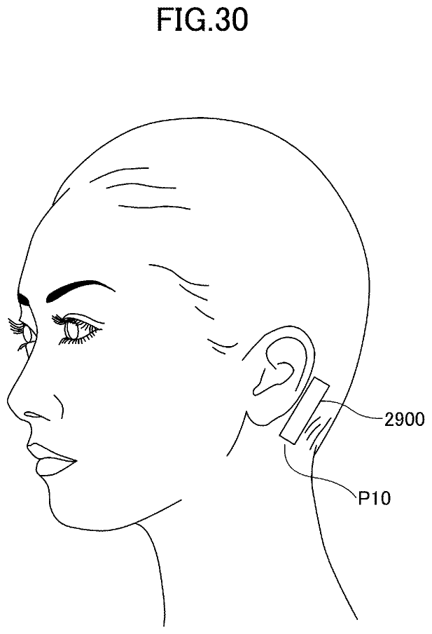

FIG. 30 is a diagram illustrating an example in which the sticker-type pulse wave sensor 2900 according to the present embodiment is affixed to the head of the test subject at a position behind the ear (first affixing position P10). As illustrated in FIG. 30, a sticker-type pulse wave sensor 2900 is affixed to the head of the test subject at a position behind ear, specifically, to a position where the temporal lobe can be measured. By affixing at the first affixing position P10 illustrated in FIG. 30, an occurrence of discomfort even in a workplace environment can be alleviated.

The temporal lobe, illustrated in FIG. 30, is the portion related to language which is the most important aspect in meetings and so on. When this portion is activated, it can be assumed that the person is concentrating on the meeting.

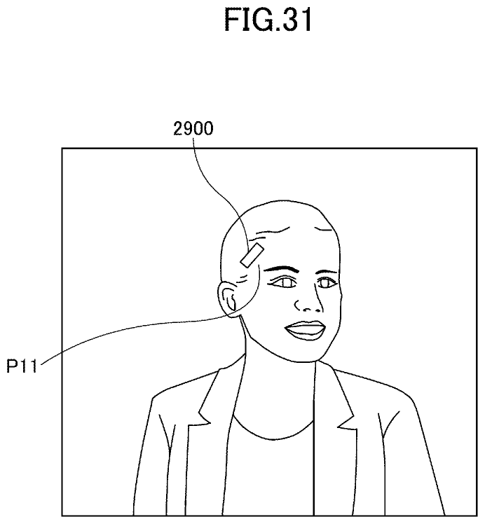

FIG. 31 is a diagram illustrating an example in which the sticker-type pulse wave sensor 2900 according to the present embodiment is affixed to the left temple of the test subject (second sticking position P11). The left temple, which is the second pasting position P11, is related to the dorsolateral prefrontal cortex (DLPFC) and is the portion that has a brain function that is highly correlated with depression, motivation, or the like. Therefore, the diagnostic system is effective in determining depression or the motivation of the test subject based on the measurement information from the sticker-type pulse wave sensor 2900 affixed to the second affixing position P11 (For example, Sachiyo Ozawa, Kazuo Hiraki, “Negative Emotion Regulation Using Working Memory Task and Finger Tapping: A Near-infrared Spectroscopy (NIRS) Study” (CD-ROM) (Program and Abstract Collection (CD-ROM) of the Japanese Cognitive Science Society), 2014, Vol. 31, pp. 1 to 4.). If the diagnostic system determines that the particular portion of test subject is activated, it can be assumed that the test subject is working to visualize a solution to the stressful problem in himself or herself based on his or her past memory. In other words, the diagnostic system may determine that the test subject is committed to the agenda of the meeting and is concentrating on the agenda issues.

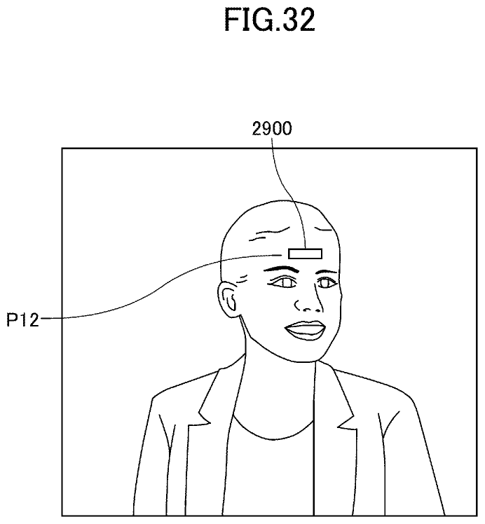

FIG. 32 is a diagram illustrating an example in which the sticker-type pulse wave sensor 2900 according to the present embodiment is affixed between the eyebrows of the test subject (third affixing position P11). The area between the eyebrows, which is the third pasting position P12, is a portion reflecting the entire frontal lobe, in particular the orbitofrontal cortex (OFC) and dorsomedial prefrontal cortex (DMPFC). Therefore, the diagnostic system can determine the degree of social cognition of the test subject based on the measurement information from the sticker-type pulse wave sensor 2900 affixed to the third attachment position P12. For example, during a time period of heightened social cognition, it can be determined that feelings where emphasis is placed on teamwork are heightened. In the diagnostic system, measurement information from the sticker-type pulse wave sensor 2900 affixed to the third attachment position P12 is useful in understanding the condition of the test subjects, such as the feelings of the test subject, as well as the pulse and blood pressure reflecting the autonomic nervous system of the test subject.

In the diagnostic system according to the present embodiment, the affixing positions of the sticker-type pulse wave sensor 2900 are merely examples, and as such, the affixing positions are by no means a limitation. In addition, although the present embodiment describes an example in which three sticker-type pulse wave sensors 2900 are affixed, the number of sticker-type pulse wave sensors 2900 to be affixed is not limited, and as such, the number may be two or less or four or more.

### <Event Detection>

The sticker-type pulse wave sensor 2900 according to the present embodiment includes a microphone 2502. The microphone 2502 may be configured as a package of several millimeters, for example, by MEMS technology. Such an extremely small microphone 2502 is mounted on the flexible printed circuit board 101. The vocal sounds collected by the microphone 2502 may be stored in the storage unit 116 or may be stored in the communication terminal 2411 on the cloud server 2401 via the wireless communication unit 115. This makes it easy to confirm what has happened when the condition (e.g., blood pressure) of the test subject suddenly changes.

A cloud server 2401 accumulates voice data collected by the microphone 2502 from the sticker-type pulse wave sensor 2900 provided by each of the test subjects through the communication terminal 2411, and analyzes the accumulated voice data. In the analysis, for example, it is determined which of the test subjects the speaker of the vocal sounds (hereinafter, also referred to as voice) included in the voice data is. Any technique may be used for associating the voice with the test subject, including well-known techniques. For example, the speaker may be identified by performing similarity determination with the voice data registered in advance for each test subject.

In this embodiment, the cloud server 2401 identifies the event number related to the conversation based on the received measurement information and various types of information of the sticker-type pulse wave sensor 2900. However, in the present embodiment, the method of identifying the event number is not limited to the method where identification is performed by the cloud server 2401, and as such, the event number may be input from a test subject or the like via the communication terminal 2411.

The cloud server 2401 identifies the event number of the test subject based on the voice corresponding to the test subject and the measurement information of the test subject. Some of the identified event numbers are illustrated in Table 5. As illustrated in Table 5, even the event is determined to be a meeting, the event number corresponding to the test subject may be identified from among meeting A (lots of speech), the meeting B (little speech), and the meeting C (no speech) in accordance with the amount of speech. In addition, the cloud server 2401 identifies an event number corresponding to the content of work such as office work.

TABLE 5

| Event Number | Event |
|---|---|
| 101 | Meeting A (lots of speech) |
| 102 | Meeting B (little speech) |
| 103 | Meeting C (no speech) |
| 104 | Conversation (user A) |
| 105 | Conversation (user B) |
| 106 | Conversation (user C) |
| 107 | Office work |
| 108 | Music |
| 109 | TV |
| 110 | Reading |
| 111 | Stretching |
| 112 | Cardiovascular exercise |
| 113 | Weight training |

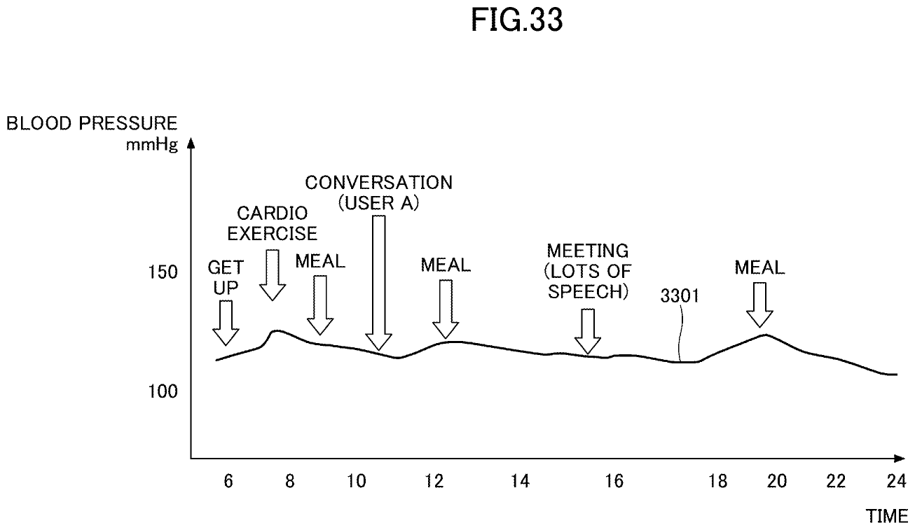

FIG. 33 is a diagram illustrating a result of measuring changes in blood pressure of a test subject and an example of an events that occurred with respect to the test subject. In the example illustrated in FIG. 33, the cloud server 2401 identifies an event number (refer to Table 3 and Table 5) corresponding to an event occurring with regard to the test subject based on voice data and measurement results, as well as fluctuations 3301 in the blood pressure of the test subject over a period of 24 hours.

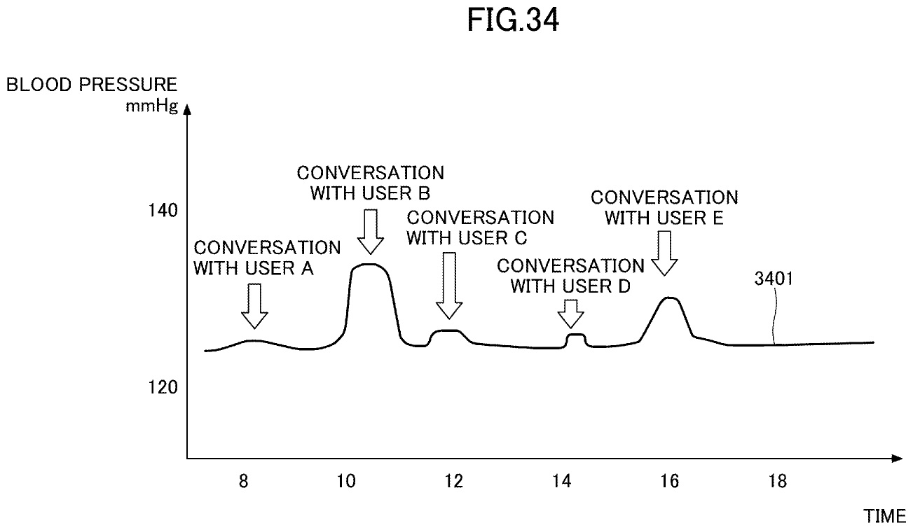

FIG. 34 is a diagram illustrating an example in which only the events where the test subject talked are extracted by the cloud server 2401 according to the present embodiment. In the example illustrated in FIG. 34, the cloud server 2401 corrects blood pressure fluctuations caused by events other than the conversations in order to identify and extract the events where there was conversation and then creates a graph. This facilitates the relative comparisons between the speakers.

Then, a communication terminal provided in the workplace displays a screen illustrating the determination result of the cloud server 2401. On this screen, a score obtained by converting a blood pressure fluctuation range may be displayed together with events and blood pressure fluctuations illustrated in FIG. 34. For example, a score indicating the rate of increase from the average blood pressure may be displayed on this screen. For example, in a case where an average blood pressure (compressed blood pressure) of 120 mmHg increased to 125 mmHg due to conversation, the increase rate is about 4%, so the score is 0.04. The score is calculated by the cloud server 2401.

In other words, the cloud server 2401 acquires multiple blood pressure waveforms representing the blood pressure of the test subject (body of the test subject) in the time series for each test subject from the measurement information acquired by the sticker-type pulse wave sensors 2500 and 2900. Further, the cloud server 2401 acquires an event number (example of conversation event information) indicating the timing of conversation with another person for each test subject from the voice data acquired together with the measurement information.

For each user (the test subject, for example) illustrated in FIG. 34, the cloud server 2401 calculates a score determined based on a change in blood pressure occurring at the timing of a conversation with another user (for example, another test subject), the timing of the conversation being indicated by an event number (example of conversation event information). Table 6 illustrates the scores calculated for each user illustrated in FIG. 34.

TABLE 6

| Speaker Number | Speaker | Score |
|---|---|---|
| 1 | User A | 0.04 |
| 2 | User B | 0.1 |
| 3 | User C | 0.05 |
| 4 | User D | 0.03 |
| 5 | User E | 0.08 |

In the present embodiment, an example is given in which a one-on-one conversation is performed when the variation in blood pressure during conversation is measured. Although it is preferable that the test subjects be at rest before and during the conversation, the state of the test subjects and the state of the conversation are not limited, and as such any state may be used.

By calculating the score, the it is possible to objectively recognize which test subject's conversation causes an increase in blood pressure. In the present embodiment, by using the same technique, the stress level with respect to an event such as a conference or an interview can be compared and verified.

In the present embodiment, each of test subjects is equipped with the sticker-type pulse wave sensors 2500 and 2900. For example, one manager and several staff members of the manager may wear the same sticker-type pulse wave sensors 2500 and 2900 at the same time. In this case, individual interviews may be conducted between the manager and each staff member in a one-on-one format. In this embodiment, the blood pressure variation of each interview participant is calculated as a score. The calculation results are illustrated in Table 7.

TABLE 7

| Interview Number | Interview Participant | Score |
|---|---|---|
| 1 | User A | 0.01 |
| 2 | User B | 0.2 |
| 3 | User C | 0.02 |
| 4 | User D | 0.04 |
| 5 | User E | 0.01 |

In the example illustrated in Table 7, the terminal provided in the workplace can display the calculation results illustrated in Table 7. Since the conversation scores of the staff members can be compared, the manager knows who is likely to be stressed. Thus, the manager can hand over a task suitable for each staff member. In addition, staff members can objectively recognize that they are inflicting stress or experiencing stress by making an average comparison with other colleagues. Staff members can also encourage behavioral changes in their manager and improve the well-being index of the work environment.

Further, in the diagnostic system according to the present embodiment, the stress generated in the workplace environment can be quantified, such as the score at the time of conversation, even in a flat-organizational relationship rather than the manager-staff member relationship. Therefore, for example, all five test subjects working in the workplace wear the same sticker-type pulse wave sensors 2500 and 2900. The diagnostic system measures all five people for 24 hours. During the measurement, test subjects are allowed to converse with each other as appropriate while performing their usual duties. The diagnostic system acquires blood pressure fluctuations during conversation. Thus, a score for each speaker illustrated in Table 6 is generated for each test subject. Then, the cloud server 2401 can quantify the person who inflicts stress on the other test subjects out of the five test subjects based on the scores of the five test subjects. For example, after adding up and averaging the scores calculated for interview participant, the interview participant with the highest score can be identified as the stressor. Table 8 illustrates the scores calculated for each test subject. In the example illustrated in Table 8, user B has the highest score and can therefore be identified as the stressor.

TABLE 8

| Test Subject Number | Test Subject | Score |
|---|---|---|
| 1 | User A | 0.01 |
| 2 | User B | 0.2 |
| 3 | User C | 0.02 |
| 4 | User D | 0.04 |
| 5 | User E | 0.01 |

### <Performing Corrections>

The score described above is a numerical value of the blood pressure increase rate of the test subject. In some cases, however, the rate of increase in blood pressure does not correspond exactly with the degree of stress. Therefore, the cloud server 2401 according to the present embodiment may correct the score by referring to the measurement result of the cerebral blood flow of the test subject. Any technique may be used as the correction technique.

For example, the DLPFC located at the temple is known to act to alleviate stress. For this reason, when it is determined that the test subject is experiencing stress, the cloud server 2401 corrects the score in consideration of the stress by regarding that stress-induced alleviation is working when the cerebral blood flow near the temple is increased. For example, the cloud server 2401 performs correction by, for example, dividing the calculated score by 0.9 or so on.

As another example, when cerebral blood flow is rising in the prefrontal area, such as between the eyebrows, concentration is improved and blood pressure is likely rising. Therefore, the cloud server 2401 determines that there is a high possibility that the increase in the blood pressure is not only due to the effect of the interview participant, and performs correction by multiplying the calculated score by 0.9.

The technique of correcting the score is not limited to the above-described techniques, and as such, machine learning may be employed, for example. Also, if the interview participant has a high position in the company, the other interview participant inevitably feels stress. In such a case, the cloud server 2401 may perform the correction according to a model inputted in advance. For example, the cloud server 2401 may perform correction according to a personality model based on personnel information inputted in advance. Next, the correction according to the model is described.

### <Correction by Modeling>

The cloud server 2401 receives input of information indicating a job title in the company and a social position as a model of a manager and a test subject for which correction is to be performed from a communication terminal provided in a workplace. The cloud server 2401 may store the job description of the supervisor and the test subject in a database. The cloud server 2401 may receive a selection from a test subject from a list of job descriptions stored in a database through a communication terminal provided in a workplace.

In the diagnostic system according to the present embodiment, the profiling and modeling of the test subject and the manager are facilitated by the configuration selected from the choices. A variety of information is input to generate a model, including company job title, job description, and coworker organizational makeup. The cloud server 2401 models the test subject from the information that is input. In the present embodiment, a biological model and a social model are generated. In the diagnostic system according to the present embodiment, the modeling is not limited to the above-described model, and a personality model may be generated.

### <Biological Model>

In the biological model, as biological parameters of the test subject, for example, age, gender, weight, height, blood test value (cholesterol level), uric acid value, and underlying disease (optional) are each quantified.

### <Social Model>

In the social model, each of company information, job type, job title, number of team members, related people, voice information, and personnel information is appropriately processed and modeled as social parameters of the test subject and the manager. In the social model, a job defined as a job type may be used.

<Personality Model>

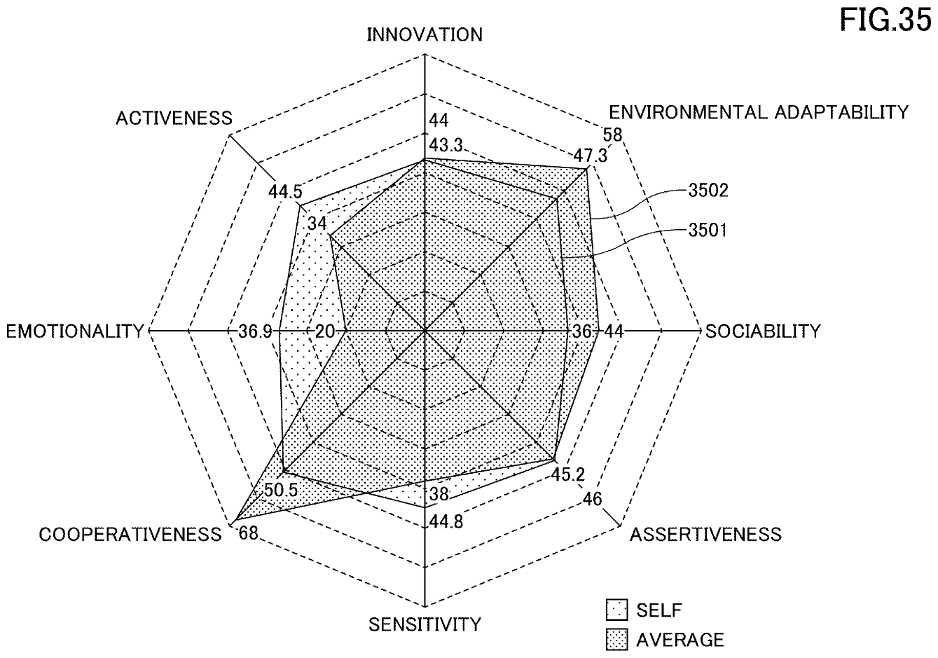

The personality model is generated by referring to subjective-centered information based on a questionnaire survey as personality parameters of the test subject and the manager. FIG. 35 is a diagram illustrating a personality model of a test subject. FIG. 35 illustrates a personality model 3502 of the test subject and an average personality model 3501. In the example illustrated in FIG. 35, innovativeness, activeness, emotionality, cooperativeness, sensitivity, assertiveness, sociability, and environmental adaptability are illustrated as parameters indicating personality, but other parameters may be used as the parameters indicating personality. In the diagnostic system according to the present embodiment, the personality of the test subject and the supervisor may be classified in comparison with the average value illustrated in FIG. 35.

The cloud server 2401 corrects the score of the test subject who conversed with the manager, according to the model (for example, the social model or the personality model) corresponding to the manager. Therefore, the cloud server 2401 retains the correction values or the correction formulas. The correction values are determined based on the social model of the manager and the paper-based personality model. Further, the cloud server 2401 corrects the scores calculated using the models (for example, the biological model, the social model, and the personality model) of the test subject. Therefore, the cloud server 2401 retains the correction values determined based on the biological model, the social model, and the paper-based personality model of the test subject or retains correction formulas. It should be noted that the specific correction values and the correction formulas are determined based on the embodiment, and as such, descriptions thereof are omitted.

Although an example is described in which the cloud server 2401 performs the calculation of the score, the correction of the score, and the classification of the personality, the technique performed by the cloud server 2401 is not limited. For example, a communication terminal provided in a workplace may calculate a score and correct the score.

### <Intervention>

In the present embodiment, an example is described in which a physical condition of a test subject, including stress in a workplace environment, is measured. In the workplace environment, physical condition may change due to stress or the like. In this case, the diagnostic system according to the present embodiment may alert the test subject to a change in physical condition. For example, workplace hypertension can be consciously corrected by the individual. Therefore, in the diagnostic system according to the present embodiment, the sticker-type pulse wave sensors 2500 and 2900 detect a change in the stress or blood pressure of the test subject, and prompt the test subject to change his/her behavior to improve the stress or blood pressure at the timing at which the change is detected.

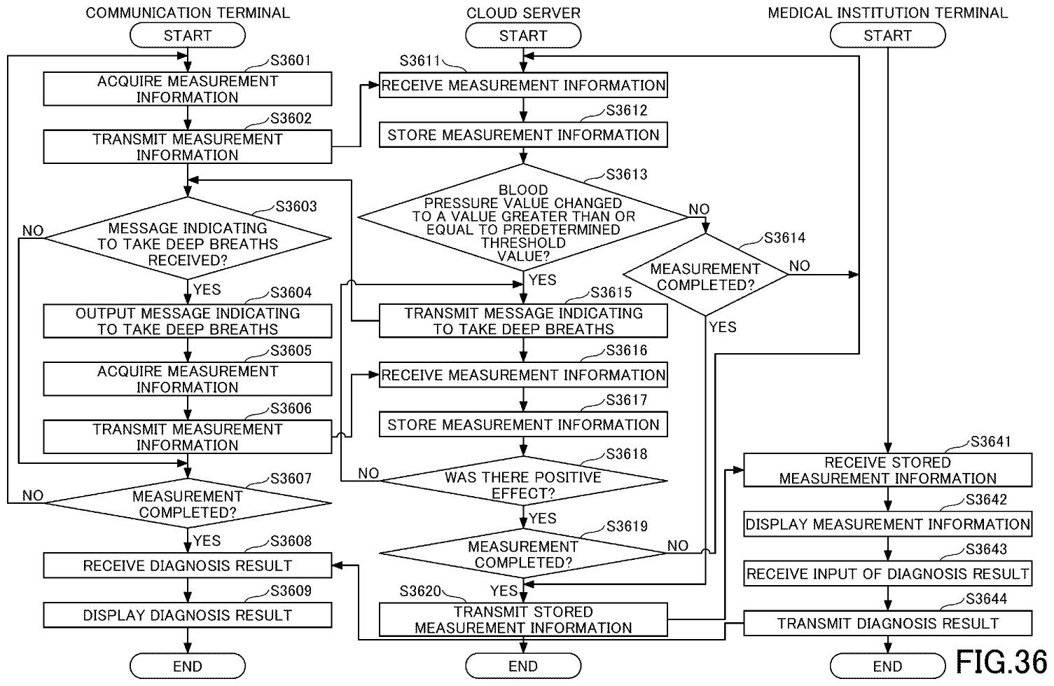

FIG. 36 is a flowchart illustrating processing performed by the diagnostic system according to the present embodiment. In the diagnostic system illustrated in FIG. 36, processing for encouraging a behavioral change in a test subject is illustrated. In the example illustrated in FIG. 36, it is assumed that the sticker-type pulse wave sensors 2500 and 2900 have already been fixedly placed on the test subject.

First, the communication terminal 2411 acquires measurement information of the test subject from the sticker-type pulse wave sensors 2500 and 2900 (S3601). Then, the communication terminal 2411 transmits the acquired measurement information to the cloud server 2401 (S3602).

Then, the cloud server 2401 receives the measurement information of the test subject from the communication terminal 2411 (S3611). The cloud server 2401 stores the received measurement information of the test subject in the storage device of the cloud server 2401 (S3612).

Then, the cloud server 2401 determines whether or not the blood pressure has changed to a value greater than or equal to a predetermined threshold based on the stored measurement information of the test subject (S3613). When it is determined that there is no change to a value greater than or equal to the predetermined threshold value (NO in S3613), it is determined whether or not the measurement of the test subject is completed (24 hours have passed since the measurement was started) (S3614). If it is determined that the measurement is not completed (NO in S3614), the cloud server 2401 performs the processing again from S3611.

Conversely, if the cloud server 2401 determines that the measurement is completed (YES in S3614), the processing proceeds to S3620.

In S3613, if it is determined based on the stored measurement information of the test subject that the blood pressure has changed to a value greater than or equal to a predetermined threshold (YES in S3613), the cloud server 2401 transmits a message to the communication terminal 2411 indicating that the test subject should take deep breaths (S3615).

Then, the communication terminal 2411 determines whether or not a message indicating to take deep breaths has been received (S3603). If it is determined that no reception has been made (NO in S3603), the processing proceeds to S3607.

Conversely, if the communication terminal 2411 determines that the message indicating to take deep breaths has been received (YES in S3603), the communication terminal 2411 outputs a message indicating to take deep breaths (S3604). The technique of outputting the message may be displayed on the screen of the communication terminal 2411 or the message may be output as voice as long as the message can be understood by the test subject, for example.

Thereafter, the communication terminal 2411 acquires measurement information of the test subject from the sticker-type pulse wave sensors 2500 and 2900 (S3605), and transmits the acquired measurement information to the cloud server 2401 (S3606).

The cloud server 2401 receives the measurement information of the test subject from the communication terminal 2411 (S3616), and stores the received measurement information of the test subject in the storage device of the cloud server 2401 (S3617).

Then, the cloud server 2401 determines whether or not the message had positive effect on the test subject based on the stored measurement information of the test subject (S3618). If it is determined that the message had no positive effect (NO in S3618), the processing starts again from S3615.

Conversely, if it is determined, based on the stored measurement information of the test subject, that the message had a positive effect (YES in S3618), the cloud server 2401 determines whether or not the measurement of the test subject is completed (24 hours have passed since the measurement was started) (S3619). If it is determined that the measurement is not completed (NO in S3619), the cloud server 2401 performs the processing again from S3611.

Conversely, if the cloud server 2401 determines that the measurement is completed (YES in S3619), the processing proceeds to S3620.

After the measurement is completed, the cloud server 2401 transmits the stored measurement information to the terminal 2402 of the medical institution thereby completing the processing (S3620). When transmitting the measurement information, the cloud server 2401 performs the above-described corrections or the like on the measurement information.

The terminal 2402 of the medical institution receives the measurement information from the cloud server 2401 (S3641). The terminal 2402 of the medical institution displays the received measurement information (S3642).

Then, the terminal 2402 of the medical institution receives the input of the diagnosis result to the test subject from the medical personnel (S3643).

Then, the terminal 2402 of the medical institution transmits the diagnosis result to the communication terminal 2411 (S3644).

In S3607, the communication terminal 2411 determines whether or not the measurement of the test subject is completed (24 hours have elapsed since the measurement was started) (S3607). If the communication terminal determines that the measurement is not completed (NO in S3607), the communication terminal 2411 performs the processing again from S3601.

Conversely, if the communication terminal determines that the measurement of the test subject is completed (24 hours have passed since the measurement was started) (YES in S3607), the communication terminal 2411 receives the diagnosis result from the terminal 2402 of the medical institution (S3608).

Then, the communication terminal 2411 displays the received diagnosis result (S3609).

In the present embodiment, the physical condition of the test subject can be improved by encouraging deep breathing when the blood pressure of the test subject changes by performing the above-described processing. In addition, by grasping such a change in blood pressure by the medical personnel of the medical institution, the test subject can be urged to take more appropriate action after the measurement is completed.

### Twelfth Embodiment

In the above-described embodiment, an example in which a medical institution sends a sticker-type pulse wave sensor to a test subject has been described. However, the sending source of the sticker-type pulse wave sensor is not limited to a medical institution. Therefore, the twelfth embodiment describes an example in which a pharmacist sends the sticker-type pulse wave sensor to the test subject.

In this embodiment, the oxygen saturation level of hemoglobin can be measured based on the measurement information of the sticker-type pulse wave sensor 2500. The service related to the home-based medical treatment can be realized by this.

As illustrated in FIG. 26, the sticker-type pulse wave sensor 2500 according to the present embodiment includes an LED that outputs a wavelength of 780 nm and an LED that outputs a wavelength of 850 nm as the LEDs 113. The sticker-type pulse wave sensor 2500 can detect the optical absorption coefficient of a living body at each of the wavelengths by causing the light emission timing of the LED that outputs a wavelength of 780 nm and the LED that outputs a wavelength of 850 nm to be different from other.

It is well known that the two kinds of wavelengths, 850 nm and 780 nm, are wavelengths that saliently represent the difference in the spectrum in the redox reaction of hemoglobin. Therefore, in the diagnostic system according to the present embodiment, the oxygen saturation level of the hemoglobin of the test subject can be detected from the absorption coefficients of the two types of wavelengths by reversely calculating the absorption coefficients of the known redox reaction.

For example, in a case where a test subject may have a highly contagious viral disease such as COVID-19, there is a possibility that a person who has been in contact with the test subject may become infected, so online diagnosis and home treatment are desired. In this case, the sticker-type pulse wave sensor 2500 may be used as an oxygen saturation level meter at the discretion of the physician by the online diagnosis in order to perform a specific measurement of the test subject.

In other words, by hospitalizing a test subject who may have a disease, medical personnel (e.g., a physician or nurse) can ascertain the situation of the test subject or provide advanced medical care to the test subject. However, it is costly because of the need for sophisticated virus protective measures. Therefore, it is desirable to create a situation where a detailed understanding of the current situation can be ascertained through use of an online diagnosis and home-based medical treatment. Therefore, in the diagnostic system according to the present embodiment, the sticker-type pulse wave sensor 2500 is used. Thus, the vital amount of at least one of the body temperature, pulse rate, pulse waveform, blood pressure, or respiratory rate of the test subject can be continuously monitored for 24 hours. Further, the cloud server 2401 transmits the vital amount of the test subject to the terminal 2402 of the medical institution, so that the physician belonging to the medical institution can appropriately determine the condition of the test subject based on the vital amount.

In particular, in the case of COVID-19, particular importance is placed on hemoglobin oxygen saturation level as an index by which progression of pneumonia in the test subject can be determined. The diagnostic system according to the present embodiment monitors the hemoglobin oxygen saturation level of the test subject for 24 hours. Thus, the medical personnel of the medical institution can appropriately determine the condition of the lungs of the test subject from the vital amount transmitted from the cloud server 2401. In addition, if the medical personnel determines that the test subject is in dangerous condition, the test subject can cause the medical personal can effect change in the behavior of the test subject to be hospitalized, for example, even if the test subject is not in the position to make appropriate judgment. Thus, in the diagnostic system according to the present embodiment, the danger in home-based medical treatment of the test subject is reduced, and thus safe home-based medical treatment can be achieved.

The diagnostic system according to the present embodiment is not limited to home-based medical treatment, and is effective in providing safe medical treatment, for example, in a facility such as a retirement home where not staffed with on-site physicians and nurses. Since the sticker-type pulse wave sensor 2500 according to the present embodiment is disposable, there is no problem of virus infection caused by the reuse of the sphygmomanometer, and the risk and cost incurred for the cleaning to prevent virus infection can be reduced. Measurement information detected by the sticker-type pulse wave sensor 2500 is stored in the cloud server 2401, and diagnosis is performed at the terminal 2402 of the medical institution. As described above, in the present embodiment, since it is not necessary to return the sensor or the diagnostic device to the medical institution or the like, the time and energy put into sending the sensor or the diagnostic device can be alleviated, and the risk associated breakdown during use can be reduced.

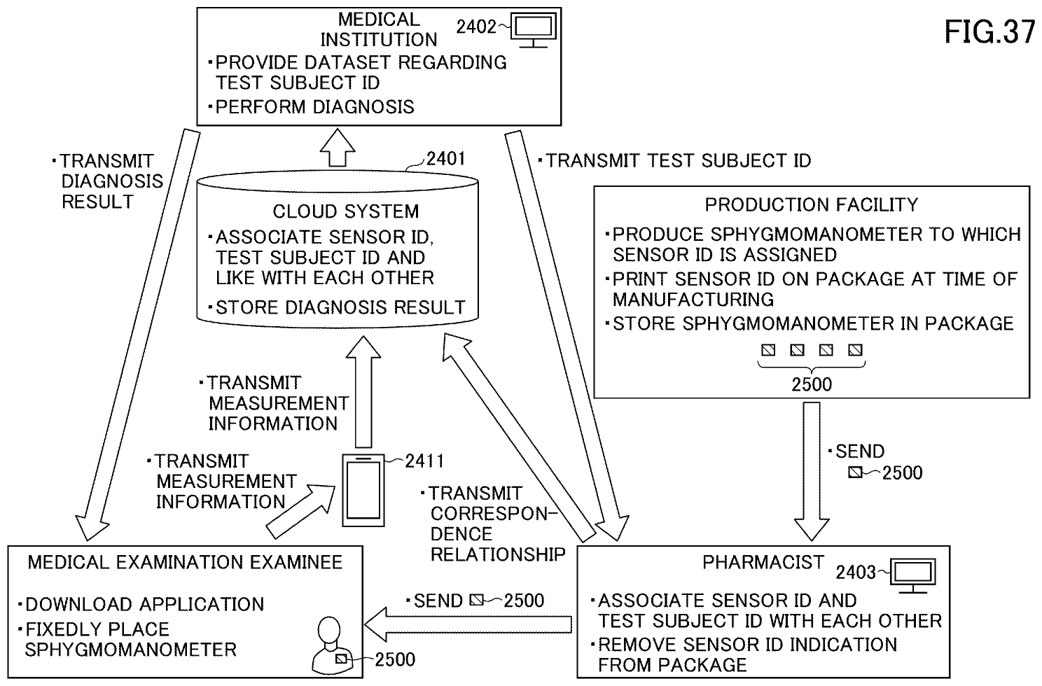

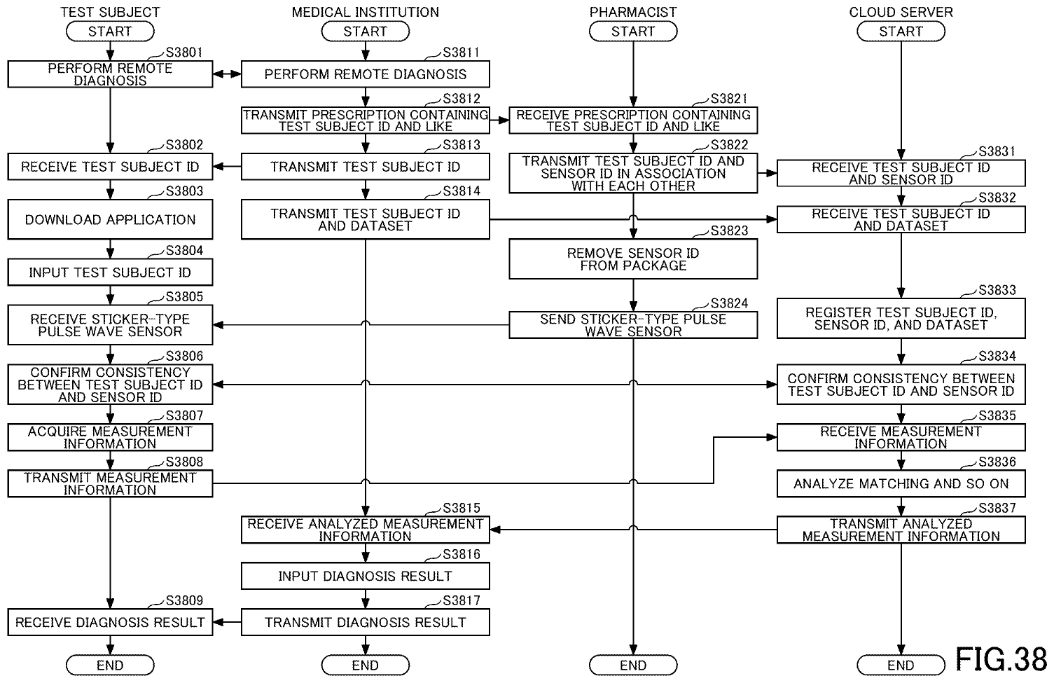

FIG. 37 is a diagram illustrating a diagnostic system to be used with the diagnosis service according to the present embodiment. In the diagnostic system according to the present embodiment, a (disposable) sensor ID uniquely allocated to each of the disposable sticker-type pulse wave sensors 2500 described above is used.

As illustrated in FIG. 37, the diagnostic system includes a production facility, a pharmacist, a medical institution, a test subject, and the cloud server 2401.

### <Manufacture of Sticker-Type Pulse Wave Sensor>

The production facility according to the present embodiment produces the sticker-type pulse wave sensor 2500 to which a sensor ID is assigned. Specifically, the production facility writes a sensor ID that uniquely identifies the sticker-type pulse wave sensor 2500 when writing a program to the storage unit 116 of the control device 112 by using a connection terminal (not illustrated) to the outside formed on the flexible printed circuit board 101 of the sticker-type pulse wave sensor 2500.

The production facility prints the sensor ID on the package at the time of manufacturing of the sticker-type pulse wave sensor 2500. The production facility stores the sticker-type pulse wave sensor 2500 in the package. The sensor ID of the package is printed at a position that can be read by a person managing inventory, such as a pharmacist.

Then, the production facility sends the sticker-type pulse wave sensor 2500 stored in the package to the pharmacist.

A physician belonging to the medical institution conducts online diagnosis with the test subjects. If the physician determines that an examination is necessary based as per the online diagnosis, the physician sends the pharmacist a prescription containing information about the test subject to whom the sticker-type pulse wave sensor 2500 is to be sent. In the case of the online diagnosis as in the present embodiment, the terminal 2402 of the medical institution may transmit the prescription as electronic information to a terminal 2403 of the pharmacist (pharmacy).

### <Inventory Management of Pharmacy>

The pharmacist provides the medicine to the test subject based on the prescription from the medical institution. The pharmacist also performs processing for sending the sticker-type pulse wave sensor 2500 to the test subject according based on the prescription.

The prescription the pharmacist receives contains the information of the test subject. The information of the test subject includes the test subject ID and the address of the test subject. In addition, the medical institution or ID of the physician may be provided. Therefore, the terminal 2403 can display the input screen of the prescription from the search screen on the diagnostic system according to the present embodiment using the ID of the medical institution or the physician as the search key. On the prescription input screen, the sensor ID of the sticker-type pulse wave sensor 2500 can be input. Thus, the test subject ID and the sensor ID of the sticker-type pulse wave sensor 2500 can be associated with each other.

The pharmacy manages the inventory of the sticker-type pulse wave sensors 2500 sent from the production facilities. Although inventory management is typically handled by a certified person, such as a pharmacist, the person responsible for inventory management is not limited to such a person. In the present embodiment, a description is provided for a case in which a pharmacist of a prescription pharmacy does the sending to the test subject, but a retail store such as a convenience store or a drug store may sell the sticker-type pulse wave sensor 2500 as is done for self-medication.

For example, the communication terminal (an example of the communication device) transmits to the cloud server 2401, in accordance with an operation from a certified person such as a pharmacist, a notification indicating that: the test subject ID indicating the test subject that is the sending destination; and the sensor ID of the sticker-type pulse wave sensor 2500 to be sent to the test subject, are to be registered in association with each other. When there is an ordering instruction for the sticker-type pulse wave sensor 2500 in the prescription of the physician, the cloud server 2401 can register: the test subject ID of the test subject; and the sensor ID of the sticker-type pulse wave sensor 2500 to be sent to the test subject, in association with each other. As a result, the registration of the sensor ID can be performed at the responsibility of the pharmacist in a reliable and safe manner, as is done for prescriptions of medicine. This ensures a highly safe and reliable diagnosis.

In this embodiment, in order to facilitate handling of electronic information by a pharmacist, it is preferable that an application that enables receiving of a prescription from a medical institution and inputting a test subject ID and a sensor ID of a sticker-type pulse wave sensor 2500 at the same time is installed in the terminal 2403 used by the pharmacist.

Thereafter, the pharmacist removes, from the communication terminal, both the test subject ID indicating the test subject that is the sending destination and the sensor ID printed on the package of the sticker-type pulse wave sensor 2500 to be sent to the test subject. By doing so, the sensor ID can be prevented from being redeployed by other people.

The pharmacist sends the sticker-type pulse wave sensor 2500 from which the sensor ID was removed to the test subject by way of a delivery person.

### <Delivery Person>

Online diagnosis has an advantage in that the patient can obtain medicine without any transmitting of a virus. In this embodiment, the sticker-type pulse wave sensor 2500 is sent using a delivery system similar to the online diagnosis. In this delivery system, it is desirable that a delivery person act as an intermediary in order to reduce the chance of virus transmission and reduce the burden on the test subject. The delivery person carries out the delivery in accordance with the instructions of the pharmacist.

The delivery instructions may be given from the terminal 2403 of the pharmacist. It is also desirable that the delivery person be a certified person assigned to the community, such as, for example, a community medical center employee, a health center employee, a pharmacist, or a public health nurse. If delivery is performed by a certified person who responsibly delivers the sticker-type pulse wave sensor 2500, the person delivering the sticker-type pulse wave sensor 2500 can assist the test subject in the fitting of the sticker-type pulse wave sensor 2500. This kind of service is desirable particularly for elderly people who need nursing care or test subjects who are in poor physical condition.

### <Response of the Test Subject>

The test subject downloads and installs an application for measuring the sticker-type pulse wave sensor 2500 to the communication terminal 2411 in advance. Thereafter, the communication terminal 2411 may display an input screen for inputting the attribute of the test subject, similarly to the above-described embodiment.

The test subject receives a sticker-type pulse wave sensor 2500 from a pharmacist. When receiving the sticker-type pulse wave sensor 2500 by a delivery person who has taken viral infection-prevention measures, the test subject inputs confirmation information, indicating that the sticker-type pulse wave sensor 2500 has been handed over to him/her, into a software application that was launched on the communication terminal 2411. By doing so, a physician, a pharmacist, or the like can confirm that the sticker-type pulse wave sensor 2500 has been delivered.

The test subject peels off the protective sheet 2303 from the sticker-type pulse wave sensor 2500. The first electrode 2301 and the second electrode 2302 are in contact with the protective sheet 2303. Therefore, when the test subject peels off the protective sheet 2303, the space between the first electrode 2301 and the second electrode 2302 becomes insulated. Thus, the control device 112 of the sticker-type pulse wave sensor 2500 can detect that the protective sheet 2303 has been peeled off. Accordingly, the control device 112 starts preparation for starting the measurement. That is, the sticker-type pulse wave sensor 2500 according to the present embodiment can reduce the consumption of the battery 111 by maintaining a sleep state until the protective sheet 2303 is peeled off. As a result, the sticker-type pulse wave sensor 2500 can reduce battery consumption up to the start of measurement, and thus can be configured in a compact design since having a low battery capacity is sufficient. Further, the battery 111 of the sticker-type pulse wave sensor 2500 can be made compact to improve the comfort when the sticker-type pulse wave sensor 2500 is worn.

Then, the test subject launches the application installed in the communication terminal 2411. Thereafter, wireless communication is established between the communication terminal 2411 and the sticker-type pulse wave sensor 2500. Any standard may be used as the wireless communication. For example, Bluetooth (registered trademark) may be used. When communication is established, the application may indicate so. Thus, the test subject can recognize that communication has been established.

The application of the communication terminal 2411 reads the sensor ID of the sticker-type pulse wave sensor 2500 stored in a storage unit 116. The application associates the already input test subject ID with the sensor ID of the sticker-type pulse wave sensor 2500. The communication terminal 2411 requests the cloud server 2401 as to whether or not the correspondence relationship is consistent. Then, the cloud server 2401 determines whether or not the correspondence relationship already registered matches and transmits the determination result to the communication terminal 2411. Then, the communication terminal 2411 displays the determination result.

The application of the communication terminal 2411 displays guidance such as the position where the sticker-type pulse wave sensor 2500 is to be affixed. For example, the test subject fixedly places the sticker-type pulse wave sensor 2500 on the upper arm, the clavicle, or the like in accordance with the guidance. As a result, the affixing position on the test subject is correct, so that accurate measurement can be performed.

For example, the test subject affixes the sticker-type pulse wave sensor 2500 to a position such as the upper arm in accordance with the guidance. The sticker-type pulse wave sensor 2500 can detect the resistance value of the skin by the first electrode 2301 and the second electrode 2302, and thus can recognize that the sticker-type pulse wave sensor 2500 is affixed to the skin of the test subject.

The sticker-type pulse wave sensor 2500 starts light emission of the LED 113, reading of the PD 114, and the like, in accordance with the recognition. The sticker-type pulse wave sensor 2500 starts biological measurement of the test subject, and upon starting detection of an appropriate pulse, transmits measurement information to the cloud server 2401 via the communication terminal 2411. Thus, a physician, a pharmacist or a delivery person can reliably confirm that the measurement has started. If the start of this measurement cannot be confirmed, a confirmation alarm may be issued by the physician, pharmacist, or delivery person. Thus, the application of the communication terminal 2411 of the test subject outputs an alarm sound and displays a screen for prompting measurement. In addition, the application of the communication terminal 2411 may output an alarm sound when the pulse wave of the test subject is not appropriately detected due to an inappropriate affixing position or the like. In this case, the communication terminal 2411 may transmit the alarm information to the terminal 2402 of the medical institution or the terminal of the pharmacist. If necessary, the communication terminal 2411 may provide guidance for attaching the sticker-type pulse wave sensor 2500 via a screen in cooperation with the video conference system. Thus, the application of the communication terminal 2411 outputs an appropriate instruction at an appropriate timing, so that accurate measurement can be performed.

### <Application on the Terminal of the Test Subject>

The application of the communication terminal 2411 of the test subject includes a function for transmitting and receiving information between the communication terminal 2411 and the cloud server 2401, a function for controlling the sticker-type pulse wave sensor 2500, and the like.

When the application is launched for the first time, the application receives input of information of the test subject. Specifically, the application receives input of the test subject ID transmitted from a medical institution. Furthermore, the application may receive the input of the name of the test subject, the name of the medical institution where the test subject visited, the address of the test subject, and the telephone number of the communication terminal 2411 (for example, a smartphone) being used. In addition, the application may receive input of various types of information such as a password, age, gender, weight, presence or absence of an underlying disease, details of the underlying disease, physical condition, temperature, and the like. For example, it is important for the address of the test subject to know where the test subject is currently recuperating. Therefore, information from GPS may be processed by the application taking into consideration places where the test subject is outside of the home including places hotels where the test subject may recuperate. Further, a method of collecting model information of the communication terminal 2411 by way of an opt-in may be adopted.

The communication terminal 2411 and the sticker-type pulse wave sensor 2500 are connected by radio communication.

Thereafter, that is, after starting the measurement, the sticker-type pulse wave sensor 2500 transmits, to the communication terminal 2411, information (hereinafter referred to as measurement information) indicating the measurement result.

The communication terminal 2411 (an example of the communication device) transmits measurement information including information on a pulse wave of a test subject calculated by the sticker-type pulse wave sensor 2500 to the cloud server 2401 together with a sensor ID of the sticker-type pulse wave sensor 2500. The cloud server 2401 stores measurement information received together with the sensor ID in association with the test subject ID corresponding to the sensor ID.

The cloud server 2401 transmits the received measurement information (information on the pulse wave of the test subject) and the test subject ID (associated with the sensor ID transmitted together with the measurement information) to the terminal 2402 of the medical institution.

The terminal 2402 of the medical institution receives the test subject information and the measurement information from the cloud server 2401, and performs displaying determined based on the received measurement information as received test subject information indicated by the test subject ID. The physician of the medical institution diagnoses the test subject based on the measurement information displayed on the terminal. Thereafter, the terminal 2402 of the medical institution transmits the diagnosis result to the communication terminal 2411 of the test subject. The measurement information used for diagnosis is the same as that in the above-described embodiments, and the description thereof is omitted.

### <Terminal of Medical Institution>

An application for implementing the diagnostic system is installed in a terminal 2402 of a medical institution. When the terminal 2402 receives the measurement information from the cloud server 2401, the terminal displays the measurement information together with an alarm sound to prompt the physician to perform a diagnosis.

Further, the terminal 2402 of the medical institution may receive a notice of completion indicating that the sticker-type pulse wave sensor 2500 is now worn by the test subject, notice of start of transmission of measurement information from the sticker-type pulse wave sensor 250, and the like, by the function of the installed application. Further, the terminal 2402 of the medical institution transmits a dataset including the biological information regarding the test subject to the cloud server 2401, so that the measurement information can be corrected as determined based on the biological information.

A terminal 2402 of a medical institution can receive measurement information including an analysis result from a cloud server 2401. In this case, the physician can make a diagnosis of the test subject based on a determination made taking into consideration the results of the analysis.

In the example illustrated in FIG. 37, the pharmacist registers the sensor ID and the test subject ID in the cloud server 2401 based on the prescription. Thus, the measurement information transmitted from the sticker-type pulse wave sensor 2500 can be associated with the test subject ID without the test subject performing the registration processing.

The pharmacist removes the sensor ID and sends the sticker-type pulse wave sensor 2500 to the test subject. Thus, since only the pharmacist can know the sensor ID, falsification or the like using the sensor ID can be suppressed to improve safety. In addition, as with other medicine, the physician can also check for prescription errors as the same time.

### <Measurement>

FIG. 38 is a flowchart illustrating processing performed by the diagnostic system according to the present embodiment. As illustrated in FIG. 38, first, online remote diagnosis is performed between the communication terminal 2411 of the test subject and the terminal 2402 of the medical institution (S3801 and S3811). The remote diagnosis may be a video conference using an image capturing apparatus or may be conducted by only voice.

The physician using the terminal 2402 of the medical institution determines whether or not the test subject is suspected to have COVID-19 or the like by the remote diagnosis. Although the flowchart illustrated in FIG. 38 illustrates an example where remote diagnosis is performed to determine whether or not there is a suspicion of COVID-19 or the like as remote diagnosis, it may be applied to remote diagnosis of other viral diseases. Furthermore, the remote diagnosis according to the present embodiment is not limited to the case of a viral disease, but may be applied to the diagnosis of a patient having an underlying disease such as a cardiac function or a patient who lives far away.

When the physician or the like determines that continuous 24 hour measurement of the vitals of the test subject is necessary, a prescription containing both the address of the test subject to be measured and the test subject ID of the test subject is transmitted to the terminal of the pharmacist via the terminal 2402 of the medical institution (S3812).

Then, the terminal 2403 of the pharmacist receives the prescription containing both the address of the test subject serving as the sending destination of the sticker-type pulse wave sensor 2500 and the test subject ID indicating the test subject who is the sending destination (S3821). The pharmacist has already received a sticker-type pulse wave sensor 2500 from a production facility.

Then, the terminal 2403 of the pharmacist transmits the test subject ID of the test subject in association with the sensor ID of the sticker-type pulse wave sensor 2500 to be sent to the test subject, and transmits the associated information to the cloud server 2401 (S3822), in accordance with the operation of the pharmacist.

The terminal 2402 of the medical institution sends the test subject ID to the communication terminal 2411 of the test subject in accordance with the operation of the physician or the like (S3813). Thereafter, the terminal 2402 of the medical institution transmits a test subject ID of the test subject and a dataset including biological data (for example, past examination results) of the test subject to the cloud server 2401, in accordance with the operation of the physician or the like (S3814).

The cloud server 2401 receives the test subject ID of the test subject and the sensor ID from the terminal 2403 of the pharmacist (S3831). Further, the cloud server 2401 receives the test subject ID of the test subject and the dataset from the terminal 2402 of the medical institution (S3832).

The cloud server 2401 registers the test subject ID of the test subject, the sensor ID, and the dataset in association with each other (S3833).

After transmitting the information to the cloud server 2401, the pharmacist removes the sensor ID printed on the package of the sticker-type pulse wave sensor 2500 (S3823). Thereafter, the pharmacist sends the sticker-type pulse wave sensor 2500 to the test subject (S3824).

After the remote diagnosis is performed, the communication terminal 2411 of the test subject receives the test subject ID (S3802). Thereafter, the communication terminal 2411 downloads and installs an application for performing 24 hour measurement, in accordance with the operation of the test subject (S3803). In order to download the application, an address of a site for downloading the application is provided by way of instruction from the physician or the like. For this instruction, sending means such as e-mail may be used. The test subject accesses the address by using the communication terminal 2411. This allows the application to be downloaded.

The communication terminal 2411 of the test subject receives the input of the test subject ID with respect to the installed application (S3804).

Thereafter, the test subject receives the sticker-type pulse wave sensor 2500 from the pharmacist (S3805). Thereafter, the test subject peels off the protective sheet 2303 from the sticker-type pulse wave sensor 2500. Thus, communication is established between the sticker-type pulse wave sensor 2500 and the communication terminal 2411. The communication terminal 2411 receives the sensor ID from the sticker-type pulse wave sensor 2500.

Then, the communication terminal 2411 of the test subject confirms the consistency between the test subject ID and the sensor ID with the cloud server 2401 (S3806 and S3834). The flowchart illustrated in FIG. 38 is assumed to be a case where there is consistency. Then, measurement by the sticker-type pulse wave sensor 2500 is started.

The communication terminal 2411 of the test subject acquires measurement information from the sticker-type pulse wave sensor 2500 (S3807). Thereafter, the communication terminal 2411 transmits the acquired measurement information to the cloud server 2401 (S3808). The processing in S3807 to S3808 is repeated for 24 hours.

Then, the cloud server 2401 receives, for a period of 24 hours, the measurement information from the communication terminal 2411 (S3835). The received measurement information is stored in a storage unit.

Thereafter, the cloud server 2401 performs analysis such as matching with respect to the stored measurement information (S3836). Specifically, the cloud server 2401 may model the test subject based on the input dataset of the test subject and correct the stored measurement information according to the model. Further, the cloud server 2401 may perform pattern-matching between time-series change model and the time-series changes by the measurement information. The cloud server 2401 may identify the classification corresponding to the test subject by pattern-matching. The time-series change model is a model is stored in advance and is for classifying the characteristics of the test subject. Further, in the present embodiment, as the characteristic of the test subject, a time-series change model in which the disease or the like of the test subject is classified may be prepared.

Then, the cloud server 2401 transmits measurement information including the analyzed information to the terminal 2402 of the medical institution (S3837).

The terminal 2402 of the medical institution receives measurement information including the analyzed information from the cloud server 2401 (S3815). The terminal 2402 of the medical institution displays the received measurement information or the like. Thus, the physician or the like can diagnose the test subject.

The terminal 2402 of the medical institution receives the input of the diagnosis result from the physician or the like (S3816).

Thereafter, the terminal 2402 of the medical institution transmits the diagnosis result from the physician or the like to the communication terminal 2411 (S3817).

The communication terminal 2411 of the test subject receives the diagnosis result from the terminal 2402 of the medical institution (S3809). Thus, the test subject can recognize the disease he or she has by referring to the diagnosis result.

In the remote diagnosis illustrated in FIG. 38, on-line diagnosis by the communication terminal is assumed, but voice-based diagnosis by the telephone or the like may be used, or on-line diagnosis using the existing cloud service may be used. When an online diagnosis using an existing cloud service is used, the online diagnosis service may have an application download function.

In the diagnostic system according to the present embodiment, an alarm function for the physician is provided. For example, if the test subject's condition deteriorates, the physician can immediately respond.

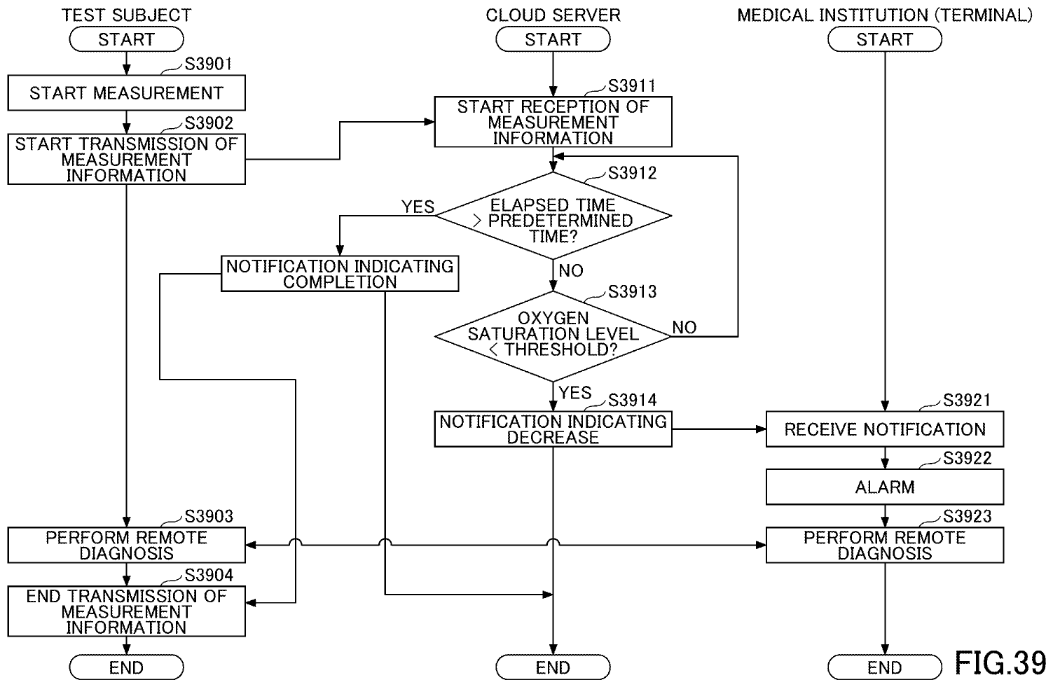

FIG. 39 is a flowchart illustrating processing regarding the alarm function of the diagnostic system according to the present embodiment.

In the test subject, the measurement is started by the sticker-type pulse wave sensor 2500 that is worn (S3901). Then, the communication terminal 2411 of the test subject starts to transmit the measurement information acquired from the sticker-type pulse wave sensor 2500 to the cloud server 2401 (S3902).

When measurement of the test subject is started, as described above, measurement information such as the pulse waveform, oxygen saturation level, pulse rate, and blood pressure value of the test subject is transmitted to the cloud server 2401. Since waveform data such as pulse waveforms have a large amount of data, transmission may be performed in a thinned-out state, for example, by transmitting only waveform data for 10 seconds per minute. The other measurement information is transmitted at a frequency enabling a dynamic change in the state of the test subject, for example, once per second, to be recognized.

Accordingly, the cloud server 2401 (an example of the communication device) starts receiving the measurement information from the communication terminal 2411 of the test subject (S3911). Accordingly, the cloud server 2401 starts measuring the elapsed time.

The cloud server 2401 determines whether or not the elapsed time has exceeded a predetermined time (S3912). When it is determined that the elapsed time does not exceed the predetermined time (NO in S3912), the cloud server 2401 determines whether the oxygen saturation level included in the measurement information is lower than the threshold (S3913). Note that the predetermined time may a period of 24 hours or may be an examination time or the like determined by the physician.

In this embodiment, it is necessary for the physician or the like to set the alarm function to operate. For example, the physician sets an alarm function corresponding to the condition of the test subject from an online diagnosis or the like performed before starting the measurement. For example, the physician sets a threshold of oxygen saturation level for activating the alarm function. Specifically, in a case where the oxygen saturation level of the test subject in the initial state is 98%, the physician sets the terminal to operate an alarm sound when the oxygen saturation level falls below 95%.

Since oxygen saturation levels vary from individual to individual, the alarm function can be operated at a more appropriate timing by setting at the discretion of the physician. Also, for example, when the test subject has an underlying disease in the heart, it is desirable to prioritize the carrying out of treatment such as hospitalization or oxygen suction, as compared with a decrease in oxygen saturation which causes general pneumonia.

If the cloud server 2401 determines that the oxygen saturation level included in the measurement information is greater than or equal to the threshold value (NO in S3913), the cloud server 2401 performs processing again from S3912.

Conversely, if the cloud server 2401 determines that the oxygen saturation level included in the measurement information is lower than the threshold value (YES in S3913), the cloud server 2401 transmits a notification indicating the decrease to the terminal 2402 of the medical institution (S3914). The notification indicating the decrease according to the present embodiment is information indicating that the oxygen saturation of the test subject has decreased (an example of information regarding the oxygen saturation of the test subject).

The terminal 2402 (an example of the communication device) of the medical institution operates the alarm function (S3922) upon receiving the notification indicating the decrease (S3921). The terminal 2402 of the medical institution displays the received notification indicating the decrease on a display device (not illustrated) of the terminal as an alarm function and outputs a warning sound. Thus, the medical personnel can recognize that the oxygen saturation degree of the test subject has decreased.

A physician belonging to the medical institution performs remote diagnosis for the test subject from the terminal 2402 of the medical institution via the communication terminal 2411 of the test subject (S3923 and S3903). As a result, the communication terminal 2411 of the test subject ends the transmission of the measurement information of the test subject from the sticker-type pulse wave sensor 2500 in order to perform the necessary treatment on the test subject (S3904).

Conversely, if it is determined in S3912 that the elapsed time has exceeded the predetermined time (for example, 24 hours) (YES in 3912), the cloud server 2401 notifies the communication terminal 2411 of the test subject by way of the termination notification (S3915).

In accordance with the notification, the communication terminal 2411 of the test subject ends the transmission of the measurement information of the test subject from the sticker-type pulse wave sensor 2500 (S3904).

In the present embodiment, the medical personnel including the physician can watch over the test subject 24 hours a day by, for example, keeping the terminal on hand while on duty, for example. In addition, since the terminal has an alarm function, the condition of the test subject can be confirmed even when the medical personnel is taking a nap or working at home. When an alarm indicating that the oxygen saturation level of the test subject decreased is transmitted, the condition of the test subject can be confirmed directly from the test subject by establishing a connection from terminal 2411 with the communication terminal of the test subject.

The cloud server 2401 according to the present embodiment can recognize that the measurement by the sticker-type pulse wave sensor 2500 is completed. Therefore, the cloud server 2401 notifies the production facility of the sensor ID of the sticker-type pulse wave sensor 2500 whose measurement is completed. Thus, the production facility can assign the sensor ID indicated by the notification to the sticker-type pulse wave sensor 2500 that is to be produced. That is, in the present embodiment, since the sensor ID can be recycled, even if the number of digits of the sensor ID is limited, a sensor ID to be assigned to the sticker-type pulse wave sensor 2500 is unlikely to be used up.

### Thirteenth Embodiment

In the above-described embodiment, the case of the diagnostic system is mainly described. However, detection of the condition of the test subject is useful for applications other than diagnosis. Therefore, a case of a project support system (an example of a meeting support system) is described in the thirteenth embodiment.

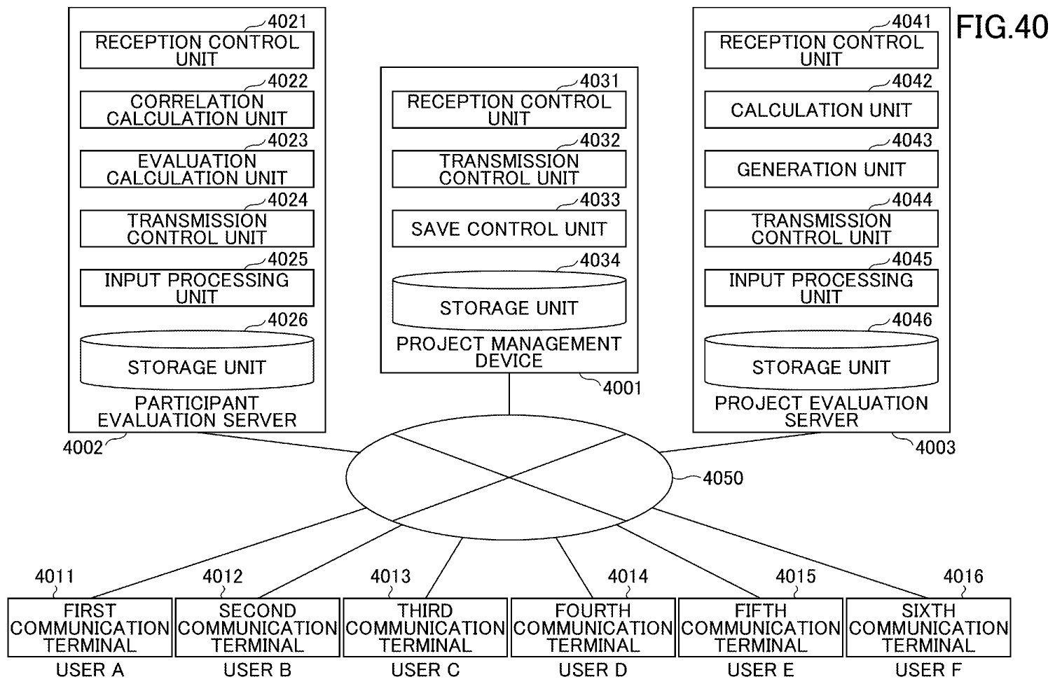

FIG. 40 is a diagram illustrating a configuration example of a project support system according to this embodiment. As illustrated in FIG. 40, the project support system (hereinafter also referred to as a meeting support system for supporting meetings of the project) includes a project management device 4001, a participant evaluation server 4002, and a project evaluation server 4003.

Further, each of users A to F (test subjects) participating in the conference is provided with a different one of the communication terminals among the first communication terminal 4011 to a sixth communication terminal 4016.

The project management device 4001, the participant evaluation server 4002, the project evaluation server 4003, and the first communication terminal 4011 to the sixth communication terminal 4016 are connected by a public network 4050.

The sticker-type pulse wave sensors 2500 and 2900 are worn by the users A to F according to the present embodiment at the above-mentioned four locations. The users A to F serve as participants of the meeting. In this embodiment, the people participating in the meeting are the users A to F. In other words, all participants in the meeting are wearing the sticker-type pulse wave sensors 2500 and 2900. That is, in this embodiment, the participant performs measurements at four locations.

As a specific example, three sticker-type pulse wave sensors 2900 measure three regions of the temporal lobe, DLPFC, and DMPFC of a meeting participant (refer to, for example, FIGS. 29 to 31). Further, the sticker-type pulse wave sensor 2500 measures the position of the upper arm of the meeting participant.

The first communication terminal 4011 to the sixth communication terminal 4016 can transmit the measurement information detected by the sticker-type pulse wave sensors 2500 and 2900 to the participant evaluation server 4002 or the like.

The participant evaluation server 4002 includes a reception control unit 4021, a correlation calculation unit 4022, an evaluation value calculation unit 4023, a transmission control unit 4024, an input processing unit 4025, and a storage unit 4026, and performs evaluation for each participant based on measurement information measured for each participant (for example, the users A to F) who participated in the meeting of the project.

The reception control unit 4021 receives information from an external communication device. For example, in a case where each participant participating in the meeting of this project wears the sticker-type pulse wave sensors 2500 and 2900, the reception control unit 4021 (an example of the acquisition unit) receives the measurement information (information regarding pulse waves) of the participant, which is acquired from the sticker-type pulse wave sensors 2500 and 2900 provided for each participant.

The correlation calculation unit 4022 calculates a correlation coefficient illustrating a correlative relationship in blood pressure change between meetings for each two-participant combination among a plurality of participants participating in the meetings.

An evaluation value calculation unit 4023 calculates and outputs an evaluation value illustrating the evaluation of the participant in the meetings for each participant based on the correlation coefficient calculated for each two-participant combination.

The transmission control unit 4024 transmits information to an external communication device. The input processing unit 4025 receives inputs of information through an input interface.

The storage unit 4026 is a non-volatile recording medium capable of reading and writing. The storage unit 4026 may include, for example, a hard disk drive (HDD) or a solid state drive (SSD).

The project evaluation server 4003 includes a reception control unit 4041, a calculation unit 4042, a generation unit 4043, a transmission control unit 4044, an input processing unit 4045, and a storage unit 4046, and evaluates the project based on the evaluation of each participant who participated in the meetings of the project. Specifically, the project evaluation server 4003 evaluates the project based on of the commitment levels of all participants.

The reception control unit 4041 receives information from an external communication device.

The calculation unit 4042 calculates an evaluation value (an example of the evaluation information) indicating the evaluation of the project for each meeting from the evaluation value of the participant calculated based on of the change in the blood pressure of the participant (an example of fluctuation of pulse wave) acquired during the meeting.

The generation unit 4043 generates and outputs advice for improvements illustrating project evaluation information in which the present project is evaluated, by comparing the present meeting with a past meeting or comparing the cumulative value of the evaluation value calculated for each meeting with the cumulative value of the evaluation value calculated for each meeting in the past project.

The transmission control unit 4044 transmits information to an external communication device. The input processing unit 4045 receives inputs of information through an input interface.

The storage unit 4046 is a non-volatile recording medium capable of reading and writing. The storage unit 4046 may include, for example, a hard disk drive (HDD) or a solid state drive (SSD).

The project management device 4001 includes a reception control unit 4031, a transmission control unit 4032, a save control unit 4033, and a storage unit 4034, and manages the evaluation of each participant and the evaluation of the project.

The reception control unit 4031 receives information from an external communication device. The transmission control unit 4032 transmits information to an external communication device. A save control unit 4033 saves information related to the project in the storage unit 4034.

The storage unit 4034 is non-volatile recording medium capable of reading and writing. The storage unit 4034 may include, for example, a hard disk drive (HDD) or a solid state drive (SSD).

In the meeting support system according to the present embodiment, an appropriate meeting participant by evaluation of each participant and evaluation of the project. In addition, the meeting support system can improve the probability of project success by setting appropriate meeting participants.

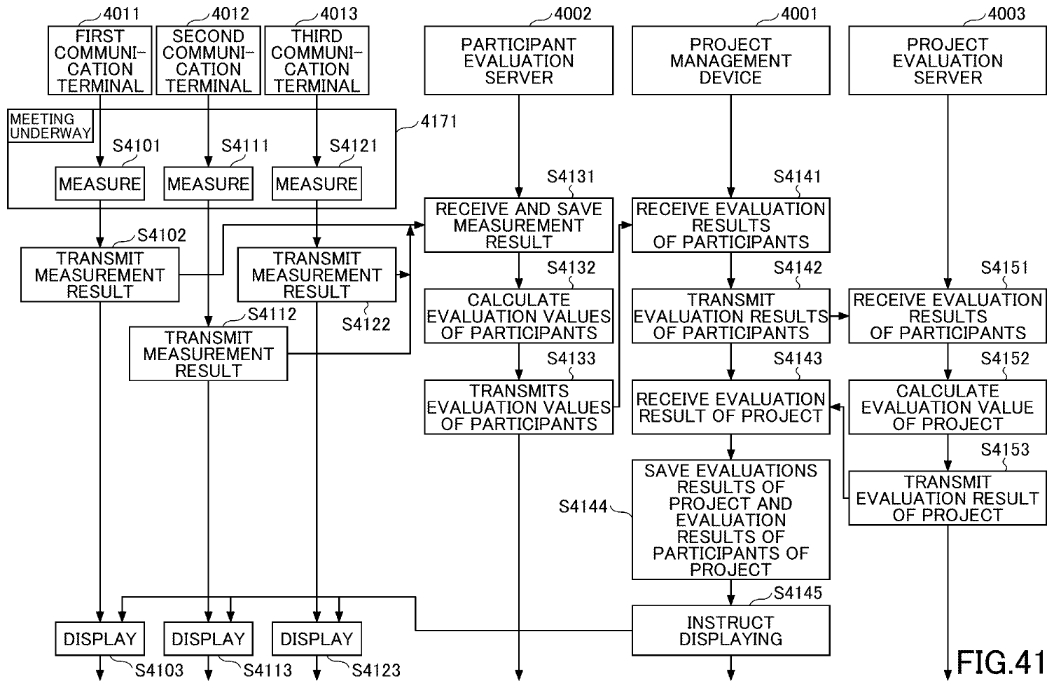

FIG. 41 is a sequence diagram illustrating processing performed in the project support system (also referred to as the meeting support system) according to the present embodiment.

Each of the first communication terminal 4011, the second communication terminal 4012, and the third communication terminal 4013 acquires measurement information detected by the sticker-type pulse wave sensors 2500 and 2900 worn by each meeting participant (for example, users A to C) (S4101, 54111 and S4121). The measurement information is acquired during the meeting 4171. The acquisition of the measurement information is not limited to the first communication terminal 4011, the second communication terminal 4012, and the third communication terminal 4013, but is performed by all the communication terminals of the participants participating in the meeting.

Then, each of the first communication terminal 4011, the second communication terminal 4012, and the third communication terminal 4013 transmits measurement information indicating the measurement result to the participant evaluation server 4002 upon completion of the meeting (S4102, 54112, and S4122).

The reception control unit 4021 of the participant evaluation server 4002 receives measurement information indicating the measurement result of the communication terminal for each user (for example, the first communication terminal 4011, the second communication terminal 4012, and the third communication terminal 4013) (S4131), and the reception control unit 4021 saves the received measurement information in the storage unit 4026.

Then, the correlation calculation unit 4022 and the evaluation value calculation unit 4023 of the participant evaluation server 4002 calculate evaluation values for each participant participating in the meeting based on the saved measurement information (S4132). The information necessary for calculating the evaluation value may be received from an input interface (not illustrated) or the like. The correlation calculation unit 4022 and the evaluation value calculation unit 4023 may store the calculated evaluation values for each participant in the storage unit 4026. A specific method of calculating the evaluation value is described further below.

Thereafter, the transmission control unit 4024 of the participant evaluation server 4002 transmits the evaluation value for each participant to the project management device 4001 (S4133).

Then, the reception control unit 4031 of the project management device 4001 receives the evaluation value for each participant (S4141). The reception control unit 4031 stores the received evaluation value for each participant in the storage unit 4034.

Thereafter, the transmission control unit 4032 of the project management device 4001 transmits the evaluation value for each participant to the project evaluation server 4003 (S4142).

The reception control unit 4041 of the project evaluation server 4003 receives evaluation values for each participant (S4151).

The calculation unit 4042 of a project evaluation server 4003 calculates an evaluation value of the project based on the evaluation value of each participant (S4152). The specific calculation method of the evaluation value of the project is described further below. The evaluation value of the project calculated by the calculation unit 4042 may be stored in the storage unit 4046.

Then, the transmission control unit 4044 of the project evaluation server 4003 transmits the evaluation value of the project to the project management device 4001 (S4153).

The reception control unit 4031 of the project management device 4001 receives the evaluation value of the project (S4143).

The save control unit 4033 of the project management device 4001 saves both the evaluation value of the project and the evaluation value for each participant in the storage unit 4034 in association with the project name (S4144).

Further, the transmission control unit 4032 of the project management device 4001 instructs the communication terminals 4011 to 4013 of the participants to display the evaluation values (S4045).

As a result, each of the first communication terminal 4011, the second communication terminal 4012, and the third communication terminal 4013 displays the evaluation value of the project and the evaluation value for each participant (S4103, 54113, and S4123).

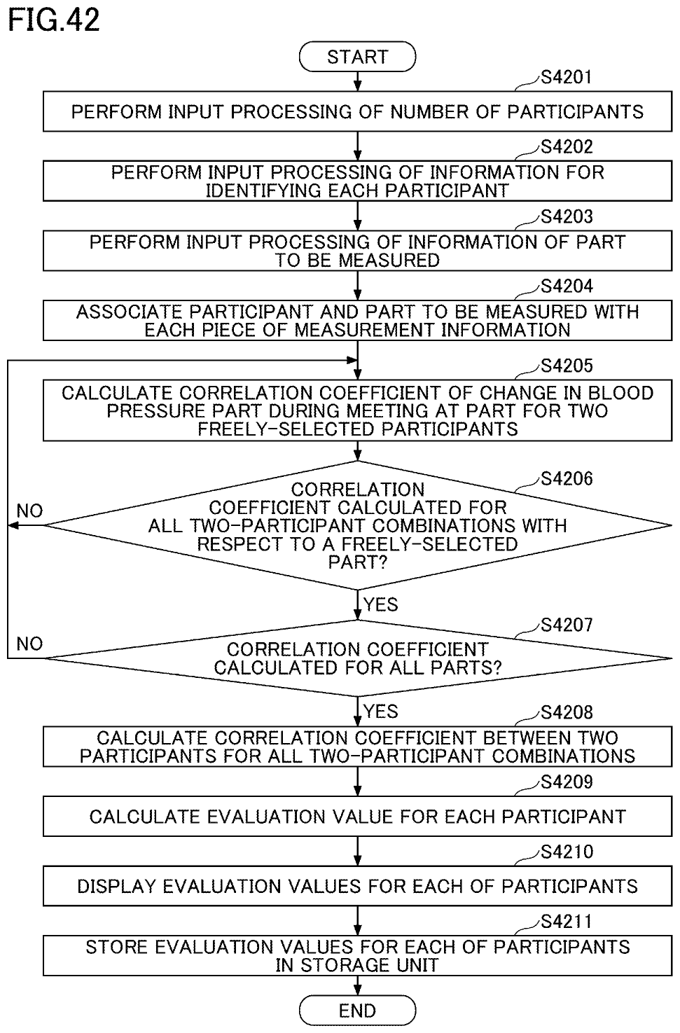

Next, a method of calculating evaluation values for each participant in the participant evaluation server 4002 indicated in S4132 is described. FIG. 42 is a flowchart illustrating a method of calculating an evaluation value for each participant in the participant evaluation server 4002. In this flowchart, measurement information for each participant has already been received (as indicated in S4131 in FIG. 41).

First, the input processing unit 4025 inputs the number of participants participating in the project (S4201).

Next, the input processing unit 4025 performs input processing of information (for example, the name of the participant) for identifying each participant participating in the project (S4202).

Further, the input processing unit 4025 performs input processing on information (for example, the name of the part) of the part to be measured for the participant participating in the project (S4203).

Then, the input processing unit 4025 performs processing for associating a participant and the part to be measured with each piece of measurement information (S4204).

The correlation calculation unit 4022 calculates a correlation coefficient (an example of correlation information) of a change in blood pressure (an example of a fluctuation in the pulse wave) at a freely-selected part during a meeting for each combination of two participants freely selected from among a plurality of participants participating in the meeting (S4205).

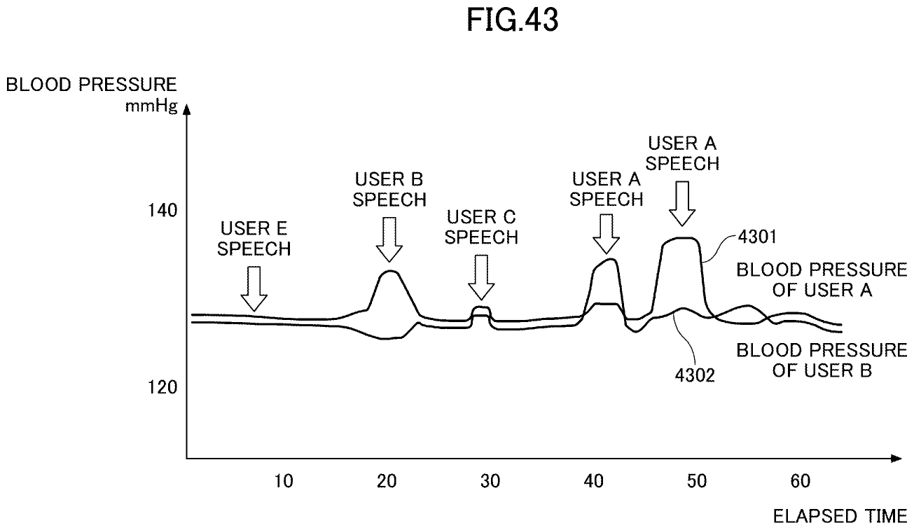

FIG. 43 is a diagram illustrating changes in blood pressure at a predetermined measurement part during a meeting of two participants (user A and user B) in time-series. A line 4301 in FIG. 43 represents a change in the blood pressure of the user A, whereas a line 4302 represents a change in the blood pressure of the user B.

In the example illustrated in FIG. 43, the timings during which a participants is speaking are plotted as indicated by arrows in a meeting that is one hour long. This way it can be recognized that the blood pressure fluctuates at the timing during which the participant speaks. The fluctuation of blood pressure is influenced based on the feelings of the participant with respect to the speech of other participants. In other words, the variation in blood pressure increases or decreases depending on the degree of influence with respect to the speech of other participants. If the variations in blood pressure of two participants are similar, it can be determined that the two participants have similar reactions to the speech of another participant. In other words, the correlation calculation unit 4022 calculates a coefficient (hereinafter referred to as correlation coefficient) representing a correlative relationship between the two participants based on whether or not the variation of the blood pressure value is similar.

That is, in the present embodiment, when there is a correlative relationship between variations in blood pressure caused by the speech of two participants, it is assumed that they have similar feelings about the meeting, and the correlation of feelings about the meeting is estimated. In recent years, there has been a tendency to use a correlative relationship of brain activity as an evaluation function to visualize the minds of test subjects (Ryuta Kawashima, “Making Breakthroughs in ‘Empathic Brains’ for Measuring Communication Quality”, Nikkei Electronics, Inc., Nikkei B P, Jan. 21, 2013, pp. 35 to 37). Cerebral blood flow and blood pressure appear to have a highly correlative relationship in the autonomic nervous system. Therefore, in the present embodiment, a correlative relationship in feelings regarding a meeting between two participants is derived from a correlative relationship of variations in blood pressure.

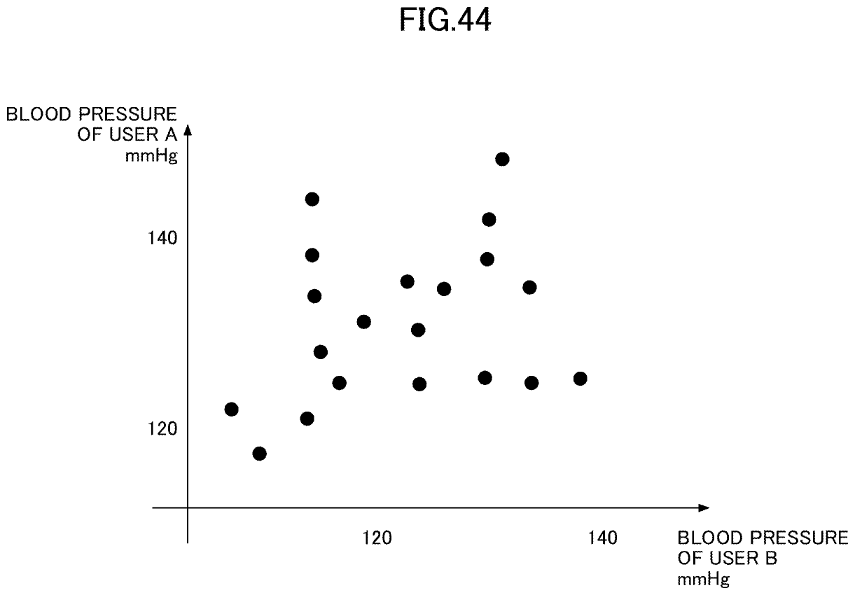

FIG. 44 is a diagram for describing a two-participant correlation coefficient calculation method performed by the correlation calculation unit 4022. In the example illustrated in FIG. 44, the blood pressure of the user A and the blood pressure of the user B are plotted every 30 seconds during a meeting. FIG. 23 plots the blood pressure of the user A on the vertical axis and the blood pressure of the user B on the horizontal axis. For example, in a 60 minute meeting, the combination of the blood pressure of user A and user B is plotted at 120 points.

When the blood pressure combinations are plotted as proportional in FIG. 44, the correlative relationship is high. Proportional means that when the blood pressure of the user A rises, the blood pressure of the user B rises, and also means that when the blood pressure of the user A falls, the blood pressure of the user B falls. The correlation calculation unit 4022 according to the present embodiment calculates a correlation coefficient between the two participants based on the plot. The correlation coefficient may be calculated using any technique, for example, by using Pearson's product-moment correlation coefficient calculation technique.

Referring back to FIG. 42, the correlation calculation unit 4022 determines whether or not all the two-participant combination correlation coefficients have been calculated for a freely-selected part (S4206). If the correlation coefficients have not been calculated for all the two-participant combinations (NO in S4206), the processing returns to S4205, and the correlation calculation unit 4022 calculates the correlation coefficients for the two-participant combinations that have not been calculated.

Thereby, a correlation coefficient is calculated the for two-participant combinations for a freely-selected part. Table 9 illustrates the correlative relationship for each two-participant combination for a freely-selected part (e.g., left temple).

TABLE 9

| | | | | | | Average |
|---|---|---|---|---|---|---|
| | A | B | C | D | E | Value |
| A | | 0.5 | 0.2 | 0.5 | 0.1 | 0.32 |
| B | 0.5 | | 0.1 | 0.3 | 0.5 | 0.35 |
| C | 0.2 | 0.1 | | 0.5 | 0.4 | 0.30 |
| D | 0.5 | 0.3 | 0.5 | | 0.6 | 0.47 |
| E | 0.1 | 0.2 | 0.5 | 0.6 | | 0.35 |
| All | | | | | | 0.36 |
| Participants | | | | | | |

Conversely, if it is determined that the correlation coefficients of all the combinations of the two participants have been calculated for the freely-selected part (YES in S4206), the correlation calculation unit 4022 then determines whether the correlation coefficients have been calculated for all the parts (S4207). If it is determined that the correlation coefficients have not been calculated for all the sites (NO in S4207), the processing returns to S4205, and the correlation calculation unit 4022 calculates the correlation coefficients between the two participants for the not-yet calculated parts.

Conversely, if it is determined that correlation coefficients have been calculated for all the parts (YES in S4207), the correlation calculation unit 4022 integrates the correlative relationship calculated for each part and calculates the correlative relationship between the two participants for all the two-participant combinations (S4208).

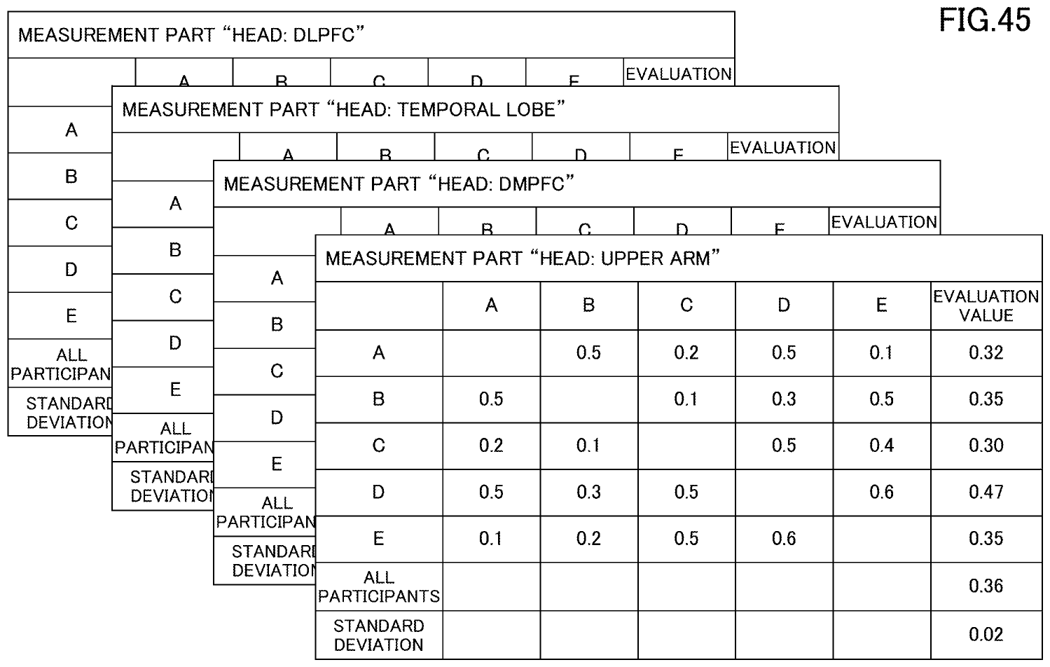

FIG. 45 is a diagram illustrating matrixes of two-participant correlation coefficients for each measurement part calculated by the correlation calculation unit 4022. The matrix-like evaluation result illustrated in FIG. 45 illustrates that the correlative relationship was calculated for each measurement part (e.g., temporal lobe (behind the ear), DLPFC (left temple), DMPFC (between the eyebrows), and upper arm of the meeting participants) to which the sticker-type pulse wave sensors 2500 and 2900 were affixed. The evaluation index of the head differs according to its function. Therefore, the correlation coefficient between the two participants may be calculated by taking into account the evaluation index. For example, when the correlation calculation unit 4022 calculates a participant's evaluation value for a task requiring social cognition and teamwork, the correlation coefficient calculated from the head DMPFC may be multiplied by a predetermined coefficient, and then the level of commitment to the meeting may be evaluated. In this manner, the correlation calculation unit 4022 calculates the correlation coefficient between the two participants by adding up and averaging the correlation coefficients of all the measurement parts between the two participants after performing the correction as described above.

Thereafter, an evaluation value calculation unit 4023 calculates, on a per-participant basis, the average values of the participants as evaluation information illustrating the evaluations of the participants in the meeting based on the correlation coefficient calculated for each two-participant combination (S4209).

The evaluation value calculation unit 4023 (an example of output unit) displays the calculated evaluation value for each participant on a display device (not illustrated) (S4210).

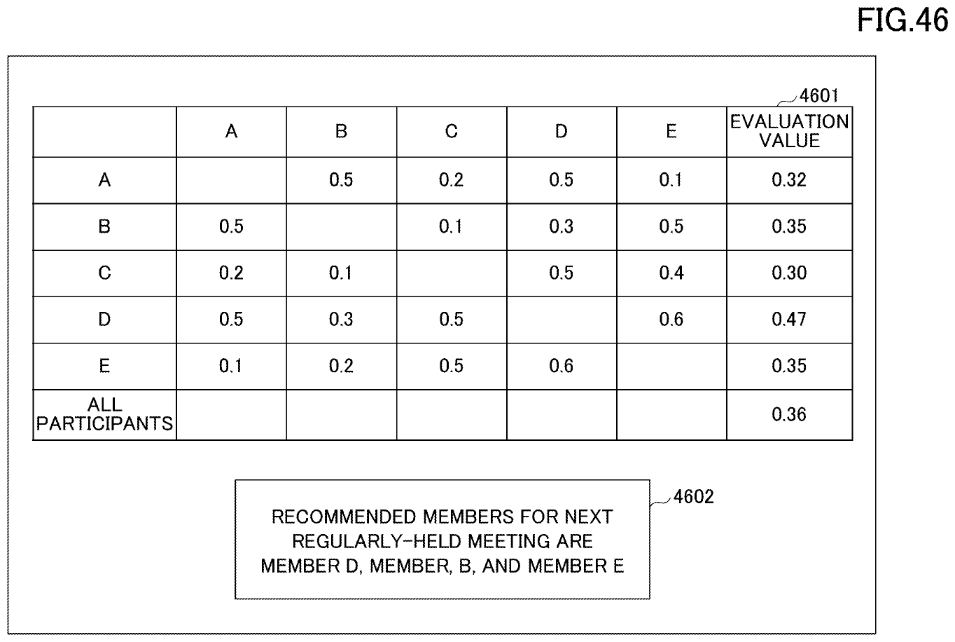

FIG. 46 is a diagram illustrating a screen example illustrating evaluation values for each participant displayed by the evaluation value calculation unit 4023. The screen illustrated in FIG. 46 includes a table 4601 illustrating evaluation values for each participant and a display field 4602 indicating the recommended members. The table 4601 illustrates evaluation values acquired by averaging the correlation coefficients for each of the participants. In the display field 4602, members recommended for the next meeting are displayed in order of the highest evaluation values of the participants. In other words, a participant with a low rating is determined to have a low commitment to the meeting, whereas a participant with a high rating is determined to have a high commitment to the meeting. Therefore, by displaying the recommended members in the order of the highest commitment, the evaluation value calculation unit 4023 can make the next meeting more meaningful and improve the probability of leading the project to success.

Then, the evaluation value calculation unit 4023 stores the calculated evaluation value for each participant in the storage unit 4026 (S4211).

In the present embodiment, according to the above-described processing procedure, an evaluation value of the participant with respect to the meeting can be calculated for each participant participating in the meeting. The host of the next meeting can make the next meeting more beneficial by referring to the evaluation value at the next meeting. That is, at the next meeting, by changing the participants such that those participating have high correlative relationships, those participating can share their feelings and the like about the purpose of the meeting. This makes the meeting more beneficial.

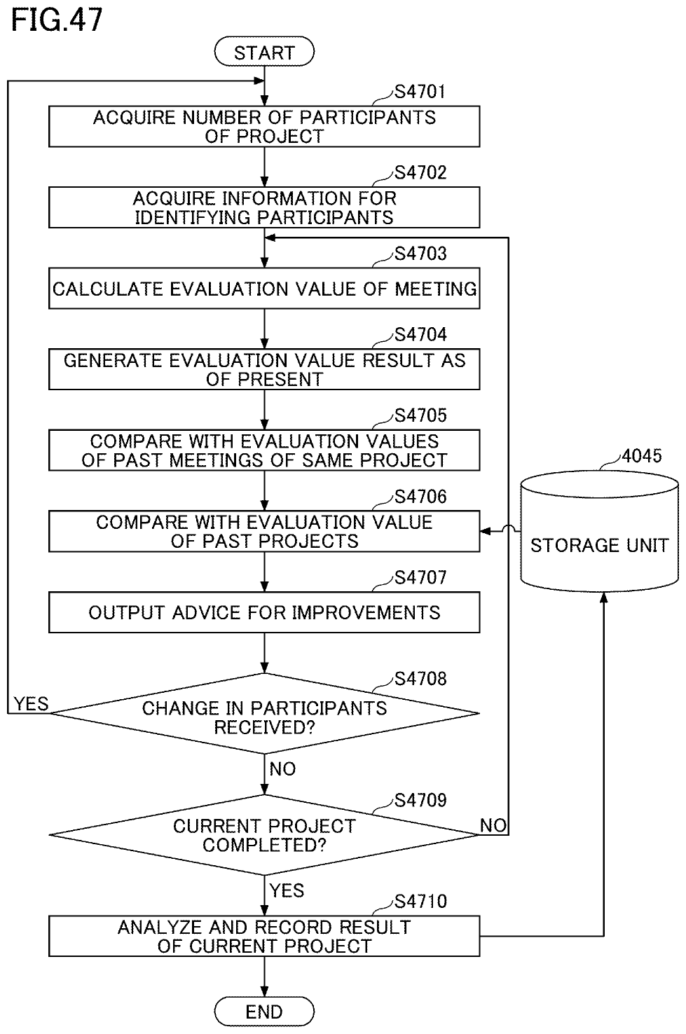

Next, the calculation method of the evaluation value of the project in the project evaluation server 4003 indicated in S4152 is described. FIG. 47 is a flowchart illustrating a calculation technique of calculating a project evaluation value in the project evaluation server 4003. In this flowchart, evaluation values for each participant in the meeting have already been received (as indicated in S4152 in FIG. 41).

First, the input processing unit 4045 acquires the number of participants in the project (S4701). The number of participants may be acquired from the project management device 4001 or may be input via an input interface.

Further, the input processing unit 4045 acquires information (for example, name of participant) for identifying a participant of the project, and does so for each participant in the project (S4702). The information for identifying the participants may be acquired from the project management device 4001, for example, or may be input via the input interface. Thus, when the advice for improvements is output, information for identifying the participant (name of participant) can be displayed.

Thereafter, the calculation unit 4042 calculates the average of the evaluation values of each participant received in S4152 of FIG. 41, and then calculates the evaluation value of the meeting (S4703). The storage unit 4046 of the project evaluation server 4003 stores all the evaluation values calculated with respect to the meeting by the calculation unit 4042. The evaluation value of the meeting is the average of the evaluation values for each of the participants. In other words, since a high participant rating indicates that the participant has a high commitment to the meeting, a high average participant rating indicates that the meeting was beneficial.

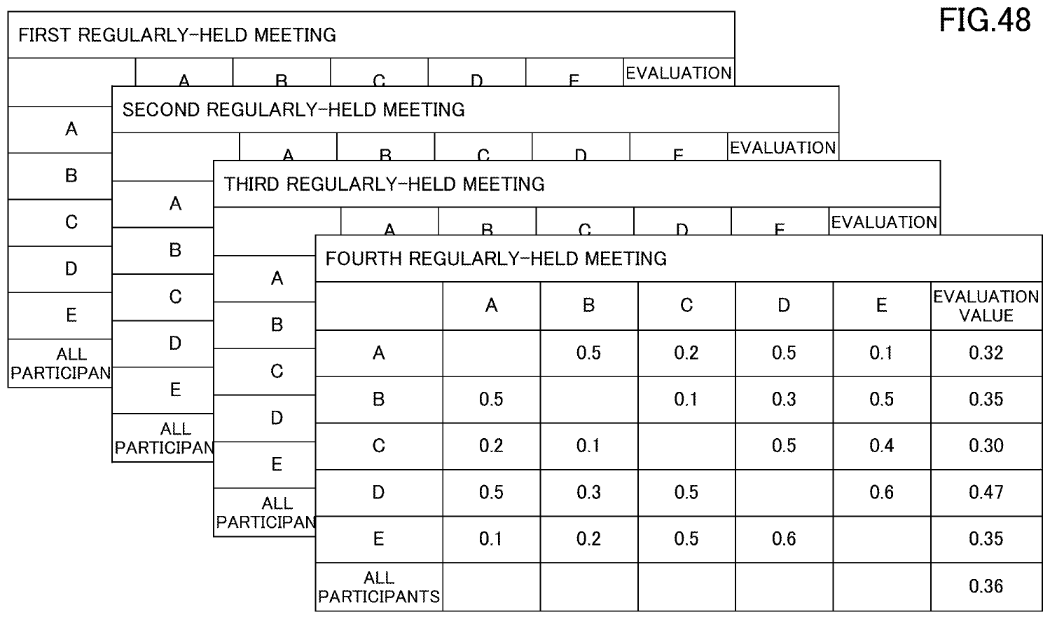

FIG. 48 is a diagram illustrating evaluation values for each meeting (regularly-held meeting) calculated by the calculation unit 4042. By retaining the evaluation values for each meeting in this way, the evaluations of the meetings can be compared and examined.

Referring back to FIG. 47, the calculation unit 4042 generates an evaluation value result at the present time in the current project (S4704). Table 10 illustrates the evaluation results generated by the calculation unit 4042. In the example illustrated in Table 10, the current overall evaluation indicates the evaluation value of the meeting calculated this time.

TABLE 10

| | | | | Narrowed- |
|---|---|---|---|---|
| | Number of | Meeting | Evaluation | down |
| | Participants | Duration | Value | Participants |
| First | 10 | 0.5 | 0.5 | 0 |
| Regularly- | | | | |
| held | | | | |
| Meeting | | | | |
| Second | 9 | 1.5 | 0.6 | 1 |
| Regularly- | | | | |
| held | | | | |
| Meeting | | | | |
| Third | 9 | 0.5 | 0.3 | 1 |
| Regularly- | | | | |
| held | | | | |
| Meeting | | | | |
| Fourth | 7 | 1.5 | 0.7 | 3 |
| Regularly- | | | | |
| held | | | | |
| Meeting | | | | |

| | |
|---|---|
| Overall | 0.7 |
| Evaluation | |
| of Current | |
| Meeting | |

The generation unit 4043 compares the current meeting with the evaluation values of the past meeting of the same project (S4705). The generation unit 4043 can recognize whether or not the evaluation value has increased by comparing with the evaluation values of past meetings as illustrated in FIG. 48.

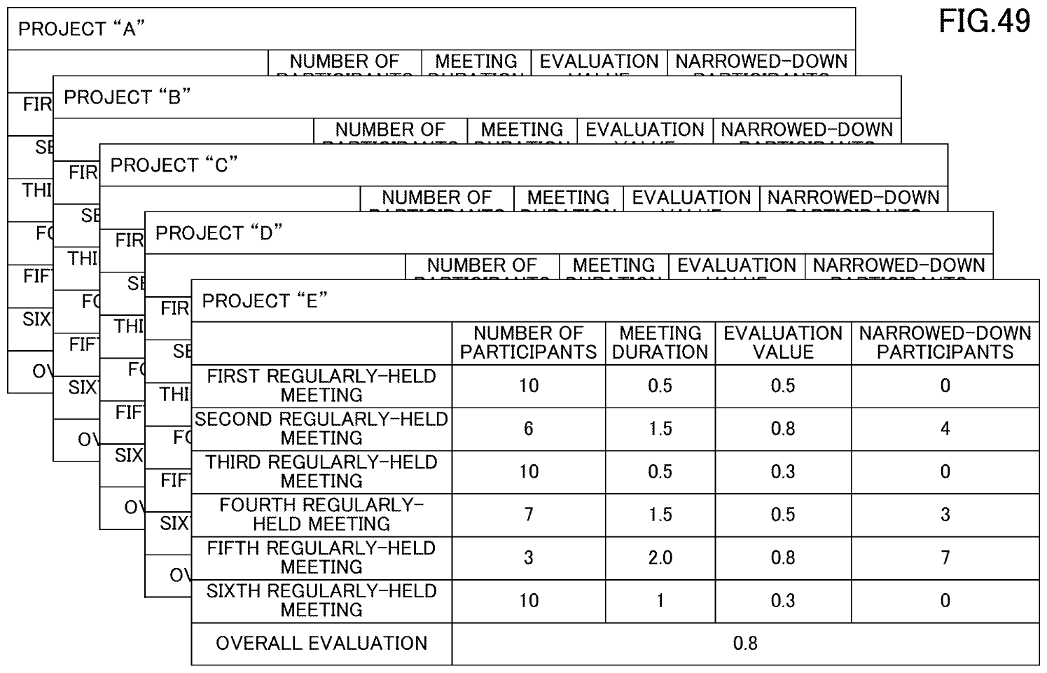

Further, the generation unit 4043 compares the cumulative value of the evaluation value of the meeting of the current project with the cumulative value of the evaluation values of the past projects (S4706). FIG. 49 is a diagram illustrating evaluation values of past projects stored in the project evaluation server 4003 according to the present embodiment in a matrix form. As illustrated in FIG. 49, it is possible to recognize how the evaluation value changed by narrowing down of the participants by the information of each meeting of the past project.

In other words, by referring to the changes in the evaluation values of past project meetings, it is possible to infer how the evaluation values will change due to changes in participants. In addition, the evaluation after the evaluation of the project by the external evaluator or the project leader is input to the past project. That is, information indicating whether the project was successful or not is included. Therefore, by comparing with past projects, it is possible to infer whether the current project is proceeding appropriately or not. Furthermore, the participants may be narrowed down by taking into account the difference in the change in the participants and the change in the evaluation value between successful projects and unsuccessful projects. This can improve the probability of success of the project.

In this way, the generation unit 4043 can recognize what kind of participant narrowing has resulted in an increase in the evaluation value by referring to a past project in a situation close to the present meeting. For example, the generation unit 4043 extracts a past project having an evaluation value close to the current evaluation value. The participant change (participant changes, such as matching the number of successful projects with subsequent participants, or following the successful projects with subsequent evaluation values) of the present project is specified based on the change in the participants of the successful project among the extracted past projects. Then, the generation unit 4043 generates advice for improvements for presenting the specified participant change.

Then, the generation unit 4043 generates advice for improvements based on past meetings and past projects, and outputs the generated advice for improvements (an example of project evaluation information) (S4707).

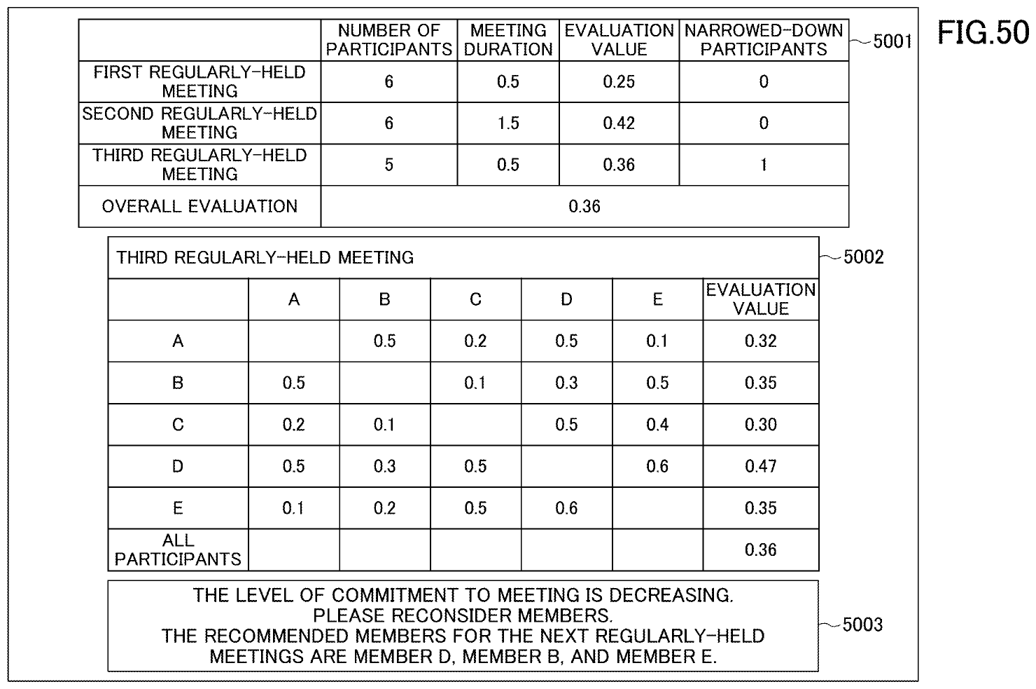

FIG. 50 is a diagram illustrating a screen example of advice for improvements output by the project evaluation server 4003. FIG. 50 illustrates a table 5001 illustrating the evaluation of each meeting of this project, a table 5002 illustrating the evaluation of each participant of this regularly-held meeting, and a message field 5003 for advice for improvements. As illustrated in the message field 5003, when the evaluation value of the current meeting is lower than the evaluation value of the previous meeting of the project, advice for strongly urging the reduction of the number of participants is displayed.

Referring back to FIG. 47, the input processing unit 4045 determines whether or not a change in the participants is received (S4708). If it is determined that the change in the participants is received (YES in S4708), and if the evaluation value for each participant of the next meeting is received, the process starts at S4701.

Conversely, if the input processing unit 4045 determines that the change of the participant is not received (NO in S4708), the input processing unit 4045 determines whether or not the current project is completed (S4709).

If the input processing unit 4045 determines that the current project has not been completed (NO in S4709), and if the evaluation value for each participant of the next meeting is received, the processing starts at S4703.

When the input processing unit 4045 determines that the current project is finished (NO in S4709), the result of the current project is analyzed and recorded in the storage unit 4046 (S4710). At this time, the input processing unit 4045 may input the evaluation of the current project from the outside and record the evaluation of the current project in the storage unit 4046.

In the present embodiment, when evaluating the meeting of the project by performing the above-described processing, the evaluation value of the meeting is derived from the measurement information of all the participants participating in the meeting. This allows for an appropriate evaluation of the meeting. Further, by changing the participants of the next meeting or the like based on the evaluation value of each participant, the meeting can be more beneficial, and the probability of success of the project can be improved.

In the meeting according to the present embodiment, a sub-project for solving problems of the project may be established within the project. In this case, the project management device 4001 may manage the sub-project as information belonging to the project. Further, the project evaluation server 4003 may manage the evaluation value of the meeting performed in the sub-project separately from the evaluation value performed in the project.

In this embodiment, the project management device 4001 manages information related to a new sub-project. In this case, the project management device 4001 may set agenda items relating to the sub-project.

The members of the sub-project may be selected by any technique. For example, the project evaluation server 4003 may select a participant recommended by the advice for improvements illustrated in the message field 5003 as a member of the sub-project. In the present embodiment, the member selection method is not limited to such a method. For example, the project evaluation server 4003 may select the participants recommended in the advice for improvements as candidates for the members, taking into consideration a score based on an emotional change extracted from the participant during the meeting or a score of an action (for example, speech) exhibited during the meeting.

In the present embodiment, by performing the above-described processing, the participants and the like can be changed so as to make the project meeting more beneficial. This increases the probability of success for the project.

In the above-described embodiment, a disposable pulse wave sensor and a disposable sphygmomanometer have been described. However, the above-described embodiment is not limited to a disposable pulse wave sensor and a disposable sphygmomanometer, and is applicable to a biological measurement device for measuring a test subject (medical examination examinee).

### Modified Example

In the system (for example, a diagnostic system or a meeting system) described in the above embodiment, an example in which a test subject or a meeting participant affixes a disposable sticker-type pulse wave sensor has been described. However, the above-described embodiment illustrates an example of a biological measurement device worn by a test subject or a meeting participant, and is not limited to the technique of wearing the biological measurement device described above. In particular, when a meeting participant wears a biological measurement device, a wearable device may be worn instead of affixing the sticker-type pulse wave sensor every time a meeting is held. The shape of the wearable device may be any shape, such as a band that can be worn on the upper arm by a meeting participant or a cap that can be worn on the head by a meeting participant.

The present invention is not limited to these embodiments, and various modifications and substitutions can be made without departing from the spirit of the present invention.

One aspect of the present disclosure is as follows.

<1>
A biological measurement device, including:
   a light emitting unit configured to emit light on a body of a test subject;
   a light detecting unit configured to detect light reflected in the body of the test subject;
   a control unit configured to calculate information regarding a pulse wave of the body of the test subject based on the light detected by the light detecting unit;
   a circuit board that is flexible and has a first surface on which the light emitting unit and the light detecting unit are provided, the circuit board further having wiring connecting the light emitting unit and the control unit together and connecting the light detecting unit and the control unit together;
   a shielding unit that is provided on the first surface, the shielding unit being situated between the light emitting unit and the light detecting unit and configured to protrude beyond the light emitting unit and the light detecting unit in a direction perpendicular to the first surface; and
   an adhesive part for firmly contacting with the body of the test subject.
      <2>
The biological measurement device according to <1>, wherein the shielding unit is configured such that the shielding unit surrounds the light detecting unit on the first surface.
   <3>
The biological measurement device according to <1> or <2>, wherein an end surface of the shielding unit that is to firmly contact with the body of the test subject is formed as the adhesive part.
   <4>
The biological measurement device according to any one of <1> to <3>, further including:
   a member that is provided between the light detection unit and the shielding unit, the member having an end surface that is to firmly contact with the body of the test subject and that is configured to reflect light.
      <5>
A pulse wave sensor, including:
   the biological measurement device according to any one of <1> to <4>.
      <6>
A sphygmomanometer, including:
   the biological measurement device according to any one of <1> to <4>.
      <7>
The sphygmomanometer according to <6>, wherein
   the light detection unit includes a plurality of light detectors such that different positions along the body of the test subject in an artery running direction are detectable, and
   the light emitting unit is individually provided with respect to each of the light detectors, with the corresponding shielding layer interposed therebetween.

<8>
8. The sphygmomanometer according to <6> or <7>, further including:
   an acceleration sensor.
      <9>
The sphygmomanometer according to any one of <6> to <8>, wherein the sphygmomanometer is configured in a size such that the sphygmomanometer is settable in vicinity of a subclavian artery of a person.
   <14>
A diagnostic method, including:
   receiving, by an information processing device, a time-series-based change in blood pressure of a body of a test subject acquired by a biological measurement device wearable by the test subject;
   receiving, by the information processing device, event information indicating events that occurred with respect to the body of the test subject in time series;
      extracting, by the information processing device, the time-based change in the blood pressure based on the event information indicating the event information;
      identifying, by the information processing device, a classification of the acquired change in the blood pressure by performing matching between: the change in blood pressure extracted based on the event; and a change model representing a predetermined change in the blood pressure or each classification representing characteristics of the body of the test subject; and
      outputting, by the information processing device, the identified classification.
(Effect)
   The information processing device stores event information indicating an event identified based on both vitals data (pulse wave, blood pressure, hemoglobin oxygen saturation level, cerebral blood flow in brain region DLPFC, cerebral blood flow in brain region DMPFC, and so on) obtained by a biological measurement device wearable by the test subject, such as a wearable sensor, and information obtained from, for example, a microphone, a temperature sensor, an internal acceleration sensor, or the like in the wearable sensor, and displays the event indicated by the event information together with the vital value, and thus a physician can determine a cause of sudden fluctuations or the like in vital data. For example, the information processing device records the bedtime, medication, early morning toilet visits, and so on, and superimposes the information onto changes in blood pressure, so that the physician can make a diagnosis after excluding changes in blood pressure that are different from the diagnosis target, such as information related to early morning hypertension, thereby improving the diagnosis accuracy. Further, the information processing device performs change-model-based pattern-matching, so that the subtype of hypertension can be calculated. Hence, the diagnosis of the physician can be facilitated.
      <15>
A program for causing a computer to:
   acquire, with respect to a body of each test subject, change in blood pressure of the body of the test subject in time series, the change in the blood pressure being acquired by a wearable biological measurement device; receive, in the time series, conversation event information indicating a timing of conversation for each test subject;
   calculate a score determined based on a change in blood pressure that occurred at a timing of conversation with another test subject; and
   output the calculated score for each test subject.
(Effect)
The information processing device that executed the program analyzes a score based on both vitals data (pulse wave, blood pressure, hemoglobin oxygen saturation level, cerebral blood flow in brain region DLPFC, cerebral blood flow in brain region DMPFC, and so on) obtained by a biological measurement device wearable by the test subject, such as a wearable sensor, and conversation event information obtained from, for example, an internal microphone in the biological measurement device. Specifically, the vital value is measured and the other individual in the conversation of the event is analogically inferred, and then the vital data and the other individual are recorded in association with each other. By doing so, objective data as to what kind of vital value is obtained when a conversation is held with certain somebody. By accumulating such data, insights as to the causes of increases in blood pressure in those who are said to have workplace hypertension can be gained. By knowing the causes, improvements can be made against workplace hypertension.
Simultaneous measurements are made while multiple participates wear the biological measurement devices. The information processing corrects individual differences by quantifying the measured vital values as fluctuation rates or the like with respect to an average value. As a result, a method can be standardized thereby enabling comparisons to be made among multiple test subjects. The voices collected by the microphone can be classified by AI processing, for example, to identify who is speaking with who. By doing so, it can be identified by whom one's vitals are increased the most. By carrying this out with multiple people, for example, people who are susceptible to stress, such as increased blood pressure, and those who inflict stress on others, can be quantified and ranked. By calculating such scores, the information processing device can contribute towards visualization of feelings regarding stress of the test subjects, and this has an effect in reducing the stress level of the group.
   <16>
A meeting support system, including:
   an acquisition unit configured to, in a case where a plurality of participants in a meeting are wearing a wearable biological measurement device, acquire information regarding pulse waves of the participants participating based on the biological measurement device worn by each of the participants;
   a correlation calculation unit configured to calculate, for each two-participant combination among the participants participating in the meeting, correlation information indicating a correlational relationship of fluctuations in pulse waves during the meeting;
   an evaluation calculation unit configured to calculate evaluation information, for each participant, based on the correlation information calculated for each two-participant combination, the evaluation information being information indicating an evaluation of the participant during the meeting; and
an output unit configured to output the evaluation information of each of the participants calculated by the evaluation calculation unit.

### <Effect>

By having all of the participants participating in a meeting wear a biological measurement device, the levels of commitment of all participants with respect to the meeting can be evaluated based on the information regarding the pulse waves of the participants. By referring to this information, the organizer or the like of the meeting can carefully select participants for the next meeting venue, thereby enabling appropriate running of the meeting.
   <17>
A meeting support system, including:
   an acquisition unit configured to, in a case where a plurality of participants in a meeting of a predetermined project are wearing a wearable biological measurement device, acquire information regarding pulse waves of the participants participating based on the biological measurement device worn by each of the participants;
   an evaluation calculation unit configured to calculate evaluation information, for each meeting, based on fluctuations in the pulse waves acquired during the meeting from the participants participating in the meeting, the evaluation information being information indicating an evaluation of the project;
   a generation unit configured to generate, based on a result in which cumulative information of project evaluation information calculated for each meeting by the evaluation calculation unit and cumulative information of the evaluation information calculated for each meeting of a past project are compared, project evaluation information in which the project is evaluated; and
      an output unit configured to output the project evaluation information.

### (Effect)

The biological measurement device can be worn by all participants in the meeting to make all evaluations for the project. This increases the probability of having a successful project.

### CITATION LIST

Patent Document
Patent Document 1: Japanese Translation of PCT Publication No. 2018-518323
Patent Document 2: Japanese Unexamined Patent Application Publication No. 2018-061675

What is claimed is:

1. A biological measurement device, comprising:
a light emitting unit configured to emit light on a body of a test subject;
a light detecting unit configured to detect light reflected in the body of the test subject;
a control unit configured to calculate information regarding a pulse wave of the body of the test subject based on the light detected by the light detecting unit;
a circuit board that is flexible and has a first surface on which the light emitting unit and the light detecting unit are provided, the circuit board further having wiring connecting the light emitting unit and the control unit together and connecting the light detecting unit and the control unit together;
a shielding unit that is provided on the first surface, the shielding unit being situated between the light emitting unit and the light detecting unit and configured to protrude beyond the light emitting unit and the light detecting unit in a direction perpendicular to the first surface; and
an adhesive part for firmly contacting with the body of the test subject.

2. The biological measurement device according to claim 1, wherein the shielding unit is configured such that the shielding unit surrounds the light detecting unit on the first surface.

3. The biological measurement device according to claim 1, wherein an end surface of the shielding unit that is to firmly contact with the body of the test subject is formed as the adhesive part.

4. The biological measurement device according to claim 1, further comprising:
a member that is provided between the light detection unit and the shielding unit, the member having an end surface that is to firmly contact with the body of the test subject and that is configured to reflect light.

5. A pulse wave sensor, comprising:
the biological measurement device according to claim 1.

6. A sphygmomanometer, comprising:
the biological measurement device according to claim 1.

7. The sphygmomanometer according to claim 6, wherein
the light detection unit comprises a plurality of light detectors such that different positions along the body of the test subject in an artery running direction are detectable, and
the light emitting unit is individually provided with respect to each of the light detectors, with the corresponding shielding layer interposed therebetween.

8. The sphygmomanometer according to claim 6, further comprising:
an acceleration sensor.

9. The sphygmomanometer according to claim 6, wherein the sphygmomanometer is configured in a size such that the sphygmomanometer is settable in vicinity of a subclavian artery of a person.

10. A meeting support system, comprising:
an acquisition unit configured to, in a case where a plurality of participants in a meeting are wearing a biological measurement device, acquire information regarding pulse waves of the participants participating based on the biological measurement device worn by each of the participants, the biological measurement device including:
a light emitting unit configured to emit light on a body of a test subject,
a light detecting unit configured to detect light reflected in the body of the test subject,
a control unit configured to calculate information regarding a pulse wave of the body of the test subject based on the light detected by the light detecting unit,
a circuit board that has a first surface on which the light emitting unit and the light detecting unit are provided, and has wiring connecting the light emitting unit and the control unit together and connecting the light detecting unit and the control unit together, and
a shielding unit that is provided on the first surface, the shielding unit being situated between the light emitting unit and the light detecting unit and configured to protrude beyond the light emitting unit and the light detecting unit in a direction perpendicular to the first surface,
a correlation calculation unit configured to calculate, for each two-participant combination among the participants participating in the meeting, correlation information indicating a correlational relationship of fluctuations in pulse waves during the meeting;
an evaluation calculation unit configured to calculate evaluation information, for each participant, based on the correlation information calculated for each two-participant combination, the evaluation information being information indicating an evaluation of the participant during the meeting; and
an output unit configured to output the evaluation information of each of the participants calculated by the evaluation calculation unit.

11. A meeting support system, comprising:
an acquisition unit configured to, in a case where a plurality of participants in a meeting of a predetermined project are wearing a biological measurement device, acquire information regarding pulse waves of the participants participating based on the biological measurement device worn by each of the participants, the biological measurement device including:
a light emitting unit configured to emit light on a body of a test subject,
a light detecting unit configured to detect light reflected in the body of the test subject,
a control unit configured to calculate information regarding a pulse wave of the body of the test subject based on the light detected by the light detecting unit,
a circuit board that has a first surface on which the light emitting unit and the light detecting unit are provided, and has wiring connecting the light emitting unit and the control unit together and connecting the light detecting unit and the control unit together, and
a shielding unit that is provided on the first surface, the shielding unit being situated between the light emitting unit and the light detecting unit and configured to protrude beyond the light emitting unit and the light detecting unit in a direction perpendicular to the first surface,
an evaluation calculation unit configured to calculate evaluation information, for each meeting, based on fluctuations in the pulse waves acquired during the meeting from the participants participating in the meeting, the evaluation information being information indicating an evaluation of the project;
a generation unit configured to generate, based on a result in which cumulative information of project evaluation information calculated for each meeting by the evaluation calculation unit and cumulative information of the evaluation information calculated for each meeting of a past project are compared, project evaluation information in which the project is evaluated; and
an output unit configured to output the project evaluation information.
